SEQUENCE LISTING (125 sequences)

SEQ ID NO: 1 - protein, 11 aa, Artificial Sequence
  Synthetic Construct
QSLYYRDGYTF

SEQ ID NO: 2 - protein, 3 aa, Artificial Sequence
  Synthetic Construct
LSS

SEQ ID NO: 3 - protein, 9 aa, Artificial Sequence
  Synthetic Construct
MQGTHWPYT

SEQ ID NO: 4 - protein, 8 aa, Artificial Sequence
  Synthetic Construct
GFSFSTYA

SEQ ID NO: 5 - protein, 8 aa, Artificial Sequence
  Synthetic Construct
ISGSGGAT

SEQ ID NO: 6 - protein, 9 aa, Artificial Sequence
  Synthetic Construct
KGLWFGEGY

SEQ ID NO: 7 - protein, 3 aa, Artificial Sequence
  Synthetic Construct
QSS

SEQ ID NO: 8 - protein, 3 aa, Artificial Sequence
  Synthetic Construct
RSS

SEQ ID NO: 9 - protein, 9 aa, Artificial Sequence
  Synthetic Construct
LQGTHWPYT

SEQ ID NO: 10 - protein, 9 aa, Artificial Sequence
  Synthetic Construct
KGLWFGEGL

SEQ ID NO: 11 - protein, 9 aa, Artificial Sequence
  Synthetic Construct
KGLWFGEGI

SEQ ID NO: 12 - protein, 9 aa, Artificial Sequence
  Synthetic Construct
FQGTHWPYT

SEQ ID NO: 13 - protein, 8 aa, Artificial Sequence
  Synthetic Construct
GFTFSTYA

SEQ ID NO: 14 - protein, 8 aa, Artificial Sequence
  Synthetic Construct
GFPFSTYA

SEQ ID NO: 15 - protein, 8 aa, Artificial Sequence
  Synthetic Construct
RFSFSTYA

SEQ ID NO: 16 - protein, 11 aa, Artificial Sequence
  Synthetic Construct
QSLYYRSGYTF

SEQ ID NO: 17 - protein, 8 aa, Artificial Sequence
  Synthetic Construct
INGSGGAT

SEQ ID NO: 18 - protein, 8 aa, Artificial Sequence
  Synthetic Construct
ISGSSGAT

SEQ ID NO: 19 - protein, 8 aa, Artificial Sequence
  Synthetic Construct
ISGNGGAT

SEQ ID NO: 20 - protein, 8 aa, Artificial Sequence
  Synthetic Construct
ISGSGQAT

SEQ ID NO: 21 - protein, 8 aa, Artificial Sequence
  Synthetic Construct
ISGSGGTT

SEQ ID NO: 22 - protein, 11 aa, Artificial Sequence
  Synthetic Construct
  VARIANT 7: Xaa = Asp or Ser
QSLYYRXGYTF

SEQ ID NO: 23 - protein, 3 aa, Artificial Sequence
  Synthetic Construct
  VARIANT 1: Xaa = Leu, Gln, or Arg
XSS

SEQ ID NO: 24 - protein, 9 aa, Artificial Sequence
  Synthetic Construct
  VARIANT 1: Xaa = Met, Leu, or Phe
XQGTHWPYT

SEQ ID NO: 25 - protein, 8 aa, Artificial Sequence
  Synthetic Construct
  VARIANT 1: Xaa = Gly or Arg
  VARIANT 3: Xaa = Ser, Thr, or Pro
XFXFSTYA

SEQ ID NO: 26 - protein, 8 aa, Artificial Sequence
  Synthetic Construct
  VARIANT 2: Xaa = Ser or Asn
  VARIANT 4: Xaa = Ser or Asn
  VARIANT 5: Xaa = Gly or Ser
  VARIANT 6: Xaa = Gly or Gln
  VARIANT 7: Xaa = Ala or Thr
IXGXXXXT

SEQ ID NO: 27 - protein, 9 aa, Artificial Sequence
  Synthetic Construct
  VARIANT 9: Xaa = Tyr, Leu, or Ile
KGLWFGEGX

SEQ ID NO: 28 - protein, 5 aa, Artificial Sequence
  Synthetic Construct
SYSMN

SEQ ID NO: 29 - protein, 5 aa, Artificial Sequence
  Synthetic Construct
TYYMH

SEQ ID NO: 30 - protein, 5 aa, Artificial Sequence
  Synthetic Construct
SYGMH

SEQ ID NO: 31 - protein, 5 aa, Artificial Sequence
  Synthetic Construct
SYAMH

SEQ ID NO: 32 - protein, 5 aa, Artificial Sequence
  Synthetic Construct
SYSMN

SEQ ID NO: 33 - protein, 5 aa, Artificial Sequence
  Synthetic Construct
TYAMS

SEQ ID NO: 34 - protein, 5 aa, Artificial Sequence
  Synthetic Construct
  VARIANT 1: Xaa = Thr or Ser
  VARIANT 3: Xaa = Tyr, Gly, Ala, or Ser
  VARIANT 4: Xaa = Met or Ile
  VARIANT 5: Xaa = His, Asn, or Ser
XYXXX

SEQ ID NO: 35 - protein, 17 aa, Artificial Sequence
  Synthetic Construct
SISSSSSYIYYADSVKG

SEQ ID NO: 36 - protein, 17 aa, Artificial Sequence
  Synthetic Construct
IINPSGGSTSYAQKFQG

SEQ ID NO: 37 - protein, 17 aa, Artificial Sequence
  Synthetic Construct
VISYDGSNKYYADSVKG

SEQ ID NO: 38 - protein, 17 aa, Artificial Sequence
  Synthetic Construct
GIIPIFGTANYAQKFQG

SEQ ID NO: 39 - protein, 17 aa, Artificial Sequence
  Synthetic Construct
GISGSGGATHYADSVKG

SEQ ID NO: 40 - protein, 17 aa, Artificial Sequence
  Synthetic Construct
  VARIANT 1: Xaa = Ile, Val, Gly, or Ser
  VARIANT 3: Xaa = Asn, Ser, or Ile
  VARIANT 4: Xaa = Pro, Tyr, Ser, or Gly
  VARIANT 5: Xaa = Ser, Asp, or Ile
  VARIANT 6: Xaa = Gly, Phe, or Ser
  VARIANT 7: Xaa = Gly or Ser
  VARIANT 8: Xaa = Ser, Asn, Thr, Tyr, or Ala
  VARIANT 9: Xaa = Thr, Lys, Ala, or Ile
  VARIANT 10: Xaa = Ser, Tyr, Asn, or His
  VARIANT 13: Xaa = Gln or Asp
  VARIANT 14: Xaa = Lys or Ser
  VARIANT 15: Xaa = Phe or Val
  VARIANT 16: Xaa = Lys or Gln
XIXXXXXXXXYAXXXXG

SEQ ID NO: 41 - protein, 7 aa, Artificial Sequence
  Synthetic Construct
VTDAFDI

SEQ ID NO: 42 - protein, 11 aa, Artificial Sequence
  Synthetic Construct
DLVVPAATLDY

SEQ ID NO: 43 - protein, 9 aa, Artificial Sequence
  Synthetic Construct
ESYGGQFDY

SEQ ID NO: 44 - protein, 16 aa, Artificial Sequence
  Synthetic Construct
DGFGLAVAGPYWYFDL

SEQ ID NO: 45 - protein, 19 aa, Artificial Sequence
  Synthetic Construct
PTRSRDFWSGLGYYYYMDV

SEQ ID NO: 46 - protein, 12 aa, Artificial Sequence
  Synthetic Construct
DFYEAGGWYFDL

SEQ ID NO: 47 - protein, 15 aa, Artificial Sequence
  Synthetic Construct
VGATTSLYYYYGMDV

SEQ ID NO: 48 - protein, 12 aa, Artificial Sequence
  Synthetic Construct
GIIVGPTDAFDI

SEQ ID NO: 49 - protein, 8 aa, Artificial Sequence
  Synthetic Construct
GLWFGEGY

SEQ ID NO: 50 - protein, 14 aa, Artificial Sequence
  Synthetic Construct
RDGSLGVGYYYMDF

SEQ ID NO: 51 - protein, 12 aa, Artificial Sequence
  Synthetic Construct
  VARIANT 1: Xaa = Asp or Gly
  VARIANT 2: Xaa = Phe or Ile
  VARIANT 3: Xaa = Tyr or Ile
  VARIANT 4: Xaa = Glu or Val
  VARIANT 5: Xaa = Ala or Gly
  VARIANT 6: Xaa = Gly or Pro
  VARIANT 7: Xaa = Gly or Thr
  VARIANT 8: Xaa = Trp or Asp
  VARIANT 9: Xaa = Tyr or Ala
  VARIANT 12: Xaa = Leu or Ile
XXXXXXXXXFDX

SEQ ID NO: 52 - protein, 7 aa, Artificial Sequence
  Synthetic Construct
DSSNRAT

SEQ ID NO: 53 - protein, 7 aa, Artificial Sequence
  Synthetic Construct
DASNLDT

SEQ ID NO: 54 - protein, 7 aa, Artificial Sequence
  Synthetic Construct
LGSNRAS

SEQ ID NO: 55 - protein, 7 aa, Artificial Sequence
  Synthetic Construct
AASSLKS

SEQ ID NO: 56 - protein, 7 aa, Artificial Sequence
  Synthetic Construct
GASSRAT

SEQ ID NO: 57 - protein, 7 aa, Artificial Sequence
  Synthetic Construct
KVSNRDS

SEQ ID NO: 58 - protein, 7 aa, Artificial Sequence
  Synthetic Construct
ENDQRPS

SEQ ID NO: 59 - protein, 7 aa, Artificial Sequence
  Synthetic Construct
LSSKRDS

SEQ ID NO: 60 - protein, 7 aa, Artificial Sequence
  Synthetic Construct
  VARIANT 1: Xaa = Leu, Ala, Gly, Lys, or Glu
  VARIANT 2: Xaa = Gly, Ala, Val, Asn, or Ser
  VARIANT 3: Xaa = Ser or Asp
  VARIANT 4: Xaa = Asn, Ser, Gln, or Lys
  VARIANT 5: Xaa = Arg or Leu
  VARIANT 6: Xaa = Ala, Lys, Asp, or Pro
  VARIANT 7: Xaa = Ser or Thr
XXXXXXX

SEQ ID NO: 61 - protein, 9 aa, Artificial Sequence
  Synthetic Construct
LQHNTFPPT

SEQ ID NO: 62 - protein, 9 aa, Artificial Sequence
  Synthetic Construct
QQAKAFPPT

SEQ ID NO: 63 - protein, 9 aa, Artificial Sequence
  Synthetic Construct
MQALQTPYT

SEQ ID NO: 64 - protein, 9 aa, Artificial Sequence
  Synthetic Construct
QQSYSIPYT

SEQ ID NO: 65 - protein, 9 aa, Artificial Sequence
  Synthetic Construct
QQRSNWPLT

SEQ ID NO: 66 - protein, 9 aa, Artificial Sequence
  Synthetic Construct
MQGAHWPPT

SEQ ID NO: 67 - protein, 9 aa, Artificial Sequence
  Synthetic Construct
QQRSNWPPT

SEQ ID NO: 68 - protein, 9 aa, Artificial Sequence
  Synthetic Construct
QSYDFSTVV

SEQ ID NO: 69 - protein, 9 aa, Artificial Sequence
  Synthetic Construct
QQYGSSPGT

SEQ ID NO: 70 - protein, 9 aa, Artificial Sequence
  Synthetic Construct
MQGTHWPYT

SEQ ID NO: 71 - protein, 9 aa, Artificial Sequence
  Synthetic Construct
MQALQTPIT

SEQ ID NO: 72 - protein, 9 aa, Artificial Sequence
  Synthetic Construct
  VARIANT 1: Xaa = Met or Gln
  VARIANT 2: Xaa = Gln or Ser
  VARIANT 3: Xaa = Ala, Ser, Arg, Gly, or Tyr
  VARIANT 4: Xaa = Leu, Tyr, Ser, Ala, Asp, Gly, or Thr
  VARIANT 5: Xaa = Gln, Ser, Asn, His, or Phe
  VARIANT 6: Xaa = Thr, Ile, Trp, or Ser
  VARIANT 7: Xaa = Pro or Thr
  VARIANT 8: Xaa = Tyr, Leu, Pro, Val, Gly, or Ile
  VARIANT 9: Xaa = Thr or Val
XXXXXXXXX

SEQ ID NO: 73 - protein, 11 aa, Artificial Sequence
  Synthetic Construct
RASQSVSSYLA

SEQ ID NO: 74 - protein, 11 aa, Artificial Sequence
  Synthetic Construct
RASQGIDNWLG

SEQ ID NO: 75 - protein, 16 aa, Artificial Sequence
  Synthetic Construct
RSSQSLLHGNGNNYLD

SEQ ID NO: 76 - protein, 11 aa, Artificial Sequence
  Synthetic Construct
RASQNIASYLN

SEQ ID NO: 77 - protein, 12 aa, Artificial Sequence
  Synthetic Construct
RASQSVSNNYLG

SEQ ID NO: 78 - protein, 16 aa, Artificial Sequence
  Synthetic Construct
RSSQSLVYSDGKTYLD

SEQ ID NO: 79 - protein, 12 aa, Artificial Sequence
  Synthetic Construct
RASQSVSSSYLA

SEQ ID NO: 80 - protein, 13 aa, Artificial Sequence
  Synthetic Construct
TRSRGSIASSYVQ

SEQ ID NO: 81 - protein, 16 aa, Artificial Sequence
  Synthetic Construct
RSSQSLYYRDGYTFLD

SEQ ID NO: 82 - protein, 16 aa, Artificial Sequence
  Synthetic Construct
RSSQSLLYSNGYNYLD

SEQ ID NO: 83 - protein, 12 aa, Artificial Sequence
  Synthetic Construct
  VARIANT 8: Xaa = Ser or Asn
  VARIANT 9: Xaa = Ser or Asn
  VARIANT 12: Xaa = Gly or Ala
RASQSVSXXYLX

SEQ ID NO: 84 - protein, 16 aa, Artificial Sequence
  Synthetic Construct
  VARIANT 7: Xaa = Leu, Val, or Tyr
  VARIANT 8: Xaa = His or Tyr
  VARIANT 9: Xaa = Gly, Ser, or Arg
  VARIANT 10: Xaa = Asp or Asn
  VARIANT 12: Xaa = Asn, Lys, or Tyr
  VARIANT 13: Xaa = Asn or Thr
  VARIANT 14: Xaa = Tyr or Phe
RSSQSLXXXXGXXXLD

SEQ ID NO: 85 - protein, 5 aa, Artificial Sequence
  Synthetic Construct
SYAIS

SEQ ID NO: 86 - protein, 163 aa, Artificial Sequence
  Synthetic Construct
DVVMTQSPLSLPVTPGESASISCRSSQSLLHGNGNNYLDWYLQKPGQSPQLLIYLGSNRASGVPDRFSGSGSGTDFTLQISRVEPEDVGVYYCMQALQTPYTFGQGTKLEIKRTVAAPSVFIFPPSDEQLKSGTASVVCLLNNFYPREAKVQWKVDNALQSGN

SEQ ID NO: 87 - protein, 158 aa, Artificial Sequence
  Synthetic Construct
DVVMTQSPSSLSASVGDRVTVTCRASQNIASYLNWYQQKPGKAPKLLIYAASSLKSGVPSRFSGSGSGRDFTLTISSLQPEDFAAYYCQQSYSIPYTFGQGTKVEIKRTVAAPSVFIFPPSDEQLKSGTASVVCLLNNFYPREAKVQWKVDNALQLGN

SEQ ID NO: 88 - protein, 159 aa, Artificial Sequence
  Synthetic Construct
EIVLTQSPGTLSLSPGERATLSCRASQSVSNNYLGWYQQKPGQAPRLLIYGASSRATGVPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGN

SEQ ID NO: 89 - protein, 163 aa, Artificial Sequence
  Synthetic Construct
DVVMTQSPLSLSVTPGEPASISCRSSQSLVYSDGKTYLDWFLQRPGQSPRRLIYKVSNRDSGVSDRFSGSGSGTDFTLKISRVEAEDVGVYYCMQGAHWPPTFGQGTRVEIKRTVAAPSVFIFPPSDEQLKSGTASVVCLLNNFYPREAKVQWKVDNALQSGN

SEQ ID NO: 90 - protein, 159 aa, Artificial Sequence
  Synthetic Construct
EIVLTQSPGTLSLSPGERATLSCRASQSVSSSYLAWYQQKPGQAPRLLIYGASSRATGIPARFSGSGSGTDFTLTISSLEPEDFAVYYCQQRSNWPPTFGPGTKVDIKRTVAAPSVFIFPPSDEQLKSGTASVVCLLNNFYPREAKVQWKVDNALQSGN

SEQ ID NO: 91 - protein, 162 aa, Artificial Sequence
  Synthetic Construct
NFMLTQPHSVSESPGKTVTVSCTRSRGSIASSYVQWYQQRPGRSPTNVIYENDQRPSGVPTRFSGSVDRSSNSASLTISGLETEDEADYYCQSYDFSTVVFGGGTKLTVLSQPKAAPSVTLFPPSSEELQANKATLVCLISDFYPGAVTVAWKADSSPVKAG

SEQ ID NO: 92 - protein, 159 aa, Artificial Sequence
  Synthetic Construct
EIVLTQSPGTLSLSPGERATLSCRASQSVSSSYLAWYQQKPGQAPRLLIYGASSRATGIPDRFSVSGSGTDFTLTISRLEPEDFAVYYCQQYGSSPGTFGQGTKVEIKRTVAAPSVFIFPPSDEQLKSGTASVVCLLNNFYPREAKVQWKVDNALQSGN

SEQ ID NO: 93 - protein, 163 aa, Artificial Sequence
  Synthetic Construct
DVVMTQSPLSLPVTLGQPASISCRSSQSLYYRDGYTFLDWYVQKPGQSPQLLIYLSSKRDSGVPDRISGSGSGTDFTLRISRVEAEDVGVYYCMQGTHWPYTFGQGTKLEIKRTVAAPSVFIFPPSDEQLKSGTASVVCLLNNFYPREAKVQWKVDNALQSGN

SEQ ID NO: 94 - protein, 163 aa, Artificial Sequence
  Synthetic Construct
DVVMTQSPLSLAVTPGEPASISCRSSQSLLYSNGYNYLDWYLQKPGQSPQLLIYLGSNRASGVPDRFSGSGSGTDFTLKISRVEAEDVGVYYCMQALQTPITFGPGTKVDIKRTVAAPSVFIFPPSDEQLKSGTASVVCLLNNFYPREAKVQWKVDNALQSGN

SEQ ID NO: 95 - protein, 158 aa, Artificial Sequence
  Synthetic Construct
EVQLVQSGAEVKKPGASVKVSCRASGFSFTTYYMHWVRQAPGQGLEWMGIINPSGGSTSYAQKFQGRVTMTRDTSTSTVYMELSSLRSEDTAVYYCARDLVVPAATLDYWGQGTLVTVSSASTKGPSVFPLAPSSKSTSGGTAALGCLVKDYFPEPVT

SEQ ID NO: 96 - protein, 156 aa, Artificial Sequence
  Synthetic Construct
EVQLVQTGGGAVQPGRSLRLSCAATGFTFSSYGMHWVRQAPGKGLEWVAVISYDGSNKYYADSVKGRFTISRDNAKNTLYLQMNSLRAEDTAVYYCARESYGGQFDYWGPGTLVTVSSASTKGPSVFPLAPSSKSTSGGTAALGCLVKDYFPEPVT

SEQ ID NO: 97 - protein, 163 aa, Artificial Sequence
  Synthetic Construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDGFGLAVAGPYWYFDLWGRGTLVTVSSASTKGPSVFPLAPSSKSTSGGTAALGCLVKDYFPEPVT

SEQ ID NO: 98 - protein, 166 aa, Artificial Sequence
  Synthetic Construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAGPTRSRDFWSGLGYYYYMDVWGKGTTVTVSSASTKGPSVFPLAPSSKSTSGGTAALGCLVKDYFPEPVT

SEQ ID NO: 99 - protein, 159 aa, Artificial Sequence
  Synthetic Construct
EVQLVQSGGGVVQPGRSLRLSCAASGFTFSSYGMHWVRQAPGKGLEWVAVISYDGSNKHYADSVKGRFTISRDNSKNTLYLQMNSLRAEDTAVYYCARDFYEAGGWYFDLWGRGTLVTVSSASTKGPSVFPLAPSSKSTSGGTAALGCLVKDYFPEPVT

SEQ ID NO: 100 - protein, 161 aa, Artificial Sequence
  Synthetic Construct
EVQLVQSGAEVKKPGSSVKVSCKASGGTFSSYAISWVRQAPGQGLEWMGGIIPIFGTAYAQKFQGRVTITADESTSTAYMELSSLRSEDTAVYYCARVGATTSLYYYYGMDVWGQGTTVTVSSASTKGPSVFPLAPSSKSTSGGTAALGCLVKDYFPEPVT

SEQ ID NO: 101 - protein, 159 aa, Artificial Sequence
  Synthetic Construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARGIIVGPTDAFDIWGQGTTVTVSSASTKGPSVFPLAPSSKSTSGGTAALGCLVKDYFPEPVT

SEQ ID NO: 102 - protein, 155 aa, Artificial Sequence
  Synthetic Construct
QVQLVQSGGGLVQPGGSLRLSCAASGFSFSTYAMSWVRQAPGKGLEWVSGISGSGGATHYADSVKGRFTISRDNSKNTVNLQMNSLRAEDTAVYYCAKGLWFGEGYWGQGTLVTVSSASTKGPSVFPLAPSSKSTSGGTAALGCLVKDYFPEPVT

SEQ ID NO: 103 - protein, 160 aa, Artificial Sequence
  Synthetic Construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGSLGVGYYYMDFWGKGTTVTVSSASTKGPSVFPLAPSSKSTSGGTAALGCLVKDYFPEPVT

SEQ ID NO: 104 - protein, 158 aa, Artificial Sequence
  Synthetic Construct
EIVMTQSPATLSLSPGERATLSCRASQSVSSYLAWYQQKPGQAPRLLIYDSSNRATGIPARFSGSGSGTDFTLTISSLEPEDFATYYCLQHNTFPPTFGQGTKVEIKRTVAAPSVFIFPPSDEQLKSGTASVVCLLNNFYPREAKVQWKVDNALQSGN

SEQ ID NO: 105 - protein, 158 aa, Artificial Sequence
  Synthetic Construct
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKGTVAAPSVFIFPPSDEQLKSGTASVVCLLNNFYPREAKVQWKVDNALQSGN

SEQ ID NO: 106 - protein, 154 aa, Artificial Sequence
  Synthetic Construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVFPLAPSSKSTSGGTAALGCLVKDYFPEPVT

SEQ ID NO: 107 - protein, 163 aa, Artificial Sequence; identical to SEQ ID NO: 93
  Synthetic Construct

SEQ ID NO: 108 - protein, 163 aa, Artificial Sequence
  Synthetic Construct
DVVMTQSPLSLPVTLGQPASISCRSSQSLYYRDGYTFLDWYVQKPGQSPQLLIYQSSKRDSGVPDRISGSGSGTDFTLRISRVEAEDVGVYYCMQGTHWPYTFGQGTKLEIKRTVAAPSVFIFPPSDEQLKSGTASVVCLLNNFYPREAKVQWKVDNALQSGN

SEQ ID NO: 109 - protein, 163 aa, Artificial Sequence
  Synthetic Construct
DVVMTQSPLSLPVTLGQPASISCRSSQSLYYRDGYTFLDWYVQKPGQSPQLLIYRSSKRDSGVPDRISGSGSGTDFTLRISRVEAEDVGVYYCLQGTHWPYTFGQGTKLEIKRTVAAPSVFIFPPSDEQLKSGTASVVCLLNNFYPREAKVQWKVDNALQSGN

SEQ ID NO: 110 - protein, 163 aa, Artificial Sequence
  Synthetic Construct
DVVMTQSPLSLPVTLGQPASISCRSSQSLYYRDGYTFLDWYVQKPGQSPQLLIYQSSKRDSGVPDRISGSGSGTDFTLRISRVEAEDVGVYYCLQGTHWPYTFGQGTKLEIKRTVAAPSVFIFPPSDEQLKSGTASVVCLLNNFYPREAKVQWKVDNALQSGN

SEQ ID NO: 111 - protein, 163 aa, Artificial Sequence
  Synthetic Construct
DVVMTQSPLSLPVTLGQPASISCRSSQSLYYRDGYTFLDWYVQKPGQSPQLLIYQSSKRDSGVPDRISGSGSGTDFTLRISRVEAEDVGVYYCFQGTHWPYTFGQGTKLEIKRTVAAPSVFIFPPSDEQLKSGTASVVCLLNNFYPREAKVQWKVDNALQSGN

SEQ ID NO: 112 - protein, 163 aa, Artificial Sequence
  Synthetic Construct
DVVMTQSPLSLPVTLGQPASISCRSSQSLYYRSGYTFLDWYVQKPGQSPQLLIYQSSKRDSGVPDRISGSGSGTDFTLRISRVEAEDVGVYYCMQGTHWPYTFGQGTKLEIKRTVAAPSVFIFPPSDEQLKSGTASVVCLLNNFYPREAKVQWKVDNALQSGN

SEQ ID NO: 113 - protein, 163 aa, Artificial Sequence
  Synthetic Construct
DVVMTQSPLSLPVTLGQPASISCRSSQSLYYRSGYTFLDWYVQKPGQSPQLLIYQSSKRDSGVPDRISGSGSGTDFTLRISRVEAEDVGVYYCFQGTHWPYTFGQGTKLEIKRTVAAPSVFIFPPSDEQLKSGTASVVCLLNNFYPREAKVQWKVDNALQSGN

SEQ ID NO: 114 - protein, 155 aa, Artificial Sequence; identical to SEQ ID NO: 102
  Synthetic Construct

SEQ ID NO: 115 - protein, 155 aa, Artificial Sequence
  Synthetic Construct
QVQLVQSGGGLVQPGGSLRLSCAASGFSFSTYAMSWVRQAPGKGLEWVSGISGSGGATHYADSVKGRFTISRDNSKNTVNLQMNSLRAEDTAVYYCAKGLWFGEGLWGQGTLVTVSSASTKGPSVFPLAPSSKSTSGGTAALGCLVKDYFPEPVT

SEQ ID NO: 116 - protein, 155 aa, Artificial Sequence
  Synthetic Construct
QVQLVQSGGGLVQPGGSLRLSCAASGFSFSTYAMSWVRQAPGKGLEWVSGISGSGGATHYADSVKGRFTISRDNSKNTVNLQMNSLRAEDTAVYYCAKGLWFGEGIWGQGTLVTVSSASTKGPSVFPLAPSSKSTSGGTAALGCLVKDYFPEPVT

SEQ ID NO: 117 - protein, 155 aa, Artificial Sequence
  Synthetic Construct
QVQLVQSGGGLVQPGGSLRLSCAASGFTFSTYAMSWVRQAPGKGLEWVSGINGSGGATHYADSVKGRFTISRDNSKNTVNLQMNSLRAEDTAVYYCAKGLWFGEGYWGQGTLVTVSSASTKGPSVFPLAPSSKSTSGGTAALGCLVKDYFPEPVT

SEQ ID NO: 118 - protein, 155 aa, Artificial Sequence
  Synthetic Construct
QVQLVQSGGGLVQPGGSLRLSCAASGFTFSTYAMSWVRQAPGKGLEWVSGINGSGGATHYADSVKGRFTISRDNSKNTVNLQMNSLRAEDTAVYYCAKGLWFGEGLWGQGTLVTVSSASTKGPSVFPLAPSSKSTSGGTAALGCLVKDYFPEPVT

SEQ ID NO: 119 - protein, 155 aa, Artificial Sequence
  Synthetic Construct
QVQLVQSGGGLVQPGGSLRLSCAASGFSFSTYAMSWVRQAPGKGLEWVSGISGSSGATHYADSVKGRFTISRDNSKNTVNLQMNSLRAEDTAVYYCAKGLWFGEGYWGQGTLVTVSSASTKGPSVFPLAPSSKSTSGGTAALGCLVKDYFPEPVT

SEQ ID NO: 120 - protein, 155 aa, Artificial Sequence
  Synthetic Construct
QVQLVQSGGGLVQPGGSLRLSCAASGFSFSTYAMSWVRQAPGKGLEWVSGISGSSGATHYADSVKGRFTISRDNSKNTVNLQMNSLRAEDTAVYYCAKGLWFGEGLWGQGTLVTVSSASTKGPSVFPLAPSSKSTSGGTAALGCLVKDYFPEPVT

SEQ ID NO: 121 - protein, 155 aa, Artificial Sequence
  Synthetic Construct
QVQLVQSGGGLVQPGGSLRLSCAASGFPFSTYAMSWVRQAPGKGLEWVSGISGNGGATHYADSVKGRFTISRDNSKNTVNLQMNSLRAEDTAVYYCAKGLWFGEGYWGQGTLVTVSSASTKGPSVFPLAPSSKSTSGGTAALGCLVKDYFPEPVT

SEQ ID NO: 122 - protein, 155 aa, Artificial Sequence
  Synthetic Construct
QVQLVQSGGGLVQPGGSLRLSCAASRFSFSTYAMSWVRQAPGKGLEWVSGISGSGQATHYADSVKGRFTISRDNSKNTVNLQMNSLRAEDTAVYYCAKGLWFGEGYWGQGTLVTVSSASTKGPSVFPLAPSSKSTSGGTAALGCLVKDYFPEPVT

SEQ ID NO: 123 - protein, 155 aa, Artificial Sequence
  Synthetic Construct
QVQLVQSGGGLVQPGGSLRLSCAASGFSFSTYAMSWVRQAPGKGLEWVSGISGSGGTTHYADSVKGRFTISRDNSKNTVNLQMNSLRAEDTAVYYCAKGLWFGEGYWGQGTLVTVSSASTKGPSVFPLAPSSKSTSGGTAALGCLVKDYFPEPVT

SEQ ID NO: 124 - protein, 155 aa, Artificial Sequence
  Synthetic Construct
QVQLVQSGGGLVQPGGSLRLSCAASGFSFSTYAMSWVRQAPGKGLEWVSGISGSGGTTHYADSVKGRFTISRDNSKNTVNLQMNSLRAEDTAVYYCAKGLWFGEGLWGQGTLVTVSSASTKGPSVFPLAPSSKSTSGGTAALGCLVKDYFPEPVT

SEQ ID NO: 125 - protein, 17 aa, Artificial Sequence
  Synthetic Construct
VISYDGSNKHYADSVKG

(19) United States
(12) Patent Application Publication
(10) Pub. No.: US 2018/0193456 A1
(43) Pub. Date: Jul. 12, 2018

(54) ANTI-VASCULAR ENDOTHELIAL GROWTH FACTOR RECEPTOR 2 (VEGFR2) ANTIBODIES

(71) Applicant: Henlius Biotech Co., Ltd., New Taipei City (TW)

(72) Inventors: Wei-Dong JIANG, San Jose, CA (US); Pei-Hua LIN, Sunnyvale, CA (US); Chi-Ling TSENG, Taipei City (TW)

(21) Appl. No.: 15/740,790

(22) PCT Filed: Jun. 29, 2016
(86) PCT No.: PCT/US2016/040183

§ 371 Date: Dec. 28, 2017

Related U.S. Application Data
(60) Provisional application No. 62/187,204, filed on Jun. 30, 2015.

(51) Int. Cl. A61K 39/395; A61K 39/44; A61P 11/00; A61P 35/00; G01N 33/74; C07K 14/71; C07K 16/28

(52) U.S. Cl. CPC A61K 39/39558; A61K 39/44; A61P 11/00; A61K 2039/505; G01N 33/74; C07K 14/71; C07K 16/2863; A61P 35/00

(57) ABSTRACT

Provided are anti-vascular endothelial growth factor receptor 2 (VEGFR2) antibodies, and antigen binding fragments thereof. Also provided are isolated nucleic acid molecules that encode the anti-VEGFR2 antibodies or antigen binding fragments thereof, related expression vectors, and host cells. Provided are methods of making anti-VEGFR2 antibodies and antigen binding fragments thereof. Also provided are related pharmaceutical compositions comprising anti-VEGFR2 antibodies (or antigen binding fragments thereof) and methods of their use in the treatment of pathological conditions characterized by excessive angiogenesis.

### CROSS-REFERENCE TO RELATED APPLICATIONS

This application claims the priority benefit of U.S. Provisional Application Ser. No. 62/187,204, filed Jun. 30, 2015, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 719902000240SEQLIST.TXT, date recorded: Jun. 28, 2016, size: 75 KB).

### BACKGROUND OF THE INVENTION

Vascular endothelial growth factor receptor (also known as VEGFR2, KDR, FLK1, and CD309) is a cell surface receptor of the VEGF of receptors, a subfamily of three closely related receptor tyrosine kinases, including VEGFR1 (FLT, FLT1), VEGFR2, and VEGFR3 (FLT4, PCL). Binding of VEGFR2 to a ligand (such as VEGE A, C, D, or E) induces receptor dimerization and autophosphorylation of several tyrosine (Y) residues in the C-terminal domain of VEGFR2. This autophosphorylation elicits downstream activation of several signal transduction cascades resulting in endothelial cell proliferation and migration, vascular permeability, and angiogenesis.

Mutations, amplifications or misregulations of VEGFR2 or family members are implicated in epithelial cancers. For example, a relatively high frequency (9%) of mutation and amplification of VEGFR2 has been detected in lung adenocarcinoma. Additionally, studies in NSCLC tumors showed high frequency of VEGFR2 copy number change (32%) in lung adenocarcinoma and squamous cell carcinoma leading to higher VEGFR-2 protein expression. The identification of VEGFR2 as an oncogene has led to the need for the development of anticancer therapeutics directed against VEGFR2. The present invention meets this and other needs.

### BRIEF SUMMARY OF THE INVENTION

Provided herein is an anti-VEGFR2 antibody or antigen binding fragment thereof, wherein the antibody or antigen binding fragment thereof does not block the binding of VEGFR2 to VEGF. Also provided herein is an anti-VEGFR2 antibody or antigen binding fragment thereof, wherein the antibody or antigen binding fragment thereof binds domains 5-7 of VEGFR2. Also provided herein is an anti-VEGFR2 antibody or antigen binding fragment thereof, wherein the antibody or antigen binding fragment thereof does not block the binding of VEGFR2 to VEGF, and wherein the antibody or antigen binding fragment thereof binds domains 5-7 of VEGFR2.

In certain embodiments according to (or as applied to) any of the embodiments above, the anti-VEGFR2 antibody or antigen binding fragment thereof binds to whole HUVEC cells. In certain embodiments according to (or as applied to) any of the embodiments above, the anti-VEGFR2 antibody or antigen binding fragment thereof does not inhibit angiogenesis in vitro. In certain embodiments according to (or as applied to) any of the embodiments above, the anti-VEGFR2 antibody or antigen binding fragment thereof inhibits angiogenesis in vivo. In certain embodiments according to (or as applied to) any of the embodiments above, the anti-VEGFR2 antibody or antigen binding fragment thereof does not inhibit angiogenesis in vitro, and wherein the antibody or antigen binding fragment thereof inhibits angiogenesis in vivo.

Provided herein is an anti-vascular endothelial growth factor receptor 2 (VEGFR2) antibody or antigen binding fragment thereof comprising a light chain variable domain sequence comprising (1) a CDR-L1 comprising the amino acid sequence RASQNIASYLN (SEQ ID NO: 76) or RASQSVS-S/N-S/N-YL-G/A (SEQ ID NO: 83) or TRSRGSIASSYVQ (SEQ ID NO: 80) or RSSQSL-L/V/Y-H/Y-G/S/R-D/N-G-N/K/Y-N/T-Y/F-LD (SEQ ID NO: 84); (2) a CDR-L2 comprising the amino acid sequence L/A/G/K/E-G/A/V/N/S-S/D-N/S/Q/K-R/L-A/K/D/P-S/T (SEQ ID NO: 60); and (3) a CDR-L3 comprising the amino acid sequence M/Q-Q/S-A/S/R/G/Y-L/Y/S/A/D/G/T-Q/S/N/H/F-T/I/W/S-P/T-Y/L/P/V/G/I-T/V (SEQ ID NO: 72); and a heavy chain variable domain sequence comprising (1) a CDR-H1 comprising the amino acid sequence T/S-Y-Y/G/A/S-M/I-H/N/S (SEQ ID NO: 34); (2) a CDR-H2 comprising the amino acid sequence I/V/G/S-I-N/S/I-P/Y/S/G-S/D/I-G/F/S-G/S-S/N/T/Y/A-T/K/A/I-S/Y/N/H-YA-Q/D-K/S-F/V-K/Q-G (SEQ ID NO: 40); and (3) a CDR-H3 comprising the amino acid sequence GLWFGEGY (SEQ ID NO: 49) or ESYGGQFDY (SEQ ID NO: 43) or DLVVPAATLDY (SEQ ID NO:42) or D/G-F/I-Y/I-E/V-A/G-G/P-G/T-W/D-Y/A-FD-L/I (SEQ ID NO: 51) or RDGSLGVGYYYMDF (SEQ ID NO: 50) or VGATTSLYYYYGMDV (SEQ ID NO: 47) or DGFGLAVAGPYWYFDL (SEQ ID NO: 44) or PTRSRDFWSGLGYYYYMDV (SEQ ID NO: 45).

In certain embodiments according to (or as applied to) any of the embodiments above, the light chain variable domain sequence comprises (1) a CDR-L1 comprising an amino acid sequence selected from the group consisting of SEQ ID NOs: 75-82; (2) a CDR-L2 comprising an amino acid sequence selected from the group consisting of SEQ ID NOs: 54-59; and (3) a CDR-L3 comprising an amino acid sequence selected from the group consisting of SEQ ID NOs: 63-71; and a heavy chain variable domain sequence comprising (1) a CDR-H1 comprising an amino acid sequence selected from the group consisting of SEQ ID NOs: 29-33 and 85; (2) a CDR-H2 comprising an amino acid sequence selected from the group consisting of SEQ ID NOs: 35-39 and 125; and (3) a CDR-H3 comprising an amino acid sequence selected from the group consisting of SEQ ID NOs: 42-50.

In certain embodiments according to (or as applied to) any of the embodiments above, the light chain variable domain sequence comprises (1) a CDR-L1 comprising the amino acid sequence RSSQSLLHGNGNNYLD (SEQ ID NO: 75); (2) a CDR-L2 comprising the amino acid sequence LGSNRAS (SEQ ID NO: 54); and (3) a CDR-L3 comprising the amino acid sequence MQALQTPYT (SEQ ID NO: 63); and a heavy chain variable domain sequence comprising (1) a CDR-H1 comprising the amino acid sequence TYYMH (SEQ ID NO: 29); (2) a CDR-H2 comprising the amino acid sequence IINPSGGSTSYAQKFQG (SEQ ID NO: 36); and (3) a CDR-H3 comprising the amino acid sequence DLVVPAATLDY (SEQ ID NO: 42).

In certain embodiments according to (or as applied to) any of the embodiments above, the light chain variable domain sequence comprises (1) a CDR-L1 comprising the amino acid sequence RASQNIASYLN (SEQ ID NO: 76); (2) a CDR-L2 comprising the amino acid sequence AASSLKS (SEQ ID NO: 55); and (3) a CDR-L3 comprising the amino acid sequence QQSYSIPYT (SEQ ID NO: 64); and a heavy chain variable domain sequence comprising (1) a CDR-H1 comprising the amino acid sequence SYGMH (SEQ ID NO: 30); (2) a CDR-H2 comprising the amino acid sequence VISYDGSNKYYADSVKG (SEQ ID NO: 37); and (3) a CDR-H3 comprising the amino acid sequence ESYGGQFDY (SEQ ID NO: 43).

In certain embodiments according to (or as applied to) any of the embodiments above, the light chain variable domain sequence comprises (1) a CDR-L1 comprising the amino acid sequence RASQSVSNNYLG (SEQ ID NO: 77) (2) a CDR-L2 comprising the amino acid sequence GASSRAT (SEQ ID NO: 56); and (3) a CDR-L3 comprising the amino acid sequence QQRSNWPLT (SEQ ID NO: 65); and a heavy chain variable domain sequence comprising (1) a CDR-H1 comprising the amino acid sequence SYAMH (SEQ ID NO: 31); (2) a CDR-H2 comprising the amino acid sequence VISYDGSNKYYADSVKG (SEQ ID NO: 37); and (3) a CDR-H3 comprising the amino acid sequence DGFGLAVAGPYWYFDL (SEQ ID NO: 44).

In certain embodiments according to (or as applied to) any of the embodiments above, the light chain variable domain sequence comprises (1) a CDR-L1 comprising the amino acid sequence RSSQSLVYSDGKTYLD (SEQ ID NO: 78); (2) a CDR-L2 comprising the amino acid sequence KVSNRDS (SEQ ID NO: 57); and (3) a CDR-L3 comprising the amino acid sequence MQGAHWPPT (SEQ ID NO: 66); and a heavy chain variable domain sequence comprising (1) a CDR-H1 comprising the amino acid sequence SYAIS (SEQ ID NO: 85); (2) a CDR-H2 comprising the amino acid sequence GIIPIFGTANYAQKFQG (SEQ ID NO: 38); and (3) a CDR-H3 comprising the amino acid sequence PTRSRDFWSGLGYYYYMDV (SEQ ID NO: 45).

In certain embodiments according to (or as applied to) any of the embodiments above, the light chain variable domain sequence comprises (1) a CDR-L1 comprising the amino acid sequence RASQSVSSSYLA (SEQ ID NO: 79); (2) a CDR-L2 comprising the amino acid sequence set forth in GASSRAT (SEQ ID NO: 56); and (3) a CDR-L3 comprising the amino acid sequence QQRSNWPPT (SEQ ID NO: 67) and a heavy chain variable domain sequence comprising (1) a CDR-H1 comprising the amino acid sequence SYGMH (SEQ ID NO: 30); (2) a CDR-H2 comprising the amino acid sequence set forth in VISYDGSNKHYADSVKG (SEQ ID NO: 125); and (3) a CDR-H3 comprising the amino acid sequence set forth in DFYEAGGWYFDL (SEQ ID NO: 46).

In certain embodiments according to (or as applied to) any of the embodiments above, the light chain variable domain sequence comprises (1) a CDR-L1 comprising the amino acid sequence TRSRGSIASSYVQ (SEQ ID NO: 80); (2) a CDR-L2 comprising the amino acid sequence ENDQRPS (SEQ ID NO: 58); and (3) a CDR-L3 comprising the amino acid sequence QSYDFSTVV (SEQ ID NO: 68); and a heavy chain variable domain sequence comprising (1) a CDR-H1 comprising the amino acid sequence SYAIS (SEQ ID NO: 85); (2) a CDR-H2 comprising the amino acid sequence set forth in GIIPIFGTANYAQKFQG (SEQ ID NO: 38); and (3) a CDR-H3 comprising the amino acid sequence VGATTSLYYYYGMDV (SEQ ID NO: 47).

In certain embodiments according to (or as applied to) any of the embodiments above, the light chain variable domain sequence comprises (1) a CDR-L1 comprising the amino acid sequence RASQSVSSSYLA (SEQ ID NO: 79); (2) a CDR-L2 comprising the amino acid sequence GASSRAT (SEQ ID NO: 56); and (3) a CDR-L3 comprising the amino acid sequence QQYGSSPGT (SEQ ID NO: 69); and a heavy chain variable domain sequence comprising (1) a CDR-H1 comprising the amino acid sequence SYSMN (SEQ ID NO: 28); (2) a CDR-H2 comprising the amino acid sequence SISSSSSYIYYADSVKG (SEQ ID NO: 35); and (3) a CDR-H3 comprising the amino acid sequence GIIVGPTDAFDI (SEQ ID NO: 48).

In certain embodiments according to (or as applied to) any of the embodiments above, the light chain variable domain sequence comprises (1) a CDR-L1 comprising the amino acid sequence RSSQSLYYRDGYTFLD (SEQ ID NO: 81); (2) a CDR-L2 comprising the amino acid sequence LSSKRDS (SEQ ID NO: 59); and (3) a CDR-L3 comprising the amino acid sequence MQGTHWPYT (SEQ ID NO: 70); and a heavy chain variable domain sequence comprising (1) a CDR-H1 comprising the amino acid sequence TYAMS (SEQ ID NO: 33); (2) a CDR-H2 comprising the amino acid sequence GISGSGGATHYADSVKG (SEQ ID NO: 39); and (3) a CDR-H3 comprising the amino acid sequence GLWFGEGY (SEQ ID NO: 49).

In certain embodiments according to (or as applied to) any of the embodiments above, the light chain variable domain sequence comprises (1) a CDR-L1 comprising the amino acid sequence RSSQSLLYSNGYNYLD (SEQ ID NO: 82); (2) a CDR-L2 comprising the amino acid sequence LGSNRAS SEQ ID NO: 54); and (3) a CDR-L3 comprising the amino acid sequence MQALQTPIT (SEQ ID NO: 71); and a heavy chain variable domain sequence comprising (1) a CDR-H1 comprising the amino acid sequence SYAIS (SEQ ID NO: 85); (2) a CDR-H2 comprising the amino acid sequence GIIPIFGTANYAQKFQG (SEQ ID NO: 38); and (3) a CDR-H3 comprising the amino acid sequence RDGSLGVGYYYMDF (SEQ ID NO: 50).

Provided herein is an anti-vascular endothelial growth factor receptor 2 (VEGFR2) antibody or antigen binding fragment that is a variant of an anti-VEGFR2 antibody comprising a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27), wherein the variant comprises at least one amino acid substitution in one or more of SEQ ID NOs: 22, 23, 24, 25, 26, and/or 27.

Also provided is an anti-vascular endothelial growth factor receptor 2 (VEGFR2) antibody or antigen binding fragment thereof that competitively inhibits the binding of a second anti-VEGFR2 antibody to VEGFR2, wherein the anti-VEGFR2 antibody comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27).

Also provided herein is an anti-vascular endothelial growth factor receptor 2 (VEGFR2) antibody or antigen binding fragment thereof that specifically binds to the same epitope of VEGFR2 as second anti-VEGFR2 antibody to VEGFR2, wherein the second anti-VEGFR2 antibody comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27).

In certain embodiments according to (or as applied to) any of the embodiments above, the antibody or antigen binding fragment thereof comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27), wherein the variant comprises at least one amino acid substitution in one or more of SEQ ID NOs: 22, 23, 24, 25, 26, and/or 27.

In certain embodiments according to (or as applied to) any of the embodiments above, the antibody or antigen binding fragment thereof comprises a light chain variable domain sequence comprising (1) a CDR-L1 comprising an amino acid sequence selected from consisting of SEQ ID NOs: 1 and 16; (2) a CDR-L2 comprising an amino acid sequence selected from consisting of SEQ ID NOs: 2, 7, and 8; and (3) a CDR-L3 comprising an amino acid sequence selected from consisting of SEQ ID NOs: 3, 9, and 12; and a heavy chain variable domain sequence comprising (1) a CDR-H1 comprising an amino acid sequence selected from consisting of SEQ ID NOs: 4, 13, 14, and 15; (2) a CDR-H2 comprising an amino acid sequence selected from consisting of SEQ ID NOs: 5, 7, 17, 18, 19, 20, and 21; (3) a CDR-H3 comprising an amino acid sequence selected from consisting of SEQ ID NOs: 6, 10, and 11.

In certain embodiments according to (or as applied to) any of the embodiments above, the antibody or antigen binding fragment thereof comprises a light chain variable domain sequence comprises (1) a CDR-L1 comprising the amino acid sequence QSLYYRDGYTF (SEQ ID NO: 1); (2) a CDR-L2 comprising the amino acid sequence LSS (SEQ ID NO: 2); and (3) a CDR-L3 comprising the amino acid sequence MQGTHWPYT (SEQ ID NO: 3); and a heavy chain variable domain sequence comprises (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

In certain embodiments according to (or as applied to) any of the embodiments above, the antibody or antigen binding fragment thereof comprises a light chain variable domain sequence comprising (1) a CDR-L1 comprising the amino acid sequence QSLYYRDGYTF (SEQ ID NO:1); (2) a CDR-L2 comprising the amino acid sequence QSS (SEQ ID NO: 7); and (3) a CDR-L3 comprising the amino acid sequence MQGTHWPYT (SEQ ID NO: 3); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

In certain embodiments according to (or as applied to) any of the embodiments above, the antibody or antigen binding fragment thereof comprises a light chain variable domain sequence comprising a (1) CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence LQGTHWPYT (SEQ ID NO: 9); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGL (SEQ ID NO: 10).

In certain embodiments according to (or as applied to) any of the embodiments above, the antibody or antigen binding fragment thereof comprises a light chain variable domain sequence comprising (1) a CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence FQGTHWPYT (SEQ ID NO: 12); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

In certain embodiments according to (or as applied to) any of the embodiments above, the antibody or antigen binding fragment thereof comprises a light chain variable domain sequence comprising (1) a CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence LQGTHWPYT (SEQ ID NO: 9); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

In certain embodiments according to (or as applied to) any of the embodiments above, the antibody or antigen binding fragment thereof comprises a light chain variable domain sequence comprising (1) a CDR-L1 comprising the amino acid sequence QSLYYRSGYTF (SEQ ID NO: 16); (2) a CDR-L2 comprising the amino acid sequence QSS (SEQ ID NO: 7); and (3) a CDR-L3 comprising the amino acid sequence MQGTHWPYT (SEQ ID NO: 3); and a heavy chain variable domain sequence comprising (1) a CDR-H1 comprising the amino acid sequence RFSFSTYA (SEQ ID NO: 15); (2) a CDR-H2 comprising the amino acid sequence ISGSGQAT (SEQ ID NO: 20); and (3) a CDR-H3 comprising the amino acid sequence KGLWFGEGY (SEQ ID NO: 6).

In certain embodiments according to (or as applied to) any of the embodiments above, the antibody or antigen binding fragment thereof comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGTT (SEQ ID NO: 21); and a CDR-H3 comprising the amino acid sequence KGLWFGEGY (SEQ ID NO: 6).

In certain embodiments according to (or as applied to) any of the embodiments above, the antibody or antigen binding fragment thereof comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGTT (SEQ ID NO: 21); and a CDR-H3 comprising the amino acid sequence KGLWFGEGL (SEQ ID NO: 10).

In certain embodiments according to (or as applied to) any of the embodiments above, the antibody comprises an Fc sequence of a human IgG. In certain embodiments according to (or as applied to) any of the embodiments above, the antigen binding fragment is selected from the group consisting of a Fab, Fab′, a F(ab)′ 2, a single-chain Fv (scFv), an Fv fragment, a diabody, and a linear antibody. In certain embodiments according to (or as applied to) any of the embodiments above, the antibody is a multi-specific antibody.

In certain embodiments according to (or as applied to) any of the embodiments above, the anti-VEGFR2 antibody or antigen binding fragment thereof according to any one of embodiments above is conjugated to a therapeutic agent. In certain embodiments according to (or as applied to) any of the embodiments above, the antibody or antigen binding fragment thereof is conjugated to a label. In certain embodiments according to (or as applied to) any of the embodiments above, the label is selected from the group consisting of a radioisotope, a fluorescent dye, and an enzyme.

Provided herein is an isolated nucleic acid molecule that encodes the anti-VEGFR2 antibody or antigen binding fragment thereof according to according to (or as applied to) any of the embodiments above. Also provided is an expression vector encoding the nucleic acid molecule according to (or as applied to) any of the embodiments above. Provided herein is a cell comprising the expression vector according to (or as applied to) any of the embodiments above. Provided herein is a method of producing an anti-VEGFR2 comprising culturing the cell according to (or as applied to) any of the embodiments above and recovering the anti-VEGFR2 from the cell culture.

Provided is a composition comprising the anti-VEGFR2 antibody or antigen binding fragment thereof according to (or as applied to) any of the embodiments above and a pharmaceutically acceptable carrier. Also provided is a method of detecting a VEGFR2 protein in sample from a patient by contacting the anti-VEGFR2 antibody or antigen binding fragment thereof according to (or as applied to) any of the embodiments above to the sample and detecting the anti-VEGFR2 antibody bound to the VEGFR2 protein. In certain embodiments according to (or as applied to) any of the embodiments above, the anti-VEGFR2 antibody or antigen binding fragment thereof is used an immunohistochemistry assay (IHC) or in an ELISA assay.

Provided herein is method of treating pathological condition characterized by excessive angiogenesis in a subject, comprising administering an effective amount of the composition according to (or as applied to) any of the embodiments above to the subject. In certain embodiments according to (or as applied to) any of the embodiments above, the pathological condition characterized by excessive angiogenesis is selected from the group consisting of cancer, an ocular disease, or inflammation. In certain embodiments according to (or as applied to) any of the embodiments above, the pathological condition characterized by excessive angiogenesis is cancer. In certain embodiments according to (or as applied to) any of the embodiments above, the cancer is colon cancer, colorectal cancer, gastric cancer, gastroesophageal cancer, bladder cancer, lung cancer, or solid tumor. In certain embodiments according to (or as applied to) any of the embodiments above, the subject is further administered a therapeutic agent selected from the group consisting of an anti-neoplastic agent, a chemotherapeutic agent, a growth inhibitory agent and a cytotoxic agent. In certain embodiments according to (or as applied to) any of the embodiments above, the cancer is non-small cell lung cancer (NSCLC).

### BRIEF DESCRIPTION OF THE DRAWINGS

FIG. 1 shows the results of ELISAs performed to compare the binding of anti-VEGFR2 antibodies V1, V9, V10, and 1121B (Ref A) to VEGFR2 domains 5-7.

FIG. 2 shows the results of ELISAs performed to assess the abilities of anti-VEGFR2 antibodies V1, V2, V3, V4, V5, V6, V7, V8, V9, V10, and 1121B (Ref A) to inhibit VEGF binding to VEGFR2.

FIG. 3 shows the results of assays performed to compare the abilities of V9, 1121B (Ref A) and 1121N (Ref B) to bind whole HUVEC cells.

FIG. 4 shows the results of assays performed to compare the effects of V1, V2, V6, V7, V8, V9, V10, and 1121B (Ref A) on HUVEC migration.

FIG. 5 shows the results of assays performed to compare the effects of V1, V2, V6, V7, V8, V9, V10, and 1121B (Ref A) on HUVEC survival.

FIG. 6 shows the results of assays performed to compare the effects of V1, V2, V6, V7, V8, V9, V10, and 1121B (Ref A) on HUVEC proliferation.

FIG. 7 shows the results of assays performed to compare the effects of V9 and 1121B (Ref A) on angiogenesis in vivo.

FIG. 8 depicts the results of an HCT-116 tumor xenograft assay measuring the abilities of V1, V9, V10, and 1121B (Ref A) to inhibit tumor growth.

FIG. 9 shows the results of ELISAs performed to compare the binding of anti-VEGFR2 antibodies V9, 29, 30, 32, 34, and 67 to VEGFR2.

FIG. 10A shows the results of ELISAs performed to compare the binding of anti-VEGFR2 antibodies V9/V9 (LC/HC), V9/29 (LC/HC), 34/V9 (LC/HC), 67/V9 (LC/HC), 29/34 (LC/HC), 34/34 (LC/HC), 67/34 (LC/HC), 1121B (Ref A), an anti-PDGFRa antibody, and ERBITUX (i.e., and anti-EGFR antibody) to VEGFR2. FIG. 10B shows the results of a second set of ELISAs performed to compare the binding of anti-VEGFR2 antibodies V9/V9 (LC/HC), V9/29 (LC/HC), 34/V9 (LC/HC), 67/V9 (LC/HC), 29/34 (LC/HC), 34/34 (LC/HC), 67/34 (LC/HC), 1121B (Ref A), an anti-PDGFRa antibody, and ERBITUX (i.e., and anti-EGFR antibody) to VEGFR2.

FIG. 11A shows the results of assays performed to assess the abilities of anti-VEGFR2 antibodies V9/V9 (LC/HC), V9/29 (LC/HC), 34/V9 (LC/HC), 67/V9 (LC/HC), 29/34 (LC/HC), 34/34 (LC/HC), 67/34 (LC/HC), 1121B (Ref A), 1211N (Ref B), and Avastin (i.e., anti-VEGF) to inhibit HUVEC proliferation in vitro. FIG. 11B shows the % inhibition of proliferation for each antibody tested in FIG. 11A.

FIG. 12A shows the results of assays performed to assess the abilities of anti-VEGFR2 antibodies V9/V9 (LC/HC), V9/29 (LC/HC), 34/V9 (LC/HC), 67/V9 (LC/HC), 29/34 (LC/HC), 34/34 (LC/HC), 67/34 (LC/HC), 1121B (Ref A), 1211N (Ref B), and Avastin (i.e., anti-VEGF) to inhibit HUVEC survival in vitro. FIG. 12B shows the % inhibition of survival for each antibody tested in FIG. 12A.

FIG. 13A shows the results of ELISAs performed to compare the binding of anti-VEGFR2 antibodies 34/80A (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 29/V9 (LC/HC), 34/34 (LC/HC), 67/34 (LC/HC), and 1121B (Ref A) to VEGFR2. FIG. 13B provides the results of FIG. 13A without 1121B (Ref A). FIG. 13C shows the results of a second set of ELISAs performed to compare the binding of anti-VEGFR2 antibodies 34/80A (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 29/V9 (LC/HC), 34/34 (LC/HC), 67/34 (LC/HC), and 1121B (Ref A) to VEGFR2. FIG. 13D provides the results of FIG. 13C without 1121B (Ref A).

FIG. 14A shows the results of ELISAs performed to compare the binding of anti-VEGFR2 antibodies 34/80A (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 29/V9 (LC/HC), 34/34 (LC/HC), 67/34 (LC/HC), and 1121B (Ref A) to VEGFR2. FIG. 14B provides the results of FIG. 14A without 1121B (Ref A). FIG. 14C shows the results of a second set of ELISAs s performed to compare the binding of anti-VEGFR2 antibodies 34/80A (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 29/V9 (LC/HC), 34/34 (LC/HC), 67/34 (LC/HC), and 1121B (Ref A) to VEGFR2. FIG. 14D provides the results of FIG. 14C without 1121B (Ref A).

FIG. 15 shows the results of assays performed to compare the abilities of 34/V9 (LC/HC), 109/109 (LC/HC), 110/110A (LC/HC), 110/110B (LC/HC), 1121B (Ref A) and 1121N (Ref B) to bind whole HUVEC cells.

FIG. 16A shows the results of assays performed to assess the abilities of anti-VEGFR2 antibodies V9/V9 (LC/HC), V9/29 (LC/HC), 34/V9 (LC/HC), 67/V9 (LC/HC), 29/34 (LC/HC), 34/34 (LC/HC), 67/34 (LC/HC), 1121B (Ref A), 1211N (Ref B), and Avastin (i.e., anti-VEGF) to inhibit HUVEC proliferation in vitro. FIG. 16B shows the % inhibition of proliferation for each antibody tested in FIG. 16A.

FIG. 17A shows the results of assays performed to assess the abilities of anti-VEGFR2 antibodies V9/V9 (LC/HC), V9/29 (LC/HC), 34/V9 (LC/HC), 67/V9 (LC/HC), 29/34 (LC/HC), 34/34 (LC/HC), 67/34 (LC/HC), 1121B (Ref A), 1211N (Ref B), and Avastin (i.e., anti-VEGF) to inhibit HUVEC survival in vitro. FIG. 17B shows the % inhibition of survival for each antibody tested in FIG. 17A.

FIG. 18A shows the results of ELISAs performed to assess the abilities of 34/V9 (LC/HC), 109/109 (LC/HC), 110A/110A (LC/HC), 110B/110B (LC/HC) 1121B (Ref A), 1121N (Ref B), and a control antibody (HLX02) to bind mouse VEGFR2. FIG. 18B shows the results of a duplicate set ELISAs.

FIG. 19A shows the results of assays performed to compare the abilities of 110B/110B (LC/HC) and 1121N (Ref B) to inhibit VEGFR2 phosphorylation. FIG. 19B provides quantitative results of FIG. 19A.

FIG. 20 shows the results of assays performed to compare the effects of V9, 34/80B (LC/HC), 34/86A (LC/HC), 34/86B (LC/HC), 29/88 (LC/HC), 109/109 (LC/HC), 110/110A (LC/HC), 110/110B (LC/HC), 1121N (Ref B), and Avastin on HUVEC migration.

FIG. 21 depicts the results of an HCT-116 tumor xenograft assay measuring the abilities of 34/V9 (LC/HC), 109/109 (LC/HC), 110A/110A (LC/HC), 110B/110B (LC/HC) 1121B (Ref A), and 1121N (Ref B) to inhibit tumor growth.

FIG. 22 depicts the results of an HCT-116 tumor xenograft assay measuring the abilities of 34/V9 (LC/HC), 109/109 (LC/HC), 110A/110A (LC/HC), 110B/110B (LC/HC) 1121B (Ref A), and 1121N (Ref B) to inhibit tumor growth.

FIG. 23A shows the results of stability tests performed on different formulations of 34/V9 (LC/HC). FIG. 23B shows the results of stability tests performed on different formulations of 110/110B (LC/HC).

FIG. 24A shows the results of experiments that were performed to determine whether antibody 110/110B is capable of binding VEGFR1. FIG. 24B shows the results of experiments that were performed to determine whether antibody 110/110B is capable of binding VEGFR3.

FIG. 25 shows the results of experiments that were performed to determine whether antibody 110/110B is capable of inhibiting VEGF-C stimulated human lymphatic endothelial cell (HLEC) proliferation.

FIG. 26A shows the results of experiments that were performed to determine whether antibody 110/110B is capable of inhibiting VEGF-C stimulated VEGFR2 phosphorylation. FIG. 26B provides the quantitated results of FIG. 26A.

FIG. 27A shows the results of experiments that were performed to determine whether antibody 110/110B is capable of inhibiting VEGF-C stimulated VEGFR3 phosphorylation. FIG. 27B provides the quantitated results of FIG. 26A.

FIG. 28 depicts the results of an NCI-H460 tumor xenograft assay measuring the ability of 110B/110B to inhibit tumor growth.

### DETAILED DESCRIPTION OF THE INVENTION

The present invention provides novel anti-vascular endothelial growth factor receptor 2 (VEGFR2) antibodies. Applicants have surprisingly found that, in contrast to other anti-VEGFR2 antibodies known in the art, the anti-VEGFR2 antibodies provided herein bind domains 5-7 of VEGR2. The anti-VEGFR2 antibodies described herein do not block binding of VEGF to VEGFR2, but are capable of inhibiting angiogenesis in vitro and in vivo.

Also provided are immunoconjugates, nucleic acids encoding the novel anti-VEGFR2 antibodies described herein, and compositions (such as pharmaceutical compositions). The invention also provides methods of using novel anti-VEGFR2 antibodies to detect VEGFR2 in a sample (such as an in vivo or ex vivo sample), compositions comprising such antibodies for use in treating cancer, and uses of such antibodies in the manufacture of a medicament for the treatment of cancer.

### Definitions

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening (e.g., the progression) of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods provided herein contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

The term “adjuvant therapy” refers to treatment given after the primary therapy, usually surgery. Adjuvant therapy for cancer or disease may include immune therapy, chemotherapy, radiation therapy, or hormone therapy.

The term “maintenance therapy” refers to scheduled retreatment that is given to help maintain a previous treatment's effects. Maintenance therapy is often given to help keep cancer in remission or prolong a response to a specific therapy regardless of disease progression.

The term “invasive cancer” refers to cancer that has spread beyond the layer of tissue in which it started into the normal surrounding tissues. Invasive cancers may or may not be metastatic.

The term “non-invasive cancer” refers to a very early cancer or a cancer that has not spread beyond the tissue of origin.

The term “progression-free survival” in oncology refers to the length of time during and after treatment that a cancer does not grow. Progression-free survival includes the amount of time patients have experienced a complete response or a partial response, as well as the amount of time patients have experienced stable disease.

The term “progressive disease” in oncology can refer to a tumor growth of more than 20 percent since treatment began—either due to an increase in mass or a spread in the tumor.

A “disorder” is any condition that would benefit from treatment with the antibody. For example, mammals who suffer from or need prophylaxis against abnormal VEGFR2 activity. This includes chronic and acute disorders or diseases including those pathological conditions which predispose the mammal to the disorder in question. Non-limiting examples of disorders to be treated herein include cancer (such as colon cancer, colorectal cancer, gastric cancer, gastroesophageal cancer, bladder cancer, lung cancer, and solid tumor) and pathological conditions characterized by excessive angiogenesis. Exemplary cancers and pathological conditions characterized by excessive angiogenesis that would benefit from treatment with an anti-VEGFR2 antibody provided by the invention are described in further detail elsewhere herein.

“Tumor”, as used herein, refers to all neoplastic cell growth and proliferation, whether malignant or benign, and all pre-cancerous and cancerous cells and tissues.

The term “antibody” is used in the broadest sense and specifically covers, for example, single monoclonal antibodies (including agonist, antagonist, and neutralizing antibodies), antibody compositions with polyepitopic specificity, polyclonal antibodies, single chain anti-antibodies, and fragments of antibodies (see below) as long as they specifically bind a native polypeptide and/or exhibit a biological activity or immunological activity of this invention. In certain embodiments, the antibody specifically binds to a protein, which binding can be inhibited by a monoclonal antibody of this invention (e.g., a deposited antibody of this invention, etc.). The phrase “functional fragment or analog” of an antibody is a compound having a qualitative biological activity in common with an antibody to which it is being referred. For example, a functional fragment or analog of an antibody of this invention can be one which can specifically bind to VEGFR2. In one embodiment, the antibody can prevent or substantially reduce the ability of VEGFR2 to induce cell proliferation.

An “isolated antibody” is one which has been identified and separated and/or recovered from a component of its natural environment. Contaminant components of its natural environment are materials which would interfere with diagnostic or therapeutic uses for the antibody, and can include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Ten and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, γ, ε, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR DEFINITIONS

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they can be synthesized uncontaminated by other antibodies. The modifier “monoclonal” is not to be construed as requiring production of the antibody by any particular method. For example, the monoclonal antibodies useful in the present invention can be prepared by the hybridoma methodology first described by Kohler et al. Nature. 256:495 (1975), or can be made using recombinant DNA methods in bacterial, eukaryotic animal or plant cells (see, e.g., U.S. Pat. No. 4,816,567). The “monoclonal antibodies” can also be isolated from phage antibody libraries using the techniques described in Clackson et al., Nature, 352:624-628 (1991), Marks et al., J. Mol. Biol., 222:581-597 (1991), and the Examples below, for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape, etc.), and human constant region sequences.

An “intact” antibody is one which comprises an antigen-binding site as well as a C_{L} and at least heavy chain constant domains, C_{H}1, C_{H}2 and C_{H}3. The constant domains can be native sequence constant domains (e.g. human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

The expression “linear antibodies” generally refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by di sulfides. The effector functions of antibodies are determined by sequences in the Fc region, which region is also the part recognized by Fc receptors (FcR) found on certain types of cells.

A “variant Fc region” comprises an amino acid sequence which differs from that of a native sequence Fc region by virtue of at least one “amino acid modification” as herein defined. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. In one embodiment, the variant Fc region herein will possess at least about 80% homology, at least about 85% homology, at least about 90% homology, at least about 95% homology or at least about 99% homology with a native sequence Fc region. According to another embodiment, the variant Fc region herein will possess at least about 80% homology, at least about 85% homology, at least about 90% homology, at least about 95% homology or at least about 99% homology with an Fc region of a parent polypeptide.

The term “Fc region-comprising polypeptide” refers to a polypeptide, such as an antibody or immunoadhesin (see definitions elsewhere herein), which comprises an Fc region. The C-terminal lysine (residue 447 according to the EU numbering system) of the Fc region may be removed, for example, during purification of the polypeptide or by recombinantly engineering the nucleic acid encoding the polypeptide. Accordingly, a composition comprising polypeptides, including antibodies, having an Fc region according to this invention can comprise polypeptides populations with all K447 residues removed, polypeptide populations with no K447 residues removed or polypeptide populations having a mixture of polypeptides with and without the K447 residue.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors; and B cell activation. A “native sequence Fc region” comprises an amino acid sequence identical to the amino acid sequence of an Fc region found in nature. Examples of Fc sequences are described in, for example, but not limited to, Kabat et al., Sequences of Immunological Interest. 5th Ed. Public Health Service, National Institutes of Health, Bethesda, Md. (1991)).

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

The terms “Fc receptor” or “FcR” are used to describe a receptor that binds to the Fc region of an antibody. In one embodiment, an FcR of this invention is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fc region (also referred to as “C1” of “H1” domain) usually extends from about amino acid 118 to about amino acid 215 (EU numbering system).

“Hinge region” is generally defined as stretching from Glu216 to Pro230 of human IgG1 (Burton, Molec. Immuno 1.22:161-206 (1985)). Hinge regions of other IgG isotypes may be aligned with the IgG1 sequence by placing the first and last cysteine residues forming inter-heavy chain S—S bonds in the same positions.

The “lower hinge region” of an Fc region is normally defined as the stretch of residues immediately C-terminal to the hinge region, i.e. residues 233 to 239 of the Fc region. In previous reports, FcR binding was generally attributed to amino acid residues in the lower hinge region of an IgG Fc region.

The “CH2 domain” of a human IgG Fc region (also referred to as “C2” of “H2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” or “H3” domain) comprises the stretch of residues C-terminal to a CH2 domain in an Fc region (i.e. from about amino acid residue 341 to the C-terminal end of an antibody sequence, typically at amino acid residue 446 or 447 of an IgG)

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays as herein disclosed, for example.

“C1q” is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

The term “binding domain” refers to the region of a polypeptide that binds to another molecule. In the case of an FcR, the binding domain can comprise a portion of a polypeptide chain thereof (e.g. the alpha chain thereof) which is responsible for binding an Fc region. One useful binding domain is the extracellular domain of an FcR alpha chain.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent Kd or IC50 value) than the parent polypeptide or a native sequence IgG Fc. According to some embodiments, the improvement in binding compared to a parent polypeptide is about 3 fold, preferably about 5, 10, 25, 50, 60, 100, 150, 200, up to 500 fold, or about 25% to 1000% improvement in binding. The polypeptide variant which “exhibits decreased binding” to an FcR, binds at least one FcR with lower affinity (e.g., higher apparent Kd or higher IC50 value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or in the Examples below may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates antibody-dependent cell-mediated cytotoxicity (ADCC) in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g. in an animal model etc. In one embodiment, the preferred variant is from about 5 fold to about 100 fold, e.g. from about 25 to about 50 fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164:4178-4184 (2000).

An “effective amount” of an anti-VEGFR2 antibody (or fragment thereof) or composition as disclosed herein is an amount sufficient to carry out a specifically stated purpose. An “effective amount” can be determined empirically and by known methods relating to the stated purpose.

The term “therapeutically effective amount” refers to an amount of an anti-VEGFR2 antibody (or fragment thereof) or composition as disclosed herein, effective to “treat” a disease or disorder in a mammal (aka patient). In the case of cancer, the therapeutically effective amount of the anti-VEGFR2 antibody (or fragment thereof) or composition as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the anti-VEGFR2 antibody (or fragment thereof) or composition as disclosed herein can prevent growth and/or kill existing cancer cells, it can be cytostatic and/or cytotoxic. In one embodiment, the therapeutically effective amount is a growth inhibitory amount. In another embodiment, the therapeutically effective amount is an amount that extends the survival of a patient. In another embodiment, the therapeutically effective amount is an amount that improves progression free survival of a patient.

A “growth inhibitory amount” of an anti-VEGFR2 antibody (or fragment thereof) or composition as disclosed herein of this invention is an amount capable of inhibiting the growth of a cell, especially tumor, e.g., cancer cell, either in vitro or in vivo. A “growth inhibitory amount” of a polypeptide, antibody, antagonist or composition of this invention for purposes of inhibiting neoplastic cell growth can be determined empirically and by known methods or by examples provided herein.

A “cytotoxic amount” of an anti-VEGFR2 antibody (or fragment thereof) or composition of this invention is an amount capable of causing the destruction of a cell, especially tumor, e.g., cancer cell, either in vitro or in vivo. A “cytotoxic amount” of an anti-VEGFR2 antibody (or fragment thereof) or composition of this invention for purposes of inhibiting neoplastic cell growth can be determined empirically and by methods known in the art.

A “growth inhibitory amount” of an anti-VEGFR2 antibody (or fragment thereof) or composition of this invention is an amount capable of inhibiting the growth of a cell, especially tumor, e.g., cancer cell, either in vitro or in vivo. A “growth inhibitory amount” of a an anti-VEGFR2 antibody (or fragment thereof) or composition of this invention for purposes of inhibiting neoplastic cell growth can be determined empirically and by known methods or by examples provided herein.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The term “detecting” is intended to include determining the presence or absence of a substance or quantifying the amount of a substance (such as VEGFR2). The term thus refers to the use of the materials, compositions, and methods of the present invention for qualitative and quantitative determinations. In general, the particular technique used for detection is not critical for practice of the invention.

For example, “detecting” according to the invention may include: observing the presence or absence of a VEGFR2 gene product, mRNA molecules, or aVEGFR2 polypeptide; a change in the levels of a VEGFR2 polypeptide or amount bound to a target; a change in biological function/activity of a VEGFR2 polypeptide. In some embodiments, “detecting” may include detecting wild type VEGFR2 levels (e.g., mRNA or polypeptide levels). Detecting may include quantifying a change (increase or decrease) of any value between 10% and 90%, or of any value between 30% and 60%, or over 100%, when compared to a control. Detecting may include quantifying a change of any value between 2-fold to 10-fold, inclusive, or more e.g., 100-fold.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects and embodiments of the invention described herein include “comprising,” “consisting,” and “consisting essentially of” aspects and embodiments.

All references cited herein, including patent applications and publications, are hereby incorporated by reference in their entirety.

### Anti-Vascular Endothelial Growth Factor Receptor 2 (VEGFR2) Antibodies

The present invention is based on the identification of novel antibodies that bind vascular endothelial growth factor receptor 2 (VEGFR2). The anti-VEGFR2 antibodies can be used in a variety of therapeutic and diagnostic methods. For example, the anti-VEGFR2 antibodies can be used alone or in combination with other agents in treating disease characterized by abnormal VEGFR2 expression or abnormal VEGFR2 activity, including, e.g., colon cancer, colorectal cancer, gastric cancer, gastroesophageal cancer, bladder cancer, lung cancer, solid tumor and pathological conditions characterized by excessive angiogenesis (such as those described elsewhere herein). The antibodies provided herein can also be used for detecting VEGFR2 protein in patients or patient samples by administering the anti-VEGFR2 antibodies to patients and detecting the anti-VEGFR2 antibody bound to the VEGFR2 protein in a sample from the patient (e.g., in vivo or ex vivo) or by contacting the anti-VEGFR2 antibodies with samples from patients and detecting qualitatively or quantitatively the anti-VEGFR2 antibody bound to the VEGFR2 protein.

Vascular endothelial growth factor receptor 2 or “VEGFR2” (also known as, e.g., KDR, “kinase insert domain receptor,” FLK1, “fetal liver kinase 1,” and CD309) is a member of the VEGFR family of receptors, a subfamily of three closely related receptor tyrosine kinases. VEGF receptors typically are class III receptor tyrosine kinases characterized by having several, typically 5 or 7, immunoglobulin-like loops in their amino terminal extracellular receptor ligand-binding domains (Kaipainen et al., J. Exp. Med., 178:2077-88 (1993)). VEGFR2, which promotes angiogenic and lymphangiogenic signaling, is the main VEGF receptor on endothelial cells. VEGFR2 is activated by binding of VEGFA, whereupon VEGFR2 undergoes dimerization and tyrosine autophosphorylation. Autophosphorylation leads to downstream direct activation of, e.g., SH2 domain-containing signal transduction molecules such as PLC-gamma, VRAP (VEGF Receptor-Associated Protein), and Sck, as well as indirect activation of Src and PI3K (Phosphatidylinositol 3-Kinase). It is generally believed that VEGFR2 is the main VEGF signal transducer that results in endothelial cell proliferation, migration, differentiation, tube formation, increase of vascular permeability, and maintenance of vascular integrity. Aberrant VEGFR2 expression or VEGFR2 activity is associated with many cancers (such as, e.g., colon cancer, colorectal cancer, gastric cancer, gastroesophageal cancer, bladder cancer, lung cancer, and solid tumor) and pathological conditions characterized by excessive angiogenesis (such as those described elsewhere herein)

An anti-VEGFR2 antibody is an antibody that binds to VEGFR2 with sufficient affinity and specificity. Preferably, an anti-VEGFR2 antibody provided herein (or the antigen-binding fragment thereof) can be used as a therapeutic agent in targeting and interfering with diseases or conditions wherein the VEGFR2 activity is involved. In certain embodiments, an anti-VEGFR2 antibody provided herein does not bind to other members of the VEGF receptor family, such as VEGFR1/FLT1 or VEGFR3/FLT4/PCL/Chy. In certain embodiments, an anti-VEGFR2 antibody provided herein also binds VEGFR3/FLT4/PCL/Chy. In certain embodiments, the anti-VEGFR2 antibody is a recombinant humanized anti-VEGFR2 monoclonal antibody. According to one embodiment, the anti-VEGFR2 antibody comprises the CDRs, the variable heavy chain region, and/or the variable light region of any one of the antibodies disclosed herein.

In certain embodiments, an anti-VEGFR2 antibody (or antigen binding fragment thereof) provided herein does not competitively inhibit the binding of a second anti-VEGFR2 antibody to human VEGFR2. In certain embodiments, the second anti-VEGFR2 antibody comprises a light chain variable domain sequence comprising a CDR-L1 comprising the amino acid sequence RASQSVSSYLA (SEQ ID NO: 73); a CDR-L2 comprising the amino acid sequence DSSNRAT (SEQ ID NO: 52); and a CDR-L3 comprising the amino acid sequence LQHNTFPPT (SEQ ID NO: 61); and a heavy chain variable domain sequence comprising a CDR-H1 comprising the amino acid sequence SYSMN (SEQ ID NO: 28); a CDR-H2 comprising the amino acid sequence SISSSSSYIYYADSVKG (SEQ ID NO: 35); and a CDR-H3 comprising the amino acid sequence VTDAFDI (SEQ ID NO: 41). In certain embodiments, the second anti-VEGFR2 antibody is 1121B, described in US2009/0306348, the contents of which are incorporated herein by reference in their entirety. In certain embodiments, the second anti-VEGFR2 antibody comprises a light chain variable domain sequence comprising a CDR-L1 comprising the amino acid sequence RASQGIDNWLG (SEQ ID NO: 74); a CDR-L2 comprising the amino acid sequence DASNLDT (SEQ ID NO: 53); and a CDR-L3 comprising the amino acid sequence QQAKAFPPT (SEQ ID NO: 62); and a heavy chain variable domain sequence comprising a CDR-H1 comprising the amino acid sequence SYSMN (SEQ ID NO: 28); a CDR-H2 comprising the amino acid sequence SISSSSSYIYYADSVKG (SEQ ID NO: 35); and a CDR-H3 comprising the amino acid sequence VTDAFDI (SEQ ID NO: 41). In certain embodiments, the second anti-VEGFR2 antibody is 1121N, described in U.S. Pat. No. 7,498,414, the contents of which are incorporated herein by reference in their entirety.

In certain embodiments, an anti-VEGFR2 antibody (or antigen binding fragment thereof) provided herein does not bind a domain of VEGFR2 bound by a second anti-VEGFR2 antibody. In certain embodiments, the second anti-VEGFR2 antibody comprises a light chain variable domain sequence comprising a CDR-L1 comprising the amino acid sequence RASQSVSSYLA (SEQ ID NO: 73); a CDR-L2 comprising the amino acid sequence DSSNRAT (SEQ ID NO: 52); and a CDR-L3 comprising the amino acid sequence LQHNTFPPT (SEQ ID NO: 61); and a heavy chain variable domain sequence comprising a CDR-H1 comprising the amino acid sequence SYSMN (SEQ ID NO: 28); a CDR-H2 comprising the amino acid sequence SISSSSSYIYYADSVKG (SEQ ID NO: 35); and a CDR-H3 comprising the amino acid sequence VTDAFDI (SEQ ID NO: 41). In certain embodiments, the second anti-VEGFR2 antibody comprises a light chain variable domain comprising the amino acid sequence SEQ ID NO: 104 and a heavy chain variable domain comprising the amino acid sequence SEQ ID NO: 106. In certain embodiments, the second anti-VEGFR2 antibody is 1121B, described in US2009/0306348. In certain embodiments, the second anti-VEGFR2 antibody comprises a light chain variable domain sequence comprising a CDR-L1 comprising the amino acid sequence RASQGIDNWLG (SEQ ID NO: 74); a CDR-L2 comprising the amino acid sequence DASNLDT (SEQ ID NO: 53); and a CDR-L3 comprising the amino acid sequence QQAKAFPPT (SEQ ID NO: 62); and a heavy chain variable domain sequence comprising a CDR-H1 comprising the amino acid sequence SYSMN (SEQ ID NO: 28); a CDR-H2 comprising the amino acid sequence SISSSSSYIYYADSVKG (SEQ ID NO: 35); and a CDR-H3 comprising the amino acid sequence VTDAFDI (SEQ ID NO: 41). In certain embodiments, the second anti-VEGFR2 antibody comprises a light chain variable domain comprising the amino acid sequence SEQ ID NO: 105 and a heavy chain variable domain comprising the amino acid sequence SEQ ID NO: 106. In certain embodiments, the second anti-VEGFR2 antibody is 1121N, described in U.S. Pat. No. 7,498,414.

The amino acid sequences of SEQ ID NO:s 104-106 are provided below:

(SEQ ID NO: 104)
EIVMTQSPATLSLSPGERATLSCRASQSVSSYLAWYQQKPGQAPRLLIYD
SSNRATGIPARFSGSGSGTDFTLTISSLEPEDFATYYCLQHNTFPPTFGQ
GTKVEIKRTVAAPSVFIFPPSDEQLKSGTASVVCLLNNFYPREAKVQWKV
DNALQSGN
(SEQ ID NO: 105)
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIKGTVAAPSVFIFPPSDEQLKSGTASVVCLLNNFYPREAKVQWKV
DNALQSGN
(SEQ ID NO: 106)
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSSASTKGPSVFPLAPSSKSTSGGTAALGCLVKDYFP
EPVT

In certain embodiments, an anti-VEGFR2 antibody provided herein does not block the binding of VEGFR2 to VEGF.

In certain embodiments, an anti-VEGFR2 antibody provided herein binds to domains 5-7 of VEGFR2.

In certain embodiments, an anti-VEGFR2 antibody provided herein binds to whole HUVEC cells, as determined via flow cytometry, as described in the Examples below.

In certain embodiments, an anti-VEGFR2 antibody provided herein inhibits tube formation in a human umbilical vein endothelial cell (HUVEC) tube formation assay (such as the HUVEC tube formation assay described in the Examples below). In certain embodiments, an anti-VEGFR2 antibody provided herein inhibits HUVEC migration in a HUVEC migration assay (such as the HUVEC migration assay described in the Examples below). In certain embodiments, an anti-VEGFR2 antibody provided herein does not inhibit HUVEC migration in a HUVEC migration assay (such as the HUVEC migration assay described in the Examples below). In certain embodiments, an anti-VEGFR2 antibody provided herein inhibits HUVEC survival in a HUVEC survival assay (such as the HUVEC survival assay described in the Examples below). In certain embodiments, an anti-VEGFR2 antibody provided herein inhibits HUVEC proliferation in a HUVEC proliferation assay (such as the HUVEC proliferation assay described in the Examples below).

In certain embodiments, an anti-VEGFR2 antibody provided herein does not inhibit angiogenesis in an in vitro assay (such as a HUVEC sprouting assay, as described in the Examples below). In certain embodiments, an anti-VEGFR2 antibody provided herein inhibits angiogenesis in an in vivo assay (such as a HUVEC matrigel plug assay, as described in the Examples below). In certain embodiments, an anti-VEGFR2 antibody provided herein does not inhibit angiogenesis in an in vitro assay (such as a HUVEC sprouting assay, as described in the Examples below), but inhibits angiogenesis in an in vivo assay (such as a HUVEC matrigel plug assay, as described in the Examples below).

In certain embodiments, the anti-VEGFR2 antibody (or antigen binding fragment thereof) comprises a light chain variable domain sequence comprising a CDR-L1 comprising the amino acid sequence RASQNIASYLN (SEQ ID NO: 76) or RASQSVS-S/N-S/N-YL-G/A (SEQ ID NO: 83) or TRSRGSIASSYVQ (SEQ ID NO: 80) or RSSQSL-L/V/Y-H/Y-G/S/R-D/N-G-N/K/Y-N/T-Y/F-LD (SEQ ID NO: 84); a CDR-L2 comprising the amino acid sequence L/A/G/K/E-G/A/V/N/S-S/D-N/S/Q/K-R/L-A/K/D/P-S/T (SEQ ID NO: 60); and a CDR-L3 comprising the amino acid sequence M/Q-Q/S-A/S/R/G/Y-L/Y/S/A/D/G/T-Q/S/N/H/F-T/I/W/S-P/T-Y/L/P/V/G/I-T/V (SEQ ID NO: 72); and a heavy chain variable domain sequence comprising a CDR-H1 comprising the amino acid sequence T/S-Y-Y/G/A/S-M/I-H/N/S (SEQ ID NO: 34); a CDR-H2 comprising the amino acid sequence I/V/G/S-I-N/S/I-P/Y/S/G-S/D/I-G/F/S-G/S-S/N/T/Y/A-T/K/A/I-S/Y/N/H-YA-Q/D-K/S-F/V-K/Q-G (SEQ ID NO: 40); and a CDR-H3 comprising the amino acid sequence GLWFGEGY (SEQ ID NO: 49) or ESYGGQFDY (SEQ ID NO: 43) or DLVVPAATLDY (SEQ ID NO: 42) or D/G-F/I-Y/I-E/V-A/G-G/P-G/T-W/D-Y/A-FD-L/I (SEQ ID NO: 51) or RDGSLGVGYYYMDF (SEQ ID NO: 50) or VGATTSLYYYYGMDV (SEQ ID NO: 47) or DGFGLAVAGPYWYFDL (SEQ ID NO: 44) or PTRSRDFWSGLGYYYYMDV (SEQ ID NO: 45).

In certain embodiments, the anti-VEGFR2 antibody (or antigen binding fragment thereof) comprises a light chain variable domain sequence comprising a CDR-L1 comprising an amino acid sequence selected from the group consisting of SEQ ID NOs: 75-82; a CDR-L2 comprising an amino acid sequence selected from the group consisting of SEQ ID NOs: 54-59; and a CDR-L3 comprising an amino acid sequence selected from the group consisting of SEQ ID NOs: 63-71; and a heavy chain variable domain sequence comprising a CDR-H1 comprising an amino acid sequence selected from the group consisting of SEQ ID NOs: 29-33; a CDR-H2 comprising an amino acid sequence selected from the group consisting of SEQ ID NOs: 35-39 and 125; and a CDR-H3 comprising an amino acid sequence selected from the group consisting of SEQ ID NOs: 42-50.

In certain embodiments, the anti-VEGFR2 antibody (or antigen binding fragment thereof) comprises a light chain variable domain sequence comprising a CDR-L1 comprising the amino acid sequence RSSQSLLHGNGNNYLD (SEQ ID NO: 75); a CDR-L2 comprising the amino acid sequence LGSNRAS (SEQ ID NO: 54); and a CDR-L3 comprising the amino acid sequence MQALQTPYT (SEQ ID NO: 63); and a heavy chain variable domain sequence comprising a CDR-H1 comprising the amino acid sequence TYYMH (SEQ ID NO: 29); a CDR-H2 comprising the amino acid sequence IINPSGGSTSYAQKFQG (SEQ ID NO: 36); and a CDR-H3 comprising the amino acid sequence DLVVPAATLDY (SEQ ID NO: 42).

In certain embodiments, the anti-VEGFR2 antibody (or antigen binding fragment thereof) comprises a light chain variable domain sequence comprising a CDR-L1 comprising the amino acid sequence RASQNIASYLN (SEQ ID NO: 76); a CDR-L2 comprising the amino acid sequence AASSLKS (SEQ ID NO: 55); and a CDR-L3 comprising the amino acid sequence QQSYSIPYT (SEQ ID NO: 64); and a heavy chain variable domain sequence comprising a CDR-H1 comprising the amino acid sequence SYGMH (SEQ ID NO: 30); a CDR-H2 comprising the amino acid sequence VISYDGSNKYYADSVKG (SEQ ID NO: 37); and a CDR-H3 comprising the amino acid sequence ESYGGQFDY (SEQ ID NO: 43).

In certain embodiments, the anti-VEGFR2 antibody (or antigen binding fragment thereof) comprises a light chain variable domain sequence comprising a CDR-L1 comprising the amino acid sequence RASQSVSNNYLG (SEQ ID NO: 77); a CDR-L2 comprising the amino acid sequence GASSRAT (SEQ ID NO: 56); and a CDR-L3 comprising the amino acid sequence QQRSNWPLT (SEQ ID NO: 65); and a heavy chain variable domain sequence comprising a CDR-H1 comprising the amino acid sequence SYAMH (SEQ ID NO: 31); a CDR-H2 comprising the amino acid sequence VISYDGSNKYYADSVKG (SEQ ID NO: 37); and a CDR-H3 comprising the amino acid sequence DGFGLAVAGPYWYFDL (SEQ ID NO: 44).

In certain embodiments, the anti-VEGFR2 antibody (or antigen binding fragment thereof) comprises a light chain variable domain sequence comprising a CDR-L1 comprising the amino acid sequence RSSQSLVYSDGKTYLD (SEQ ID NO: 78); a CDR-L2 comprising the amino acid sequence KVSNRDS (SEQ ID NO: 57); and a CDR-L3 comprising the amino acid sequence MQGAHWPPT (SEQ ID NO: 66); and a heavy chain variable domain sequence comprising a CDR-H1 comprising the amino acid sequence SYAIS (SEQ ID NO: 85); a CDR-H2 comprising the amino acid sequence GIIPIFGTANYAQKFQG (SEQ ID NO: 38); and a CDR-H3 comprising the amino acid sequence PTRSRDFWSGLGYYYYMDV (SEQ ID NO: 45).

In certain embodiments, the anti-VEGFR2 antibody (or antigen binding fragment thereof) comprises a light chain variable domain sequence comprising a CDR-L1 comprising the amino acid sequence RASQSVSSSYLA (SEQ ID NO: 79); a CDR-L2 comprising the amino acid sequence GASSRAT (SEQ ID NO: 56); and a CDR-L3 comprising the amino acid sequence QQRSNWPPT (SEQ ID NO: 67); and a heavy chain variable domain sequence comprising a CDR-H1 comprising the amino acid sequence SYGMH (SEQ ID NO: 30); a CDR-H2 comprising the amino acid sequence VISYDGSNKHYADSVKG (SEQ ID NO: 125); and a CDR-H3 comprising the amino acid sequence DFYEAGGWYFDL (SEQ ID NO: 46).

In certain embodiments, the anti-VEGFR2 antibody (or antigen binding fragment thereof) comprises a light chain variable domain sequence comprising a CDR-L1 comprising the amino acid sequence TRSRGSIASSYVQ (SEQ ID NO: 80); a CDR-L2 comprising the amino acid sequence ENDQRPS (SEQ ID NO: 58); and a CDR-L3 comprising the amino acid sequence QSYDFSTVV (SEQ ID NO: 68); and a heavy chain variable domain sequence comprising a CDR-H1 comprising the amino acid sequence SYAIS (SEQ ID NO: 85); a CDR-H2 comprising the amino acid sequence GIIPIFGTANYAQKFQG (SEQ ID NO: 38); and a CDR-H3 comprising the amino acid sequence VGATTSLYYYYGMDV (SEQ ID NO: 47).

In certain embodiments, the anti-VEGFR2 antibody (or antigen binding fragment thereof) comprises a light chain variable domain sequence comprising a CDR-L1 comprising the amino acid sequence RASQSVSSSYLA (SEQ ID NO: 79); a CDR-L2 comprising the amino acid sequence GASSRAT (SEQ ID NO: 56); and a CDR-L3 comprising the amino acid sequence QQYGSSPGT (SEQ ID NO: 69); and a heavy chain variable domain sequence comprising a CDR-H1 comprising the amino acid sequence SYSMN (SEQ ID NO: 28); a CDR-H2 comprising the amino acid sequence SISSSSSYIYYADSVKG (SEQ ID NO: 35); and a CDR-H3 comprising the amino acid sequence GIIVGPTDAFDI (SEQ ID NO: 48).

In certain embodiments, the anti-VEGFR2 antibody (or antigen binding fragment thereof) comprises a light chain variable domain sequence comprising a CDR-L1 comprising the amino acid sequence RSSQSLYYRDGYTFLD (SEQ ID NO: 81); a CDR-L2 comprising the amino acid sequence LSSKRDS (SEQ ID NO: 59); and a CDR-L3 comprising the amino acid sequence MQGTHWPYT (SEQ ID NO: 70); and a heavy chain variable domain sequence comprising a CDR-H1 comprising the amino acid sequence TYAMS (SEQ ID NO: 33); a CDR-H2 comprising the amino acid sequence GISGSGGATHYADSVKG (SEQ ID NO: 39); and a CDR-H3 comprising the amino acid sequence GLWFGEGY (SEQ ID NO: 49).

In certain embodiments, the anti-VEGFR2 antibody (or antigen binding fragment thereof) comprises a light chain variable domain sequence comprising a CDR-L1 comprising the amino acid sequence RSSQSLLYSNGYNYLD (SEQ ID NO: 82); a CDR-L2 comprising the amino acid sequence LGSNRAS (SEQ ID NO: 54); and a CDR-L3 comprising the amino acid sequence MQALQTPIT (SEQ ID NO: 71); and a heavy chain variable domain sequence comprising a CDR-H1 comprising the amino acid sequence SYAIS (SEQ ID NO: 85); a CDR-H2 comprising the amino acid sequence GIIPIFGTANYAQKFQG (SEQ ID NO: 38); and a CDR-H3 comprising the amino acid sequence RDGSLGVGYYYMDF (SEQ ID NO: 50).

The sequences of the CDRs noted herein are provided in Table 2 below.

TABLE 2

| | |
|---|---|
| SEQ ID NO: 28 | SYSMN |
| SEQ ID NO: 29 | TYYMH |
| SEQ ID NO: 30 | SYGMH |
| SEQ ID NO: 31 | SYAMH |
| SEQ ID NO: 32 | SYSMN |
| SEQ ID NO: 33 | TYAMS |
| SEQ ID NO: 35 | SISSSSSYIYYADSVKG |
| SEQ ID NO: 36 | IINPSGGSTSYAQKFQG |
| SEQ ID NO: 37 | VISYDGSNKYYADSVKG |
| SEQ ID NO: 38 | GIIPIFGTANYAQKFQG |
| SEQ ID NO: 39 | GISGSGGATHYADSVKG |
| SEQ ID NO: 42 | DLVVPAATLDY |
| SEQ ID NO: 43 | ESYGGQFDY |
| SEQ ID NO: 44 | DGFGLAVAGPYWYFDL |
| SEQ ID NO: 45 | PTRSRDFWSGLGYYYYMDV |
| SEQ ID NO: 46 | DFYEAGGWYFDL |
| SEQ ID NO: 47 | VGATTSLYYYYGMDV |
| SEQ ID NO: 48 | GIIVGPTDAFDI |
| SEQ ID NO: 49 | GLWFGEGY |
| SEQ ID NO: 50 | RDGSLGVGYYYMDF |
| SEQ ID NO: 54 | LGSNRAS |
| SEQ ID NO: 55 | AASSLKS |
| SEQ ID NO: 125 | VISYDGSNKHYADSVKG |
| SEQ ID NO: 56 | GASSRAT |
| SEQ ID NO: 57 | KVSNRDS |
| SEQ ID NO: 58 | ENDQRPS |
| SEQ ID NO: 59 | LSSKRDS |
| SEQ ID NO: 63 | MQALQTPYT |
| SEQ ID NO: 64 | QQSYSIPYT |
| SEQ ID NO: 65 | QQRSNWPLT |
| SEQ ID NO: 66 | MQGAHWPPT |
| SEQ ID NO: 67 | QQRSNWPPT |
| SEQ ID NO: 68 | QSYDFSTVV |
| SEQ ID NO: 69 | QQYGSSPGT |
| SEQ ID NO: 70 | MQGTHWPYT |
| SEQ ID NO: 71 | MQALQTPIT |
| SEQ ID NO: 75 | RSSQSLLHGNGNNYLD |
| SEQ ID NO: 76 | RASQNIASYLN |
| SEQ ID NO: 77 | RASQSVSNNYLG |
| SEQ ID NO: 78 | RSSQSLVYSDGKTYLD |
| SEQ ID NO: 79 | RASQSVSSSYLA |
| SEQ ID NO: 80 | TRSRGSIASSYVQ |
| SEQ ID NO: 81 | RSSQSLYYRDGYTFLD |
| SEQ ID NO: 82 | RSSQSLLYSNGYNYLD |
| SEQ ID NO: 85 | SYAIS |

In certain embodiments, the anti-VEGFR2 antibody (or antigen binding fragment thereof) comprises a light chain variable domain sequence selected from the group consisting of SEQ ID NOs 86-94 and a heavy chain variable domain sequence selected from the group consisting of SEQ ID NOs 95-103.

The amino acid sequences of SEQ ID NOs: 86-103 are provided below.

(SEQ ID NO: 86)
DVVMTQSPLSLPVTPGESASISCRSSQSLLHGNGNNYLDWYLQKPGQSPQLLIYLGSNRASGVP
DRFSGSGSGTDFTLQISRVEPEDVGVYYCMQALQTPYTFGQGTKLEIKRTVAAPSVFIFPPSDE
QLKSGTASVVCLLNNFYPREAKVQWKVDNALQSGN
(SEQ ID NO: 87)
DVVMTQSPSSLSASVGDRVTVTCRASQNIASYLNWYQQKPGKAPKLLIYAASSLKSGVPSRFSG
SGSGRDFTLTISSLQPEDFAAYYCQQSYSIPYTFGQGTKVEIKRTVAAPSVFIFPPSDEQLKSG
TASVVCLLNNFYPREAKVQWKVDNALQLGN
(SEQ ID NO: 88)
EIVLTQSPGTLSLSPGERATLSCRASQSVSNNYLGWYQQKPGQAPRLLIYGASSRATGVPARFS
GSGSGTDFTLTISSLEPEDFAVYYCQQRSNWPLTFGGGTKVEIKRTVAAPSVFIFPPSDEQLKS
GTASVVCLLNNFYPREAKVQWKVDNALQSGN
(SEQ ID NO: 89)
DVVMTQSPLSLSVTPGEPASISCRSSQSLVYSDGKTYLDWFLQRPGQSPRRLIYKVSNRDSGVS
DRFSGSGSGTDFTLKISRVEAEDVGVYYCMQGAHWPPTFGQGTRVEIKRTVAAPSVFIFPPSDE
QLKSGTASVVCLLNNFYPREAKVQWKVDNALQSGN
(SEQ ID NO: 90)
EIVLTQSPGTLSLSPGERATLSCRASQSVSSSYLAWYQQKPGQAPRLLIYGASSRATGIPARFS
GSGSGTDFTLTISSLEPEDFAVYYCQQRSNWPPTFGPGTKVDIKRTVAAPSVFIFPPSDEQLKS
GTASVVCLLNNFYPREAKVQWKVDNALQSGN
(SEQ ID NO: 91)
NFMLTQPHSVSESPGKTVTVSCTRSRGSIASSYVQWYQQRPGRSPTNVIYENDQRPSGVPTRFS
GSVDRSSNSASLTISGLETEDEADYYCQSYDFSTVVFGGGTKLTVLSQPKAAPSVTLFPPSSEE
LQANKATLVCLISDFYPGAVTVAWKADSSPVKAG
(SEQ ID NO: 92)
EIVLTQSPGTLSLSPGERATLSCRASQSVSSSYLAWYQQKPGQAPRLLIYGASSRATGIPDRFS
VSGSGTDFTLTISRLEPEDFAVYYCQQYGSSPGTFGQGTKVEIKRTVAAPSVFIFPPSDEQLKS
GTASVVCLLNNFYPREAKVQWKVDNALQSGN
(SEQ ID NO: 93)
DVVMTQSPLSLPVTLGQPASISCRSSQSLYYRDGYTFLDWYVQKPGQSPQLLIYLSSKRDSGVP
DRISGSGSGTDFTLRISRVEAEDVGVYYCMQGTHWPYTFGQGTKLEIKRTVAAPSVFIFPPSDE
QLKSGTASVVCLLNNFYPREAKVQWKVDNALQSGN
(SEQ ID NO: 94)
DVVMTQSPLSLAVTPGEPASISCRSSQSLLYSNGYNYLDWYLQKPGQSPQLLIYLGSNRASGVP
DRFSGSGSGTDFTLKISRVEAEDVGVYYCMQALQTPITFGPGTKVDIKRTVAAPSVFIFPPSDE
QLKSGTASVVCLLNNFYPREAKVQWKVDNALQSGN
(SEQ ID NO: 95)
EVQLVQSGAEVKKPGASVKVSCRASGFSFTTYYMHWVRQAPGQGLEWMGIINPSGGSTSYAQKF
QGRVTMTRDTSTSTVYMELSSLRSEDTAVYYCARDLVVPAATLDYWGQGTLVTVSSASTKGPSV
FPLAPSSKSTSGGTAALGCLVKDYFPEPVT
(SEQ ID NO: 96)
EVQLVQTGGGAVQPGRSLRLSCAATGFTFSSYGMHWVRQAPGKGLEWVAVISYDGSNKYYADSV
KGRFTISRDNAKNTLYLQMNSLRAEDTAVYYCARESYGGQFDYWGPGTLVTVSSASTKGPSVFP
LAPSSKSTSGGTAALGCLVKDYFPEPVT
(SEQ ID NO: 97)
EVQLVQSGGGVVQPGRSLRLSCAASGFTFSSYAMHWVRQAPGKGLEWVAVISYDGSNKYYADSV
KGRFTISRDNSKNTLYLQMNSLRAEDTAVYYCARDGFGLAVAGPYWYFDLWGRGTLVTVSSAST
KGPSVFPLAPSSKSTSGGTAALGCLVKDYFPEPVT
(SEQ ID NO: 98)
EVQLVQSGAEVKKPGSSVKVSCKASGGTFSSYAISWVRQAPGQGLEWMGGIIPIFGTANYAQKF
QGRVTITADESTSTAYMELSSLRSEDTAVYYCAGPTRSRDFWSGLGYYYYMDVWGKGTTVTVSS
ASTKGPSVFPLAPSSKSTSGGTAALGCLVKDYFPEPVT
(SEQ ID NO: 99)
EVQLVQSGGGVVQPGRSLRLSCAASGFTFSSYGMHWVRQAPGKGLEWVAVISYDGSNKHYADSV
KGRFTISRDNSKNTLYLQMNSLRAEDTAVYYCARDFYEAGGWYFDLWGRGTLVTVSSASTKGPS
VFPLAPSSKSTSGGTAALGCLVKDYFPEPVT
(SEQ ID NO: 100)
EVQLVQSGAEVKKPGSSVKVSCKASGGTFSSYAISWVRQAPGQGLEWMGGIIPIFGTA
YAQKFQGRVTITADESTSTAYMELSSLRSEDTAVYYCARVGATTSLYYYYGMDVWGQGTTVTVS
SASTKGPSVFPLAPSSKSTSGGTAALGCLVKDYFPEPVT
(SEQ ID NO: 101)
EVQLVQSGGGLVKPGGSLRLSCAASGFTFSSYSMNWVRQAPGKGLEWVSSISSSSSYIYYADSV
KGRFTISRDNAKNSLYLQMNSLRAEDTAVYYCARGIIVGPTDAFDIWGQGTTVTVSSASTKGPS
VFPLAPSSKSTSGGTAALGCLVKDYFPEPVT
(SEQ ID NO: 102)
QVQLVQSGGGLVQPGGSLRLSCAASGFSFSTYAMSWVRQAPGKGLEWVSGISGSGGATHYADSV
KGRFTISRDNSKNTVNLQMNSLRAEDTAVYYCAKGLWFGEGYWGQGTLVTVSSASTKGPSVFPL
APSSKSTSGGTAALGCLVKDYFPEPVT
(SEQ ID NO: 103)
EVQLVQSGAEVKKPGSSVKVSCKASGGTFSSYAISWVRQAPGQGLEWMGGIIPIFGTANYAQKF
QGRVTITADESTSTAYMELSSLRSEDTAVYYCARDGSLGVGYYYMDFWGKGTTVTVSSASTKGP
SVFPLAPSSKSTSGGTAALGCLVKDYFPEPVT

In certain embodiments, the anti-VEGFR2 antibody or antigen binding fragment thereof comprises a light chain variable domain comprising the amino acid sequence set forth in SEQ ID NO: 86 and a heavy chain variable domain comprising the amino acid sequence set forth in SEQ ID NO: 95. In certain embodiments, the anti-VEGFR2 antibody or antigen binding fragment thereof comprises a light chain variable domain comprising the amino acid sequence set forth in SEQ ID NO: 87 and a heavy chain variable domain comprising the amino acid sequence set forth in SEQ ID NO: 96. In certain embodiments, the anti-VEGFR2 antibody or antigen binding fragment thereof comprises a light chain variable domain comprising the amino acid sequence set forth in SEQ ID NO: 88 and a heavy chain variable domain comprising the amino acid sequence set forth in SEQ ID NO: 97. In certain embodiments, the anti-VEGFR2 antibody or antigen binding fragment thereof comprises a light chain variable domain comprising the amino acid sequence set forth in SEQ ID NO: 89 and a heavy chain variable domain comprising the amino acid sequence set forth in SEQ ID NO: 98. In certain embodiments, the anti-VEGFR2 antibody or antigen binding fragment thereof comprises a light chain variable domain comprising the amino acid sequence set forth in SEQ ID NO: 90 and a heavy chain variable domain comprising the amino acid sequence set forth in SEQ ID NO: 99. In certain embodiments, the anti-VEGFR2 antibody or antigen binding fragment thereof comprises a light chain variable domain comprising the amino acid sequence set forth in SEQ ID NO: 91 and a heavy chain variable domain comprising the amino acid sequence set forth in SEQ ID NO: 100. In certain embodiments, the anti-VEGFR2 antibody or antigen binding fragment thereof comprises a light chain variable domain comprising the amino acid sequence set forth in SEQ ID NO: 92 and a heavy chain variable domain comprising the amino acid sequence set forth in SEQ ID NO: 101. In certain embodiments, the anti-VEGFR2 antibody or antigen binding fragment thereof comprises a light chain variable domain comprising the amino acid sequence set forth in SEQ ID NO: 93 and a heavy chain variable domain comprising the amino acid sequence set forth in SEQ ID NO: 102. In certain embodiments, the anti-VEGFR2 antibody or antigen binding fragment thereof comprises a light chain variable domain comprising the amino acid sequence set forth in SEQ ID NO: 94 and a heavy chain variable domain comprising the amino acid sequence set forth in SEQ ID NO: 103.

The heavy and light chain variable domains are combined in all possible pair-wise combinations to generate a number of anti-VEGFR2 antibodies.

In certain embodiments, the anti-VEGFR2 antibody (or antigen binding fragment thereof) competitively inhibits the binding of a second anti-VEGFR2 antibody to human VEGFR2. In some embodiments, the second anti-VEGFR2 antibody is variant of an anti-VEGFR2 antibody comprising a light chain variable domain sequence comprising a CDR-L1 comprising the amino acid sequence QSLYYRDGYTF (SEQ ID NO: 1); a CDR-L2 comprising the amino acid sequence LSS (SEQ ID NO: 2); and a CDR-L3 comprising the amino acid sequence MQGTHWPYT (SEQ ID NO: 3); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6), wherein the variant comprises at least one amino acid substitution in one or more of SEQ ID NOs: 1, 2, 3, 4, 5, and/or 6. In some embodiments the variant comprises at least 1, at least 2, at least 3, at least 4, at least 5, at least 6, at least 7, at least 8, at least 9, or at least 10 amino acid substitutions in one or more of SEQ ID NOs: 1, 2, 3, 4, 5, and/or 6. In certain embodiments, the amino acid substitution(s) are conservative amino acid substitution(s). In certain embodiments, the amino acid substitutions do not substantially reduce the ability of the antibody to bind antigen. For example, conservative alterations (e.g., conservative substitutions as provided herein, such as in Table 3 below) that do not substantially reduce VEGFR2 binding affinity may be made. The binding affinity of anti-VEGFR2 antibody variants can be assessed using methods described in the Examples below.

Conservative substitutions are shown in Table 3 under the heading of “conservative substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, e.g., retained/improved VEGFR2 binding, decreased immunogenicity, or improved ADCC or CDC.

TABLE 3
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Non-conservative substitutions will entail exchanging a member of one of these classes for another class. An exemplary substitutional variant is an affinity matured antibody, which may be conveniently generated, e.g., using phage display based affinity maturation techniques such as those described herein. Briefly, one or more CDR residues are mutated and the variant antibodies displayed on phage and screened for a particular biological activity (e.g. binding affinity). Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).)

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, the anti-VEGFR2 antibody (or antigen binding fragment thereof) binds the same epitope of human VEGFR2 bound by a second anti-VEGFR2 antibody (or antigen binding fragment thereof). In certain embodiments, the second anti-VEGFR2 antibody (or antigen binding fragment thereof) is a variant of an anti-VEGFR2 antibody comprising a light chain variable domain sequence comprising a CDR-L1 comprising the amino acid sequence QSLYYRDGYTF (SEQ ID NO: 1); a CDR-L2 comprising the amino acid sequence LSS (SEQ ID NO: 2); and a CDR-L3 comprising the amino acid sequence MQGTHWPYT (SEQ ID NO: 3); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6), wherein the variant comprises at least one amino acid substitution in one or more of SEQ ID NOs: 1, 2, 3, 4, 5, and/or 6. In some embodiments the variant comprises at least 1, at least 2, at least 3, at least 4, at least 5, at least 6, at least 7, at least 8, at least 9, or at least 10 amino acid substitutions in one or more of SEQ ID NOs: 1, 2, 3, 4, 5, and/or 6. In certain embodiments, the amino acid substitution(s) are conservative amino acid substitution(s). In certain embodiments, the amino acid substitutions do not substantially reduce the ability of the antibody to bind antigen. For example, conservative alterations (e.g., conservative substitutions as provided herein, such as in Table 3 above) that do not substantially reduce VEGFR2 binding affinity may be made.

In certain embodiments, the anti-VEGFR2 antibody (or antigen binding fragment thereof) is a variant of an anti-VEGFR2 antibody comprising a light chain variable domain sequence comprising a CDR-L1 comprising the amino acid sequence QSLYYRDGYTF (SEQ ID NO: 1); a CDR-L2 comprising the amino acid sequence LSS (SEQ ID NO: 2); and a CDR-L3 comprising the amino acid sequence MQGTHWPYT (SEQ ID NO: 3); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6), wherein the variant comprises at least one amino acid substitution in one or more of SEQ ID NOs: 1, 2, 3, 4, 5, and/or 6. In some embodiments the variant comprises at least 1, at least 2, at least 3, at least 4, at least 5, at least 6, at least 7, at least 8, at least 9, or at least 10 amino acid substitutions in one or more of SEQ ID NOs: 1, 2, 3, 4, 5, and/or 6. In certain embodiments, the amino acid substitution(s) are conservative amino acid substitution(s). In certain embodiments, the amino acid substitutions do not substantially reduce the ability of the antibody to bind antigen. For example, conservative alterations (e.g., conservative substitutions as provided herein, such as in Table 3 above) that do not substantially reduce VEGFR2 binding affinity may be made. The binding affinity of anti-VEGFR2 antibody variants can be assessed using methods described in the Examples below.

In certain embodiments, the anti-VEGFR2 antibody competitively inhibits the binding of a second anti-VEGFR2 antibody to human VEGFR2, wherein the second anti-VEGFR2 antibody (or antigen binding fragment thereof) comprises a light chain variable domain comprising a CDR-L1 comprising the amino acid sequence QSLYYR-D/S-GYTF (SEQ ID NO: 22); a CDR-L2 comprising the amino acid sequence L/Q/R-SS (SEQ ID NO: 23); and a CDR-L3 comprising the amino acid sequence M/L/F-QGTHWPYT (SEQ ID NO: 24); and a heavy chain variable domain sequence comprising a CDR-H1 comprising the amino acid sequence G/R-F-S/T/P-FSTYA (SEQ ID NO: 25); a CDR-H2 comprising the amino acid sequence I-S/N-G-S/N-G/S-G/Q-A/T-T (SEQ ID NO: 26); and a CDR-H3 comprising the amino acid sequence KGLWFGEG-Y/L/I (SEQ ID NO: 27).

In certain embodiments, the anti-VEGFR2 antibody binds the same epitope of human VEGFR2 as a second anti-VEGFR2 antibody, wherein the second anti-VEGFR2 antibody (or antigen binding fragment thereof) comprises a light chain variable domain comprising a CDR-L1 comprising the amino acid sequence QSLYYR-D/S-GYTF (SEQ ID NO: 22); a CDR-L2 comprising the amino acid sequence L/Q/R-SS (SEQ ID NO: 23); and a CDR-L3 comprising the amino acid sequence M/L/F-QGTHWPYT (SEQ ID NO: 24); and a heavy chain variable domain sequence comprising a CDR-H1 comprising the amino acid sequence G/R-F-S/T/P-FSTYA (SEQ ID NO: 25); a CDR-H2 comprising the amino acid sequence I-S/N-G-S/N-G/S-G/Q-A/T-T (SEQ ID NO: 26); and a CDR-H3 comprising the amino acid sequence KGLWFGEG-Y/L/I (SEQ ID NO: 27).

In certain embodiments, the anti-VEGFR2 antibody (or antigen binding fragment thereof) is a variant of an anti-VEGFR2 antibody comprising a light chain variable domain comprising a CDR-L1 comprising the amino acid sequence QSLYYR-D/S-GYTF (SEQ ID NO: 22); a CDR-L2 comprising the amino acid sequence L/Q/R-SS (SEQ ID NO: 23); and a CDR-L3 comprising the amino acid sequence M/L/F-QGTHWPYT (SEQ ID NO: 24); and a heavy chain variable domain sequence comprising a CDR-H1 comprising the amino acid sequence G/R-F-S/T/P-FSTYA (SEQ ID NO: 25); a CDR-H2 comprising the amino acid sequence I-S/N-G-S/N-G/S-G/Q-A/T-T (SEQ ID NO: 26); and a CDR-H3 comprising the amino acid sequence KGLWFGEG-Y/L/I (SEQ ID NO: 27), wherein the variant comprises at least one amino acid substitution in one or more of SEQ ID NOs: 1, 2, 3, 4, 5, and/or 6. In some embodiments the variant comprises at least 1, at least 2, at least 3, at least 4, at least 5, at least 6, at least 7, at least 8, at least 9, or at least 10 amino acid substitutions in one or more of SEQ ID NOs: 1, 2, 3, 4, 5, and/or 6. In certain embodiments, the amino acid substitution(s) are conservative amino acid substitution(s). In certain embodiments, the amino acid substitutions do not substantially reduce the ability of the antibody to bind antigen. For example, conservative alterations (e.g., conservative substitutions as provided herein, such as in Table 3 above) that do not substantially reduce VEGFR2 binding affinity may be made.

In certain embodiments, the anti-VEGFR2 antibody (or antigen binding fragment thereof) comprises a light chain variable domain comprising a CDR-L1 comprising the amino acid sequence QSLYYR-D/S-GYTF (SEQ ID NO: 22); a CDR-L2 comprising the amino acid sequence L/Q/R-SS (SEQ ID NO: 23); and a CDR-L3 comprising the amino acid sequence M/L/F-QGTHWPYT (SEQ ID NO: 24); and a heavy chain variable domain sequence comprising a CDR-H1 comprising the amino acid sequence G/R-F-S/T/P-FSTYA (SEQ ID NO: 25); a CDR-H2 comprising the amino acid sequence I-S/N-G-S/N-G/S-G/Q-A/T-T (SEQ ID NO: 26); and a CDR-H3 comprising the amino acid sequence KGLWFGEG-Y/L/I (SEQ ID NO: 27).

In certain embodiments, the anti-VEGFR2 antibody or antigen binding fragment thereof comprises a light chain variable domain sequence comprising a CDR-L1 comprising an amino acid sequence selected from consisting of SEQ ID NOs: 1 and 16; a CDR-L2 comprising an amino acid sequence selected from consisting of SEQ ID NOs: 2, 7, and 8; and a CDR-L3 comprising an amino acid sequence selected from consisting of SEQ ID NOs: 3, 9, and 12; and a heavy chain variable domain sequence comprising a CDR-H1 comprising an amino acid sequence selected from consisting of SEQ ID NOs: 4 and 13-15; a CDR-H2 comprising an amino acid sequence selected from consisting of SEQ ID NOs: 5 and 17-21, and a CDR-H3 comprising an amino acid sequence selected from consisting of SEQ ID NOs: 6, and 10-11. The sequences of the CDRs noted herein are provided in Table 4 below.

TABLE 4

| SEQ ID NO: | QSLYYRDGYTF |
|---|---|
| 1 | |
| SEQ ID NO: | LSS |
| 2 | |
| SEQ ID NO: | MQGTHWPYT |
| 3 | |
| SEQ ID NO: | GFSFSTYA |
| 4 | |
| SEQ ID NO: | ISGSGGAT |
| 5 | |
| SEQ ID NO: | KGLWFGEGY |
| 6 | |
| SEQ ID NO: | QSS |
| 7 | |
| SEQ ID NO: 8 | RSS |
| SEQ ID NO: 9 | LQGTHWPYT |
| SEQ ID NO: | KGLWFGEGL |
| 10 | |
| SEQ ID NO: | KGLWFGEGI |
| 11 | |
| SEQ ID NO: | FQGTHWPYT |
| 12 | |
| SEQ ID NO: | GFTFSTYA |
| 13 | |
| SEQ ID NO: | GFPFSTYA |
| 14 | |
| SEQ ID NO: | RFSFSTYA |
| 15 | |
| SEQ ID NO: | QSLYYRSGYTF |
| 16 | |
| SEQ ID NO: | INGSGGAT |
| 17 | |
| SEQ ID NO: | ISGSSGAT |
| 18 | |
| SEQ ID NO: | ISGNGGAT |
| 19 | |
| SEQ ID NO: | ISGSGQAT |
| 20 | |
| SEQ ID NO: | ISGSGGTT |
| 21 | |

In certain embodiments, the anti-VEGFR2 antibody (or antigen binding fragment thereof) comprises a light chain variable domain sequence comprising a CDR-L1 comprising the amino acid sequence QSLYYRDGYTF (SEQ ID NO: 1); a CDR-L2 comprising the amino acid sequence LSS (SEQ ID NO: 2); and a CDR-L3 comprising the amino acid sequence MQGTHWPYT (SEQ ID NO: 3); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence MQGTHWPYT (SEQ ID NO: 3); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO:1); a CDR-L2 comprising the amino acid sequence RSS (SEQ ID NO: 8); and a CDR-L3 comprising the amino acid sequence LQGTHWPYT (SEQ ID NO: 9); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGI (SEQ ID NO:11).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence MQGTHWPYT (SEQ ID NO: 3); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence SLYYRDGYTF (SEQ ID NO: 1); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence MQGTHWPYT (SEQ ID NO: 3); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGAT (SEQ ID NO: 5); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFTFSTYA (SEQ ID NO: 13); a CDR-H2 comprising the amino acid sequence INGSGGAT (SEQ ID NO: 17); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFTFSTYA (SEQ ID NO: 13); a CDR-H2 comprising the amino acid sequence INGSGGAT (SEQ ID NO: 17); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFSFSTYA (SEQ ID NO: 4); a CDR-H2 comprising the amino acid sequence ISGSSGAT (SEQ ID NO: 18); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFSFSTYA (SEQ ID NO: 4); a CDR-H2 comprising the amino acid sequence ISGSSGAT (SEQ ID NO: 18); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence MQGTHWPYT (SEQ ID NO: 3); and a heavy chain variable domain sequence comprising a CDR-H1 comprising the amino acid sequence GFPFSTYA (SEQ ID NO: 14); a CDR-H2 comprising the amino acid sequence ISGNGGAT (SEQ ID NO: 19); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence MQGTHWPYT (SEQ ID NO: 3); and a heavy chain variable domain sequence comprising a CDR-H1 comprising the amino acid sequence RFSFSTYA (SEQ ID NO: 15); a CDR-H2 comprising the amino acid sequence ISGSGQAT (SEQ ID NO: 20); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGTT (SEQ ID NO: 21); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGTT (SEQ ID NO: 21); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFTFSTYA (SEQ ID NO: 13); a CDR-H2 comprising the amino acid sequence INGSGGAT (SEQ ID NO: 17); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFTFSTYA (SEQ ID NO: 13); a CDR-H2 comprising the amino acid sequence INGSGGAT (SEQ ID NO: 17); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFSFSTYA (SEQ ID NO: 4); a CDR-H2 comprising the amino acid sequence ISGSSGAT (SEQ ID NO: 18); and a CDR-H3 comprising the amino acid sequence KGLWFGEGY (SEQ ID NO: 6).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence LQGTHWPYT (SEQ ID NO: 9); and a heavy chain variable domain sequence comprising a CDR-H1 comprising the amino acid sequence GFSFSTYA (SEQ ID NO: 4); a CDR-H2 comprising the amino acid sequence ISGSSGAT (SEQ ID NO: 18); and a CDR-H3 comprising the amino acid sequence KGLWFGEGL (SEQ ID NO: 10).

In certain embodiments, the anti-VEGFR2 antibody or antigen binding fragment thereof comprises a light chain variable domain sequence comprising a CDR-L1 comprising the amino acid sequence QSLYYRDGYTF (SEQ ID NO: 1); a CDR-L2 comprising the amino acid sequence QSS (SEQ ID NO: 7); and a CDR-L3 comprising the amino acid sequence MQGTHWPYT (SEQ ID NO: 3); and a heavy chain variable domain sequence comprising a CDR-H1 comprising the amino acid sequence GFPFSTYA (SEQ ID NO: 14); a CDR-H2 comprising the amino acid sequence ISGNGGAT (SEQ ID NO: 19); and a CDR-H3 comprising the amino acid sequence KGLWFGEGY (SEQ ID NO: 6).

In certain embodiments, the anti-VEGFR2 antibody comprises a light chain variable domain (V_{L}) comprising an amino acid sequence set forth in any one of SEQ ID NOs: 107-113. In certain embodiments, the anti-VEGFR2 antibody comprises a heavy chain variable domain (V_{H}) comprising an amino acid sequence set forth in SEQ ID NO: 114-124. The amino acid sequences of SEQ ID NOs: 107-124 are provided below.

(SEQ ID NO: 107)
DVVMTQSPLS LPVTLGQPAS ISCRSSQSLY YRDGYTFLDWY VQKPGQSPQL
LIYLSSKRDS GVPDRISGSG SGTDFTLRIS RVEAEDVGVY YCMQGTHWPYT
FGQGTKLEIK RTVAAPSVFI FPPSDEQLKS GTASVVCLLN NFYPREAKVQ
WKVDNALQSGN
(SEQ ID NO: 108)
DVVMTQSPLS LPVTLGQPAS ISCRSSQSLY YRDGYTFLDWY VQKPGQSPQL
LIYQSSKRDS GVPDRISGSG SGTDFTLRIS RVEAEDVGVY YCMQGTHWPYT
FGQGTKLEIK RTVAAPSVFI FPPSDEQLKS GTASVVCLLN NFYPREAKVQ
WKVDNALQSGN
(SEQ ID NO: 109)
DVVMTQSPLS LPVTLGQPAS ISCRSSQSLY YRDGYTFLDWY VQKPGQSPQL
LIYRSSKRDS GVPDRISGSG SGTDFTLRIS RVEAEDVGVY YCLQGTHWPYT
FGQGTKLEIK RTVAAPSVFI FPPSDEQLKS GTASVVCLLN NFYPREAKVQ
WKVDNALQSGN
(SEQ ID NO: 110)
DVVMTQSPLS LPVTLGQPAS ISCRSSQSLY YRDGYTFLDWY VQKPGQSPQL
LIYQSSKRDS GVPDRISGSG SGTDFTLRIS RVEAEDVGVY YCLQGTHWPYT
FGQGTKLEIK RTVAAPSVFI FPPSDEQLKS GTASVVCLLN NFYPREAKVQ
WKVDNALQSGN
(SEQ ID NO: 111)
DVVMTQSPLS LPVTLGQPAS ISCRSSQSLY YRDGYTFLDWY VQKPGQSPQL
LIYQSSKRDS GVPDRISGSG SGTDFTLRIS RVEAEDVGVY YCFQGTHWPYT
FGQGTKLEIK RTVAAPSVFI FPPSDEQLKS GTASVVCLLN NFYPREAKVQ
WKVDNALQSGN
(SEQ ID NO: 112)
DVVMTQSPLS LPVTLGQPAS ISCRSSQSLY YRSGYTFLDWY VQKPGQSPQL
LIYQSSKRDS GVPDRISGSG SGTDFTLRIS RVEAEDVGVY YCMQGTHWPYT
FGQGTKLEIK RTVAAPSVFI FPPSDEQLKS GTASVVCLLN NFYPREAKVQ
WKVDNALQSGN
(SEQ ID NO: 113)
DVVMTQSPLS LPVTLGQPAS ISCRSSQSLY YRSGYTFLDWY VQKPGQSPQL
LIYQSSKRDS GVPDRISGSG SGTDFTLRIS RVEAEDVGVY YCFQGTHWPYT
FGQGTKLEIK RTVAAPSVFI FPPSDEQLKS GTASVVCLLN NFYPREAKVQ
WKVDNALQSGN
(SEQ ID NO: 114)
QVQLVQSGGG LVQPGGSLRL SCAASGFSFS TYAMSWVRQA PGKGLEWVSG
ISGSGGATHY ADSVKGRFTI SRDNSKNTVN LQMNSLRAED TAVYYCAKGL
WFGEGYWGQG TLVTVSSAST KGPSVFPLAP SSKSTSGGTA ALGCLVKDYF PEPVT
(SEQ ID NO: 115)
QVQLVQSGGG LVQPGGSLRL SCAASGFSFS TYAMSWVRQA PGKGLEWVSG
ISGSGGATHY ADSVKGRFTI SRDNSKNTVN LQMNSLRAED TAVYYCAKGL
WFGEGLWGQG TLVTVSSAST KGPSVFPLAP SSKSTSGGTA ALGCLVKDYF PEPVT
(SEQ ID NO: 116)
QVQLVQSGGG LVQPGGSLRL SCAASGFSFS TYAMSWVRQA PGKGLEWVSG
ISGSGGATHY ADSVKGRFTI SRDNSKNTVN LQMNSLRAED TAVYYCAKGL
WFGEGIWGQG TLVTVSSAST KGPSVFPLAP SSKSTSGGTA ALGCLVKDYF PEPVT
(SEQ ID NO: 117)
QVQLVQSGGG LVQPGGSLRL SCAASGFTFS TYAMSWVRQA PGKGLEWVSG
INGSGGATHY ADSVKGRFTI SRDNSKNTVN LQMNSLRAED TAVYYCAKGL
WFGEGYWGQG TLVTVSSAST KGPSVFPLAP SSKSTSGGTA ALGCLVKDYF PEPVT
(SEQ ID NO: 118)
QVQLVQSGGG LVQPGGSLRL SCAASGFTFS TYAMSWVRQA PGKGLEWVSG
INGSGGATHY ADSVKGRFTI SRDNSKNTVN LQMNSLRAED TAVYYCAKGL
WFGEGLWGQG TLVTVSSAST KGPSVFPLAP SSKSTSGGTA ALGCLVKDYF PEPVT
(SEQ ID NO: 119)
QVQLVQSGGG LVQPGGSLRL SCAASGFSFS TYAMSWVRQA PGKGLEWVSG
ISGSSGATHY ADSVKGRFTI SRDNSKNTVN LQMNSLRAED TAVYYCAKGL
WFGEGYWGQG TLVTVSSAST KGPSVFPLAP SSKSTSGGTA ALGCLVKDYF PEPVT
(SEQ ID NO: 120)
QVQLVQSGGG LVQPGGSLRL SCAASGFSFS TYAMSWVRQA PGKGLEWVSG
ISGSSGATHY ADSVKGRFTI SRDNSKNTVN LQMNSLRAED TAVYYCAKGL
WFGEGLWGQG TLVTVSSAST KGPSVFPLAP SSKSTSGGTA ALGCLVKDYF PEPVT
(SEQ ID NO: 121)
QVQLVQSGGG LVQPGGSLRL SCAASGFPFS TYAMSWVRQA PGKGLEWVSG
ISGNGGATHY ADSVKGRFTI SRDNSKNTVN LQMNSLRAED TAVYYCAKGL
WFGEGYWGQG TLVTVSSAST KGPSVFPLAP SSKSTSGGTA ALGCLVKDYF PEPVT
(SEQ ID NO: 122)
QVQLVQSGGG LVQPGGSLRL SCAASRFSFS TYAMSWVRQA PGKGLEWVSG
ISGSGQATHY ADSVKGRFTI SRDNSKNTVN LQMNSLRAED TAVYYCAKGL
WFGEGYWGQG TLVTVSSAST KGPSVFPLAP SSKSTSGGTA ALGCLVKDYF PEPVT
(SEQ ID NO: 123)
QVQLVQSGGG LVQPGGSLRL SCAASGFSFS TYAMSWVRQA PGKGLEWVSG
ISGSGGTTHY ADSVKGRFTI SRDNSKNTVN LQMNSLRAED TAVYYCAKGL
WFGEGYWGQG TLVTVSSAST KGPSVFPLAP SSKSTSGGTA ALGCLVKDYF PEPVT
(SEQ ID NO: 124)
QVQLVQSGGG LVQPGGSLRL SCAASGFSFS TYAMSWVRQA PGKGLEWVSG
ISGSGGTTHY ADSVKGRFTI SRDNSKNTVN LQMNSLRAED TAVYYCAKGL
WFGEGLWGQG TLVTVSSAST KGPSVFPLAP SSKSTSGGTA ALGCLVKDYF PEPVT

In certain embodiments, the anti-VEGFR2 antibody or antigen binding fragment thereof comprises a light chain variable domain comprising the amino acid sequence set forth in SEQ ID NO: 107 and a heavy chain variable domain comprising the amino acid sequence set forth in SEQ ID NO: 114. In certain embodiments, the anti-VEGFR2 antibody or antigen binding fragment thereof comprises a light chain variable domain comprising the amino acid sequence set forth in SEQ ID NO: 108 and a heavy chain variable domain comprising the amino acid sequence set forth in SEQ ID NO: 114. In certain embodiments, the anti-VEGFR2 antibody or antigen binding fragment thereof comprises a light chain variable domain comprising the amino acid sequence set forth in SEQ ID NO: 109 and a heavy chain variable domain comprising the amino acid sequence set forth in SEQ ID NO: 115. In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 116. In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 115. In certain embodiments, the anti-VEGFR2 antibody or antigen binding fragment thereof comprises a light chain variable domain comprising the amino acid sequence set forth in SEQ ID NO: 111 and a heavy chain variable domain comprising the amino acid sequence set forth in SEQ ID NO: 114.

In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 114. In certain embodiments, the anti-VEGFR2 antibody or antigen binding fragment thereof comprises a light chain variable domain comprising the amino acid sequence set forth in SEQ ID NO: 111 and a heavy chain variable domain comprising the amino acid sequence set forth in SEQ ID NO: 114. In certain embodiments, the anti-VEGFR2 antibody or antigen binding fragment thereof comprises a light chain variable domain comprising the amino acid sequence set forth in SEQ ID NO: 108 and a heavy chain variable domain comprising the amino acid sequence set forth in SEQ ID NO: 115. In certain embodiments, the anti-VEGFR2 antibody or antigen binding fragment thereof comprises a light chain variable domain comprising the amino acid sequence set forth in SEQ ID NO: 111 and a heavy chain variable domain comprising the amino acid sequence set forth in SEQ ID NO: 115.

In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 117. In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 118. In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 119. In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 120. In certain embodiments, the anti-VEGFR2 antibody or antigen binding fragment thereof comprises a light chain variable domain comprising the amino acid sequence set forth in SEQ ID NO: 108 and a heavy chain variable domain comprising the amino acid sequence set forth in SEQ ID NO: 121. In certain embodiments, the anti-VEGFR2 antibody or antigen binding fragment thereof comprises a light chain variable domain comprising the amino acid sequence set forth in SEQ ID NO: 112 and a heavy chain variable domain comprising the amino acid sequence set forth in SEQ ID NO: 122. In certain embodiments, the anti-VEGFR2 antibody or antigen binding fragment thereof comprises a light chain variable domain comprising the amino acid sequence set forth in SEQ ID NO: 112 and a heavy chain variable domain comprising the amino acid sequence set forth in SEQ ID NO: 123. In certain embodiments, the anti-VEGFR2 antibody or antigen binding fragment thereof comprises a light chain variable domain comprising the amino acid sequence set forth in SEQ ID NO: 112 and a heavy chain variable domain comprising the amino acid sequence set forth in SEQ ID NO: 124.

In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 117. In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 118.

In certain embodiments, the anti-VEGFR2 antibody comprises a light chain variable domain comprising the amino acid sequence set forth in SEQ ID NO: 110 and a heavy chain variable domain comprising the amino acid sequence set forth in SEQ ID NO: 119. In certain embodiments, the anti-VEGFR2 antibody or antigen binding fragment thereof comprises a light chain variable domain comprising the amino acid sequence set forth in SEQ ID NO: 110 and a heavy chain variable domain comprising the amino acid sequence set forth in SEQ ID NO: 120. In certain embodiments, the anti-VEGFR2 antibody or antigen binding fragment thereof comprises a light chain variable domain comprising the amino acid sequence set forth in SEQ ID NO: 108 and a heavy chain variable domain comprising the amino acid sequence set forth in SEQ ID NO: 121.

The heavy and light chain variable domains are combined in all possible pair-wise combinations to generate a number of anti-VEGFR2 antibodies.

In certain embodiments, the antibody comprises an Fc sequence of a human IgG, e.g., human IgG1 or human IgG4. In certain embodiments, the Fc sequence has been altered or otherwise changed so that it that lacks antibody dependent cellular cytotoxicity (ADCC) effector function, often related to their binding to Fc receptors (FcRs). There are many examples of changes or mutations to Fc sequences that can alter effector function. For example, WO 00/42072 and Shields et al. J Biol. Chem. 9(2): 6591-6604 (2001) describes antibody variants with improved or diminished binding to FcRs. The contents of those publications are specifically incorporated herein by reference. The antibody can be in the form of a Fab, Fab′, a F(ab)′2, single-chain Fv (scFv), an Fv fragment; a diabody and a linear antibody. Also, the antibody can be a multi-specific antibody that binds to EGFR, but also binds one or more other targets and inhibits their function. The antibody can be conjugated to a therapeutic agent (e.g., cytotoxic agent, a radioisotope and a chemotherapeutic agent) or a label for detecting VEGFR2 in patient samples or in vivo by imaging (e.g., radioisotope, fluorescent dye and enzyme).

Nucleic acid molecules encoding the anti-VEGFR2 antibodies, expression vectors comprising nucleic acid molecules encoding the CDRs and/or a heavy chain variable domain and/or a light chain variable domain described herein, and cells comprising the nucleic acid molecules are also contemplated. These antibodies can be used in the therapies described herein and to detect VEGFR2 protein in patient samples (e.g., via FACS, immunohistochemistry (IHC), ELISA assays) or in patients.

Functional Characteristics

In certain embodiments, an anti-VEGFR2 antibody provided herein has a stronger binding affinity for a VEGFR2 than it has for a homologue of VEGFR2, such as VEGFR1/FLT1 or VEGFR3/FLT4/PCL/Chy. Normally, an anti-VEGFR2 antibody provided herein “binds specifically” to VEGFR2 (i.e., has a binding affinity (Kd) value of no more than about 1×10⁻⁷ M, preferably no more than about 1×10⁻⁸ and most preferably no more than about 1×10⁻⁹ M) but has a binding affinity for a member of the VEGFR family which is at least about 50 fold, or at least about 500 fold, or at least about 1000 fold, weaker than its binding affinity for VEGFR2. In certain embodiments, an anti-VEGFR2 antibody provided herein has a Kd of less than 1×10⁻⁹ M. The anti-VEGFR2 antibody that binds specifically to VEGFR2 can be of any of the various types of antibodies as defined above, but preferably is a humanized or human antibody.

In some embodiments, the extent of binding of the anti-VEGFR2 antibody to a non-target protein (such as VEGFR1/FLT1 or VEGFR3/FLT4/PCL/Chy) is less than about 10% of the binding of the antibody to VEGFR2 as determined by methods known in the art, such as ELISA, fluorescence activated cell sorting (FACS) analysis, or radioimmunoprecipitation (RIA). Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope.

In certain embodiments, the anti-VEGFR2 antibody also binds to VEGFR3.

In certain embodiments, the anti-VEGFR2 antibody binds a human VEGFR2 with a Kd between about 0.1 pM to 200 pM (0.2 nM), e.g., about 0.1 pM, about 0.25 pM, about 0.5 pM, about 0.75 pM, about 1 pM, about 5 pM, about 10p M, about 20 pM, about 30 pM, about 40 pM, about 50 pM, about 60 pM, about 70 pM, about 80 pM, about 90 pM, about 100 pM, about 110 pM, about 120 pM, about 130 pM, about 140 pM, about 150 pM, about 160 pM, about 170 pM, about 180 pM, about 190 pM, or more than about 190 pM, including any range between these values. In certain embodiments the binding affinity of the anti-VEGFR2 antibody to VEGFR2 is about 1%, about 5%, about 10%, about 15%, about 20%, about 30%, about 40%, about 50%, about 60%, about 70%, about 80%, about 90%, about 95% about 96%, about 97%, about 98%, about 99%, about 100%, or more than about 100% higher (e.g., about 105%, about 110%, about 120%, or about 130%) higher than the binding affinity of 1121B (see US 2009/0306348) or 1121N (see U.S. Pat. No. 7,498,414) to VEGFR2. In certain embodiments, the binding affinity of the anti-VEGFR2 to VEGFR2 is about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.25-fold, about 2.5-fold, about 2.75 fold, about 3-fold, about 3.25-fold, about 3.5 fold, about 3.75-fold, about 4-fold, about 4.25-fold, about 4.5-fold, about 4.75-fold, or more than about 4.75 fold higher than the binding affinity of 1121B (see US 2009/0306348) or 1121N (see U.S. Pat. No. 7,498,414) to VEGFR2, including any range in between these values.

In certain embodiments, the anti-VEGFR2 antibody binds human umbilical vein endothelial cells (HUVECS). In certain embodiments, the anti-VEGFR2 antibody inhibits HUVEC tube formation. In certain embodiments, the anti-VEGFR2 antibody inhibits HUVEC migration. In certain embodiments, the anti-VEGFR2 antibody inhibits HUVEC survival. In certain embodiments, the anti-VEGFR2 antibody inhibits HUVEC proliferation. In certain embodiments, the anti-VEGFR2 antibody inhibits HUVEC sprouting.

In certain embodiments, the anti-VEGFR2 antibody inhibits angiogenesis in vitro. In certain embodiments, the anti-VEGFR2 antibody inhibits angiogenesis in vivo.

In certain embodiments, the anti-VEGFR2 antibody inhibits VEGF-C stimulated human lymphatic endothelial cell (HLEC) proliferation. In certain embodiments, the anti-VEGFR2 antibody inhibits VEGF-C stimulated VEGFR-2 phosphorylation. In certain embodiments, the anti-VEGFR2 antibody also inhibits VEGF-C stimulated VEGFR-3 phosphorylation.

### Monoclonal Antibodies

Monoclonal antibodies can be prepared, e.g., using hybridoma methods, such as those described by Kohler and Milstein, Nature, 256:495 (1975) or can be made by recombinant DNA methods (U.S. Pat. No. 4,816,567) or can be produced by the methods described herein in the Examples below. In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro.

The immunizing agent will typically include a polypeptide or a fusion protein of the protein of interest or a composition comprising the protein. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, MONOCLONAL ANTIBODIES: PRINCIPLES AND PRACTICE (New York: Academic Press, 1986), pp. 59-103. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high-level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al. MONOCLONAL ANTIBODY PRODUCTION TECHNIQUES AND APPLICATIONS (Marcel Dekker, Inc.: New York, 1987) pp. 51-63.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub clones can be isolated or purified from the culture medium or ascites fluid by conventional immunoglobulin purification procedures such as, for example, protein A-Sepharose, hydroxyapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies provided herein can be readily isolated and sequenced using conventional procedures (e.g., by using oligonucleotide probes that are capable of binding specifically to genes encoding the heavy and light chains of murine antibodies). The hybridoma cells provided herein serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains in place of the homologous murine sequences (U.S. Pat. No. 4,816,567; Morrison et al., supra) or by covalently joining to the immunoglobulin coding sequence all or part of the coding sequence for a nonimmunoglobulin polypeptide. Such a non-immunoglobulin polypeptide can be substituted for the constant domains of an antibody provided herein, or can be substituted for the variable domains of one antigen-combining site of an antibody provided herein to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using, but not limited to, techniques known in the art.

### Human and Humanized Antibodies

The antibodies can be humanized antibodies or human antibodies. Humanized forms of non-human (e.g., murine) antibodies are chimeric immunoglobulins, immunoglobulin chains, or fragments thereof (such as Fv, Fab, Fab′, F(ab′)₂, or other antigen-binding subsequences of antibodies) that typically contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody preferably also will comprise at least a portion of an immunoglobulin constant region (Fc), typically that of a human immunoglobulin. Jones et al. Nature, 321: 522-525 (1986); Riechmann et al., Nature, 332: 323-329 (1988); Presta, Curr. Op. Struct. Biol., 2:593-596 (1992).

Generally, a humanized antibody has one or more amino acid residues introduced into it from a source that is non-human. These non-human amino acid residues are often referred to as “import” residues, which are typically taken from an “import” variable domain. According to one embodiment, humanization can be essentially performed following the method of Winter and co-workers (Jones et al. Nature, 321: 522-525 (1986); Riechmann et al. Nature, 332: 323-327 (1988); Verhoeyen et al. Science, 239: 1534-1536 (1988)), by substituting rodent CDRs or CDR sequences for the corresponding sequences of a human antibody. Accordingly, such “humanized” antibodies are antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al. PNAS USA, 90:2551 (1993); Jakobovits et al., Nature, 362:255-258 (1993); Bruggemann et al. Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al. Nature Biotechnology, 14: 845-851 (1996); Neuberger, Nature Biotechnology, 14: 826 (1996); Lonberg and Huszar, Intern. Rev. Immunol., 13: 65-93 (1995).

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to one embodiment of this technique, antibody V domain sequences are cloned in frame into either a major or minor coat protein gene of a filamentous bacteriophage, such as M13 or fd, and displayed as functional antibody fragments on the surface of the phage particle. Phage display can be performed in a variety of formats, e.g., as described below in the Examples section or as reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Human antibodies can also be produced using various techniques known in the art, including phage display libraries. Hoogenboom and Winter, J. Mol. Biol., 227: 381 (1991); Marks et al., J. Mol. Biol., 222: 581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Multi-Specific Antibodies

Multi-specific antibodies are monoclonal, preferably human or humanized, antibodies that have binding specificities for two or more different antigens (e.g., bispecific antibodies have binding specificities for at least two antigens). For example, one of the binding specificities can be for the a5-1 protein, the other one can be for any other antigen. According to one preferred embodiment, the other antigen is a cell-surface protein or receptor or receptor subunit. For example, the cell-surface protein can be a natural killer (NK) cell receptor. Thus, according to one embodiment, a bispecific antibody of this invention can bind both VEGFR2 and, e.g., a second cell surface receptor.

Suitable methods for making bispecific antibodies are well known in the art. For example, the recombinant production of bispecific antibodies is based on the co-expression of two immunoglobulin heavy-chain/light-chain pairs, where the two heavy chains have different specificities. Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829 and in Traunecker et al., EMBO, 10: 3655-3659 (1991).

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH 1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology, 121: 210 (1986).

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., PNAS USA, 90:6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147:60 (1991).

### Heteroconjugate Antibodies

Heteroconjugate antibodies are composed of two covalently joined antibodies. Such antibodies have, for example, been proposed to target immune-system cells to unwanted cells (U.S. Pat. No. 4,676,980), and for treatment of HIV infection. WO 91/00360; WO 92/200373; EP 03089. It is contemplated that the antibodies can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

### Effector Function Engineering

It can be desirable to modify the antibody provided herein with respect to effector function, so as to enhance, e.g., the effectiveness of the antibody in treating a pathological condition associated with excessive angiogenesis, such as cancer (e.g., colon cancer, colorectal cancer, gastric cancer, gastroesophageal cancer, bladder cancer, lung cancer, and solid tumor) or an on ocular disease (e.g., those described elsewhere herein). For example, cysteine residue(s) can be introduced into the Fc region, thereby allowing inter-chain disulfide bond formation in this region. The homodimeric antibody thus generated can have improved internalization capability and/or increased complement-mediated cell killing and antibody-dependent cellular cytotoxicity (ADCC). See, Caron et al., J. Exp. Med., 176: 1191-1195 (1992) and Shapes, J. Immunol., 148:2918-2922 (1992). Homodimeric antibodies with enhanced anti-tumor activity can also be prepared using heterobifunctional cross-linkers as described in Wolff et al., Cancer Research, 53: 2560-2565 (1993). Alternatively, an antibody can be engineered that has dual Fc regions and can thereby have enhanced complement lysis and ADCC capabilities. See, Stevenson et al., Anti-Cancer Drug Design: 219-230 (1989).

Mutations or alterations in the Fc region sequences can be made to improve FcR binding (e.g., FcγR, FcRn). According to one embodiment, an antibody of this invention has at least one altered effector function selected from the group consisting of ADCC, CDC, and improved FcRn binding compared to a native IgG or a parent antibody. Examples of several useful specific mutations are described in, e.g., Shields, R L et al. (2001) JBC 276(6)6591-6604; Presta, L. G., (2002) Biochemical Society Transactions 30(4):487-490; and WO 00/42072.

According to one embodiment, the Fc receptor mutation is a substitution at least one position selected from the group consisting of: 238, 239, 246, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 332, 333, 334, 335, 337, 338, 340, 360, 373, 376, 378, 382, 388, 389, 398, 414, 416, 419, 430, 434, 435, 437, 438 or 439 of the Fc region, wherein the numbering of the residues in the Fc region is according to the EU numbering system. In some embodiments, the Fc receptor mutation is a D265A substitution. In some embodiments, the Fc receptor mutation is a N297 A substitution. Additional suitable mutations are set forth in U.S. Pat. No. 7,332,581.

In certain embodiments, an anti-VEGFR2 antibody provided herein is afucosylated (i.e., an “afucosylated anti-VEGFR2 antibody” or a “non-fucosylated anti-VEGFR2 antibody”). “Afucosylated antibody” or “nonfucosylated antibody” refers to an antibody of IgG1 or IgG3 isotype with an altered pattern of glycosylation in the Fc region at Asn297 having a reduced level of fucose residues. Glycosylation of human IgG1 or IgG3 occurs at Asn297 as core fucosylated bianntennary complex oligosaccharide glycosylation terminated with up to 2 Gal residues. These structures are designated as G0, G1. (a1,6 or a1,3) or G2 glycan residues, depending from the amount of terminal Gal residues (Raju, T. S., BioProcess Int. 1 (2003) 44-53). CHO type glycosylation of antibody Fe parts is e.g. described by Routier, Glycoconjugate J. 14 (1997) 201-207. Antibodies which are recombinantly expressed in non glycomodified CHO host cells usually are fucosylated at Asn297 in an amount of at least 85%. In certain embodiments, an afucosylated anti-VEGFR2 antibody provided herein has a reduced level of fucose residues. In certain embodiments, an afucosylated anti-VEGFR2 antibody provided herein has no fucose in its glycosylation pattern. It is commonly known that typical glycosylated residue position in an antibody is the asparagine at position 297 according to the EU numbering system (“Asn297”).

Thus, in certain embodiments an afucosylated anti-VEGFR2 antibody provided herein comprises an Fc sequence has been altered or otherwise changed so that it a reduced level of fucose residues or no fucose in its glycosylation pattern. In certain embodiments, an anti-VEGFR2 antibody provided herein comprises an Fc sequence having an alteration at position 297 according to the EU numbering system.

In certain embodiments, an afucosylated anti-VEGFR2 antibody provided herein is produced by a host cell capable of producing hypo- or afucosylated glycans. Stable mammalian host cell lines that can produce afucosylated antibodies have been established and are described in, e.g., Yamane-Ohnuki et al. (2004) Biotechnol Bioeng. 87, 614-622; Mori et al. (2004) Biotechnol Bioeng. 88, 901-908; Kanda et al (2006) Biotechnol Bioeng. 94, 680-688; Kanda (2007) J Biotechnol. 130, 300-310; Imai-Nishiya (2007) BMC Biotechnol 7, 84; Yamane-Ohnuki and Satoh (2009) mAbs 1, 230-236. In certain embodiments, an afucosylated anti-VEGFR2 antibody provided herein is expressed in a glycomodified host cell engineered to express b(1,4)-N-acetylglucosaminyltransferase III activity. In certain embodiments, an afucosylated anti-VEGFR2 antibody provided herein is expressed in a glycomodified host cell in which 1,6-fucosyltransferase activity has been decreased or eliminated. See, e.g., U.S. Pat. No. 6,946,292 for details regarding the production of glycomodified host cells. The amount of antibody fucosylation can be predetermined e.g. either by fermentation conditions (e.g. fermentation time) or by combination of at least two antibodies with different fucosylation amount. Such afucosylated antibodies and respective glycoengineering methods are described in WO 2005/044859, WO 2004/065540, WO 2007/031875, Umana et al., Nature Biotechnol. 17 (1999) 176-180, WO 991154342, WO 2005/018572, WO 2006/116260, WO 2006/114700, WO 2005/011735, WO 2005/027966, WO 97/1028267, US 2006/0134709, US 2005/0054048, US 2005/0152894, WO 2003/035835, WO 2000/061739. These glycoengineered antibodies have an increased ADCC. Other glycoengineering methods yielding afucosylated antibodies according to the invention are described e.g. in Niwa, R. et al., J. Immunol. Methods 306 (2005) 151-160; Shinkawa, T., et al., J. Biol. Chem, 278 (2003) 3466-3473; WO 03/055993 or US 2005/0249722.

In certain embodiments, an afucosylated anti-VEGFR2 antibody provided herein is produced using in vitro techniques. In certain embodiments, an afucosylated anti-VEGFR2 antibody provided herein is chemically synthesized. (See, e.g., Yamamoto et al. (2008) JAGS 130, 501-510, which describes the chemical synthesis of a non-fucosylated form of monocyte chemotactic protein 3 (MCP-3). In certain embodiments, an afucosylated anti-VEGFR2 antibody provided herein is produced by using a fucosidase to remove fucose residues on IgGs. (See, e.g., Yazawa et al. (1986) Biochem Biophys Res Commun. 136, 563-569.

In certain embodiments, an afucosylated anti-VEGFR2 antibody provided herein has improved antibody-dependent cell-mediated cytotoxicity (ADCC) effector function compared to a fucosylated anti-VEGFR2 antibody, as demonstrated by assays well known to those of ordinary skill in the art. See, e.g., Suzuki et al. (2007) Clin Cancer Res 13, 1875. For example, in certain embodiments, ADCC effector function activity of an afucosylated anti-VEGFR2 antibody described herein is at least about 140%, at least about 150%, at least about 160%, at least about 170%, at least about 180%, at least about 190%, at least about 190%, at least about 200%, at least about 210%, at least about 220%, at least about 230%, at least about 240%, at least about 250%, at least about 260%, at least about 270%, at least about 280%, at least about 290%, or at least about 300% of the ADCC effector function activity a fucosylated anti-VEGFR2 antibody, including any range between these values. In certain embodiments, ADCC effector function activity of an anti-VEGFR2 antibody described herein is more about 300% of the ADCC effector function activity of a fucosylated anti-VEGFR2 antibody, including at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, at least about 510%, at least about 520%, at least about 530%, at least about 540%, at least about 550%, at least about 560%, at least about 570%, at least about 580%, at least about 590%, or at least about 600% of the ADCC effector function activity of a fucosylated anti-VEGFR2 antibody, including any range between these values.

### Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re, and ¹⁸⁶Re. Exemplary chemotherapeutic agents useful in the generation of such immunoconjugates include those described elsewhere herein.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bisdiazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, WO94/11026.

In another embodiment, the antibody can be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

### Covalent Modifications

Covalent modifications of the anti-VEGFR2 antibodies and fragments thereof are included within the scope of this invention. One type of covalent modification includes reacting targeted amino acid residues of a polypeptide with an organic derivatizing agent that is capable of reacting with selected side chains or the N- or C-terminal residues of the polypeptide. Derivatization with bifunctional agents is useful, for instance, for crosslinking the polypeptide to a water-insoluble support matrix or surface for use in the method for purifying antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane and agents such as methyl-3-[(p-azidophenyl)-dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Other modifications include the conjugation of toxins to the antagonists such as maytansine and maytansinoids, calicheamicin and other cytotoxic agents.

Another type of covalent modification of the polypeptide comprises linking the polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

### Chimeric Molecules

An anti-VEGFR2 antibody (or fragment thereof) of the present invention can also be modified if advantageous in a way to form a chimeric molecule comprising the polypeptide fused to another, heterologous polypeptide or amino acid sequence (e.g., immunoadhesins or peptibodies).

In one embodiment, such a chimeric molecule comprises a fusion of the polypeptide with a protein transduction domain which targets the polypeptide for delivery to various tissues and more particularly across the brain blood barrier, using, for example, the protein transduction domain of human immunodeficiency virus TAT protein (Schwarze et al., 1999, Science 285: 1569-72).

In another embodiment, such a chimeric molecule comprises a fusion of the polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag. Various tag polypeptides and their respective antibodies are known in the art. Examples include poly-histidine (poly-His) or poly-histidine-glycine (poly-His-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule can comprise a fusion of the polypeptide with an immunoglobulin or a particular region of an immunoglobulin. For a bivalent form of the chimeric molecule (e.g., an “immunoadhesin”), such a fusion could be to the Fc region of an IgG molecule. Ig fusions of this invention include polypeptides that comprise approximately or only residues 94-243, residues 33-53 or residues 33-52 of human in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also, U.S. Pat. No. 5,428,130 issued Jun. 27, 1995.

### Immunoliposomes

The antibodies disclosed herein can also be formulated as immunoliposomes. Liposomes containing the antibody are prepared by methods known in the art, such as described in Epstein et al., PNAS USA, 82: 3688 (1985); Hwang et al., PNAS USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. An anti-neoplastic agent, a growth inhibitory agent, or a chemotherapeutic agent (such as doxorubicin) is optionally also contained within the liposome. See, Gabizon et al., J. National Cancer Inst., 81(19): 1484 (1989).

### Treatment Using Anti-Vascular Endothelial Growth Factor Receptor 2 (VEGFR2) Antibodies

In another aspect of the invention, anti-VEGFR2 antibodies are used to inhibit angiogenesis. VEGFR stimulation of vascular endothelium is associated with angiogenic diseases and vascularization of tumors. Accordingly, the human anti-VEGFR2 antibodies provided herein are effective for treating subjects with vascularized tumors or neoplasms or angiogenic diseases. Such tumors and neoplasms include, for example, malignant tumors and neoplasms, such as blastomas, carcinomas or sarcomas, and highly vascular tumors and neoplasms. Cancers that may be treated by the methods provided by the present invention include, for example, cancers of the brain, genitourinary tract, lymphatic system, stomach, kidney (renal cancer), colon, larynx and lung and bone. Non-limiting examples further include epidermoid tumors, squamous tumors, such as head and neck tumors, peritoneal cancer, colorectal tumors, colorectal cancer, rectal tumors, rectal cancer, prostate tumors, breast tumors, persistent, recurrent, or metastatic carcinoma of the cervix, lung tumors, including lung adenocarcinoma and small cell and non-small cell lung tumors, pancreatic tumors, thyroid tumors, fallopian tube tumors, ovarian tumors (such as, recurrent epithelial ovarian cancer) and liver tumors. The method is also used for treatment of vascularized skin cancers, including squamous cell carcinoma, basal cell carcinoma, and skin cancers that can be treated by suppressing the growth of malignant keratinocytes, such as human malignant keratinocytes. Other cancers that can be treated include Kaposi's sarcoma, CNS neoplasms (neuroblastomas, capillary hemangioblastomas, meningiomas and cerebral metastases), melanoma, gastrointestinal and renal cell carcinomas and sarcomas, rhabdomyosarcoma, glioblastoma, including glioblastoma multiforme, and leiomyosarcoma.

A further aspect of the present invention includes methods of treating or preventing pathologic conditions characterized by excessive angiogenesis, involving, for example, vascularization and/or inflammation, such as atherosclerosis, rheumatoid arthritis (RA), neovascular glaucoma, proliferative retinopathy including proliferative diabetic retinopathy, macular degeneration, hemangiomas, angiofibromas, and psoriasis. Other non-limiting examples of non-neoplastic angiogenic disease are retinopathy of prematurity (retrolental fibroplasia), corneal graft rejection, insulin-dependent diabetes mellitus, multiple sclerosis, myasthenia gravis, Crohn's disease, autoimmune nephritis, primary biliary cirrhosis, acute pancreatitis, allograft rejection, allergic inflammation, contact dermatitis and delayed hypersensitivity reactions, inflammatory bowel disease, septic shock, osteoporosis, osteoarthritis, cognition defects induced by neuronal inflammation, Osler-Weber syndrome, restinosis, and fungal, parasitic and viral infections, including cytomegaloviral infections. In certain embodiments, the pathologic condition characterized by excessive angiogenesis is an ocular disease. In certain embodiments, the ocular disease is selected from the group consisting of retinopathy, age-induced macular degeneration (e.g., wet AMD), diabetic macular edema, rubeosis, uveitis resulting from psoriasis, an inflammatory renal disease, haemolytic uremic syndrome, diabetic nephropathy (e.g., proliferative diabetic retinopathy), inflammatory bowel disease, chronic inflammation, chronic retinal detachment, chronic uveitis, chronic vitritis, corneal graft rejection, corneal neovascularization, corneal graft neovascularization, myopia, ocular neovascular disease, Pagets disease, pemphigoid, polyarteritis, post-laser radial keratotomy, retinal neovascularization, Sogrens syndrome, ulcerative colitis, graft rejection, lung inflammation, nephrotic syndrome, edema, and neovascular glaucoma. In certain embodiments, the invention provides anti-VEGFR2 antibodies (or fragments thereof) for use in the manufacture of a medicament for the treatment of an ocular disease such as, e.g., those described above). In certain embodiments, the invention provides anti-VEGFR2 antibodies (or fragments thereof) for use in treating ocular disease (such as those described above) in a subject. In certain embodiments, the subject to be treated is a mammal (e.g., human, non-human primate, rat, mouse, cow, horse, pig, sheep, goat, dog, cat, etc.). In certain embodiments, the subject is a human. In certain embodiments, the subject is a clinical patient, a clinical trial volunteer, an experimental animal, etc. In certain embodiments, the subject is suspected of having or at risk for having an ocular disease (such as those described above). In certain embodiments, the subject has been diagnosed with an ocular disease (such as those described above).

The anti-VEGFR2 antibodies (or fragments thereof) and/or compositions provided herein can be administered to subjects (e.g., mammals such as humans) to treat diseases and disorders involving abnormal VEGFR2 activity, including, for example, cancer (such as, e.g., colon cancer, colorectal cancer, gastric cancer, gastroesophageal cancer, bladder cancer, lung cancer, and solid tumor) or pathological conditions characterized by excessive angiogenesis (such as those described above). In certain embodiments, the invention provides anti-VEGFR2 antibodies (or fragments thereof) for use in the manufacture of a medicament for the treatment of cancer (such as, e.g., colon cancer, colorectal cancer, gastric cancer, gastroesophageal cancer, bladder cancer, lung cancer, and solid tumor) or a pathological condition characterized by excessive angiogenesis (such as those described above) in a subject. In certain embodiments, the invention provides anti-VEGFR2 antibodies (or fragments thereof) for use in treating cancer (such as, e.g., colon cancer, colorectal cancer, gastric cancer, gastroesophageal cancer, bladder cancer, lung cancer, and solid tumor) or a pathological condition characterized by excessive angiogenesis (such as those described above) in a subject. In certain embodiments, the subject to be treated is a mammal (e.g., human, non-human primate, rat, mouse, cow, horse, pig, sheep, goat, dog, cat, etc.). In certain embodiments, the subject is a human. In certain embodiments, the subject is a clinical patient, a clinical trial volunteer, an experimental animal, etc. In certain embodiments, the subject is suspected of having or at risk for having a cancer (such as, e.g., colon cancer, colorectal cancer, gastric cancer, gastroesophageal cancer, bladder cancer, lung cancer, and solid tumor) or a pathological condition characterized by excessive angiogenesis (such as those described above). In certain embodiments, the subject has been diagnosed with a cancer (such as, e.g., colon cancer, colorectal cancer, gastric cancer, gastroesophageal cancer, bladder cancer, lung cancer, and solid tumor) or a pathological condition characterized by excessive angiogenesis (such as those described above).

Many diagnostic methods for cancer (such as, e.g., colon cancer, colorectal cancer, gastric cancer, gastroesophageal cancer, bladder cancer, lung cancer, and solid tumor) or a pathological condition characterized by excessive angiogenesis (such as those described above) and the clinical delineation of those diseases are known in the art. Such methods include, but are not limited to, e.g., immunohistochemistry, PCR, fluorescent in situ hybridization (FISH). Additional details regarding diagnostic methods for abnormal VEGFR2 activity or expression are described in, e.g., Gupta et al. (2009) Mod Pathol. 22(1): 128-133; Lopez-Rios et al. (2013) J Clin Pathol. 66(5): 381-385; Ellison et al. (2013) J Clin Pathol 66(2): 79-89; and Guha et al. (2013) PLoS ONE 8(6): e67782. A clinician skilled in the art can readily determine, for example, by the use of clinical tests, physical examination and medical/family history, if an individual is a candidate for treatment with an anti-VEGFR2 antibody described herein.

Administration can be by any suitable route including, e.g., intravenous, intramuscular, or subcutaneous. In some embodiments, the anti-VEGFR2 antibodies (or fragments thereof) and/or compositions provided herein are administered in combination with a second, third, or fourth agent (including, e.g., an antineoplastic agent, a growth inhibitory agent, a cytotoxic agent, or a chemotherapeutic agent) to treat the diseases or disorders involving abnormal VEGFR2 activity. Such agents include, e.g., docetaxel, gefitinib, FOLFIRI (irinotecan, 5-fluorouracil, and leucovorin), irinotecan, cisplatin, carboplatin, paclitaxel, bevacizumab (anti-VEGF antibody), FOLFOX-4 (infusional fluorouracil, leucovorin, and oxaliplatin, afatinib, gemcitabine, capecitabine, pemetrexed, tivantinib, everolimus, CpG-ODN, rapamycin, lenalidomide, vemurafenib, endostatin, lapatinib, PX-866, Imprime PGG, and erlotinib. In some embodiments, the anti-VEGFR2 antibodies (or fragments thereof) are conjugated to the additional agent.

Depending on the indication to be treated and factors relevant to the dosing that a physician of skill in the field would be familiar with, the antibodies provided herein will be administered at a dosage that is efficacious for the treatment of that indication while minimizing toxicity and side effects. For the treatment of a cancer (such as, e.g., colon cancer, colorectal cancer, gastric cancer, gastroesophageal cancer, bladder cancer, lung cancer, and solid tumor) or a pathological condition characterized by excessive angiogenesis (such as described elsewhere herein), a typical dose can be, for example, in the rage of 0.001 to 1000 μg; however, doses below or above this exemplary range are within the scope of the invention. The daily dose can be about 0.1 μg/kg to about 100 mg/kg of total body weight (e.g., about 5 μg/kg, about 10 μg/kg, about 100 μg/kg, about 500 μg/kg, about 1 mg/kg, about 50 mg/kg, or a range defined by any two of the foregoing values), preferably from about 0.3 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 1 mg/kg of total body weight (e.g., about 3 μg/kg, about 15 μg/kg, about 75 μg/kg, about 300 μg/kg, about 900 μg/kg, or a range defined by any two of the foregoing values), and even more preferably from about 0.5 to 10 mg/kg body weight per day (e.g., about 2 mg/kg, about 4 mg/kg, about 7 mg/kg, about 9 mg/kg, or a range defined by any two of the foregoing values). As noted above, therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

A pharmaceutical composition comprising the anti-VEGFR2 antibody can be administered one, two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection

The antibody (or antigen-binding fragment thereof) may be administered in a single daily dose, or the total daily dose may be administered in divided dosages of two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection or surgically implanted in various locations.

Cancer treatments can be evaluated by, e.g., but not limited to, tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In some embodiments, the efficacy of treatment is measured as the percentage tumor growth inhibition (% TGI), calculated using the equation 100-(T/C×100), where T is the mean relative tumor volume of the treated tumor, and C is the mean relative tumor volume of a non-treated tumor. In certain embodiments, the % TGI is about 10%, about 20%, about 30%, about 40%, about 50%, about 60%, about 70%, about 80%, about 90%, about 91%, about 92%, about 93%, about 94%, about 95%, or more than 95%, including any range in between these values. In certain embodiments the % TGI of an anti-VEGFR2 is the same as or greater than the % TGI of 1121B (see US 2009/0306348) or 1121N (see U.S. Pat. No. 7,498,414), such as about 0.7-fold, 0.8-fold, 0.9-fold, 1.0-fold 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, or more than about 2.7-fold greater than the % TGI of 1121B (see US 2009/0306348) or 1121N (see U.S. Pat. No. 7,498,414).

### Pharmaceutical Formulations

The anti-VEGFR2 antibodies (or fragments thereof) can be formulated with suitable carriers or excipients so that they are suitable for administration. Suitable formulations of the antibodies are obtained by mixing an antibody (or fragment thereof) having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary antibody formulations are described in WO98/56418, expressly incorporated herein by reference. Lyophilized formulations adapted for subcutaneous administration are described in W097/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent. Such molecules are suitably present in combination in amounts that are effective for the purpose intended. The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages. The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Sustained-release preparations may be prepared. Suitable examples of sustained release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and. ethyl-L-glutamate, non-degradable ethylene-vinyl, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

Lipofectins or liposomes can be used to deliver the polypeptides and antibodies (or fragments thereof) or compositions of this invention into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., PNAS USA, 90: 7889-7893 (1993).

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's PHARMACEUTICAL SCIENCES, supra.

Sustained-release preparations can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydro gels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In certain embodiments, the formulation comprises an anti-VEGFR2 antibody described herein at a concentration of greater than about 0.5 mg/ml, greater than about 1 mg/ml (such as about 1.1, about 1.2, about 1.3, about 1.4, about 1.5, about 1.6, about 1.7, about 1.8, about 1.9, or greater than about 1.9 mg/ml), greater than about 2 mg/ml (such as about 2.1, about 2.2, about 2.3, about 2.4, about 2.5, about 2.6, about 2.7, about 2.8, about 2.9, or greater than about 2.9 mg/ml), greater than about 3 mg/ml, greater than about 4 mg/ml, greater than about 5 mg/ml, greater than about 6 mg/ml, greater than about 7 mg/ml, greater than about 8 mg/ml, greater than about 9 mg/ml, greater than about 10 mg/ml, greater than about 11 mg/ml, greater than about 12 mg/ml, greater than about 13 mg/ml, greater than about 14 mg/ml, or about 15 mg/ml, including any range in between these values.

In certain embodiments, the anti-VEGFR2 antibody is formulated in a buffer comprising a citrate, histidine, acetate, phosphate, sucrose, NaCl, succinate, glycine, polysorbate 80 (Tween 80), or any combination of the foregoing. In certain embodiments, the anti-VEGFR2 antibody is formulated in a buffer comprising about 10 mM citrate, about 10 mM acetate, or about 10 mM phosphate. In certain embodiments, the anti-VEGFR2 antibody is formulated in a buffer comprising about 50 mM to about 100 mM NaCl. In certain embodiments, the anti-VEGFR2 antibody is formulated in a buffer comprising about 0.5% to about 3% glycine. In certain embodiments, the anti-VEGFR2 antibody is formulated in a buffer comprising about 0.005% to about 0.02% polysorbate 80 (Tween 80). In certain embodiments, the anti-VEGFR2 antibody is formulated in a buffer having a pH between about 5.5 and 6.5. In certain embodiments, the anti-VEGFR2 antibody is formulated in a buffer comprising 10 mM citrate, 75 mM NaCl, 2% glycine, 2% sucrose and 0.02% polysorbate 80, wherein the formulation is at pH=6.0. In certain embodiments, the anti-VEGFR2 antibody is formulated in a buffer comprising 10 mM acetate, 75 mM NaCl, 2% glycine, 2% sucrose and 0.02% polysorbate 80, wherein the formulation is at pH=6.0. In certain embodiments, the anti-VEGFR2 antibody is formulated in a buffer comprising 10 mM phosphate, 75 mM NaCl, 2% glycine, 2% sucrose and 0.02% polysorbate 80, wherein the formulation is at pH=6.0.

In certain embodiments, a formulation comprising an anti-VEGFR2 antibody provided herein is stable at −80° C. for about 0.5 weeks, 1.0 weeks, 1.5 weeks, 2.0 weeks, 2.5 weeks, 3.5 weeks, 4.0 weeks, 4.5 weeks, or 5.0 weeks, including any range in between these values. In certain embodiments, a formulation comprising an anti-VEGFR2 antibody provided herein (such as a formulation comprising 10 mM citrate, 75 mM NaCl, 2% glycine, 2% sucrose and 0.02% polysorbate 80, wherein the formulation is at pH=6.0) is stable under accelerated conditions (such as storage at about 40° C.) for about 0.5 weeks, 1.0 weeks, 1.5 weeks, 2.0 weeks, 2.5 weeks, 3.5 weeks, 4.0 weeks, 4.5 weeks, or 5.0 weeks, including any range in between these values.

The formulations to be used for in vivo administration must be sterile. This is readily accomplished by, e.g., filtration through sterile filtration membranes.

### Methods of Diagnosis and Imaging Using Anti-Epidermal Growth Factor Receptor Antibodies

Labeled anti-VEGFR2 antibodies, fragments thereof, and derivatives and analogs thereof, which specifically bind to a VEGFR2 polypeptide can be used for diagnostic purposes to detect, diagnose, or monitor diseases and/or disorders associated with the expression, aberrant expression and/or activity of VEGFR2. For example, the anti-VEGFR2 antibodies (or fragments thereof) provided herein can be used in in situ, in vivo, ex vivo, and in vitro diagnostic assays or imaging assays. Methods for detecting expression of a VEGFR2 polypeptide, comprising (a) assaying the expression of the polypeptide in cells (e.g., tissue) or body fluid of an individual using one or more antibodies of this invention and (b) comparing the level of gene expression with a standard gene expression level, whereby an increase or decrease in the assayed gene expression level compared to the standard expression level is indicative of aberrant expression.

Additional embodiments provided herein include methods of diagnosing a disease or disorder associated with expression or aberrant expression of VEGFR2 in an animal (e.g., a mammal such as a human). The methods comprise detecting VEGFR2 molecules in the mammal. In certain embodiments, diagnosis comprises: (a) administering an effective amount of a labeled anti-VEGFR2 antibody (or fragment thereof) to a mammal (b) waiting for a time interval following the administering for permitting the labeled anti-VEGFR2 antibody (or fragment thereof) to preferentially concentrate at sites in the subject where the VEGFR2 molecule is expressed (and for unbound labeled molecule to be cleared to background level); (c) determining background level; and (d) detecting the labeled molecule in the subject, such that detection of labeled molecule above the background level indicates that the subject has a particular disease or disorder associated with expression or aberrant expression of EGFR. Background level can be determined by various methods including, comparing the amount of labeled molecule detected to a standard value previously determined for a particular system.

Anti-VEGFR2 antibodies (or fragments thereof) provided herein can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹³¹I, ¹²⁵I, ¹²³I, ¹²¹I), carbon (¹⁴C), sulfur ³⁵S), tritium (³H), indium (^{115m}In, ^{113m}In, ¹¹²In, ¹¹¹In), and technetium (⁹⁹Tc, ^{99m}Tc), thallium ²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru; luminol; and fluorescent labels, such as fluorescein and rhodamine, and biotin.

Techniques known in the art may be applied to labeled antibodies (or fragments thereof) provided herein. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003). Alternatively, or additionally, one can measure levels of a VEGFR2 polypeptide-encoding nucleic acid or mRNA in the cell, e.g., via fluorescent in situ hybridization using a nucleic acid based probe corresponding to an EGFR-encoding nucleic acid or the complement thereof; (FISH; see W098/454 79 published October, 1998), Southern blotting, Northern blotting, or polymerase chain reaction (PCR) techniques, such as real time quantitative PCR (RT-PCR). One can also study VEGFR2 overexpression by measuring shed antigen in a biological fluid such as serum, e.g., using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al., J. Immunol. Methods 132:73-80 (1990)). Aside from the above assays, various in vivo and ex vivo assays are available to the skilled practitioner. For example, one can expose cells within the body of the mammal to an antibody which is optionally labeled with a detectable label, e.g., a radioactive isotope, and binding of the antibody to the can be evaluated, e.g., by external scanning for radioactivity or by analyzing a sample (e.g., a biopsy or other biological sample) taken from a mammal previously exposed to the antibody.

### Articles of Manufacture and Kits

Another embodiment of the invention is an article of manufacture containing materials useful for the treatment of cancer (such as, e.g., colon cancer, colorectal cancer, gastric cancer, gastroesophageal cancer, bladder cancer, lung cancer, and solid tumor) or a pathological condition characterized by excessive angiogenesis (such as those described elsewhere herein). The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating the condition and may have a sterile access port (for example the container may be an intravenous solution bag or a vial having a stopper pierceable by a hypodermic injection needle). At least one active agent in the composition is an anti-VEGFR2 antibody (or fragment thereof) provided herein. The label or package insert indicates that the composition is used for treating the particular condition. The label or package insert will further comprise instructions for administering the antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In one embodiment, the package insert indicates that the composition is used for treating cancer (such as, e.g., colon cancer, colorectal cancer, gastric cancer, gastroesophageal cancer, bladder cancer, lung cancer, and solid tumor) or a pathological condition characterized by excessive angiogenesis (such as those described elsewhere herein).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for isolation or detection of VEGFR2 in patients, optionally in combination with the articles of manufacture. For isolation and purification of EGFR, the kit can contain an anti-VEGFR2 antibody (or fragment thereof) provided herein coupled to beads (e.g., sepharose beads). Kits can be provided which contain the antibodies (or fragments thereof) for detection and quantitation of VEGFR2 in vitro, e.g. in an ELISA or a Western blot. As with the article of manufacture, the kit comprises a container and a label or package insert on or associated with the container. For example, the container holds a composition comprising at least one anti-VEGFR2 antibody provided herein. Additional containers may be included that contain, e.g., diluents and buffers, control antibodies. The label or package insert may provide a description of the composition as well as instructions for the intended in vitro or diagnostic use.

### EXAMPLES

### Example 1. Generation, Affinity Maturation, and Characterization of Anti-Vascular Endothelial Growth Factor Receptor 2 (VEGFR2) Antibodies

Ten positive anti-VEGFR2 antibody clones (i.e., V1-V10) were identified by screening of a human phage display library with VEGFR2-Fc. In vitro functional assays, described in further detail below and summarized in Table 5, were performed to characterize the clones. The assays were performed using 1121B (also referred to herein as Ref-A), i.e., an anti-VEGFR2 antibody described in US 2009/0306348, and/or 1121N (also referred to herein as Ref-B), i.e., an anti-VEGFR2 antibody described in U.S. Pat. No. 7,498,414 as positive control(s).

TABLE 5

| | Ab | | | | | | | | | |
|---|---|---|---|---|---|---|---|---|---|---|
| Assay | V1 | V2 | V4 | V5 | V6 | V7 | V8 | V9 | V10 | Ref-A |
| ECD (D1-7) | ++/+++ | +++ | ++ | ++ | +++ | ++ | ++ | +++ | ++ | ++++ |
| Domains 2-3 | − | − | − | + | − | − | ++ | − | − | ++++ |
| Domains 1-3 | − | − | − | + | +++ | − | ++ | − | − | ++++ |
| Domains 1-4 | − | − | − | + | ++/+++ | − | +++ | − | − | + |
| Sequences | K | K | K | K | K | λ | K | K | K | K |
| Ligand-binding | − | − | − | − | − | − | − | − | − | +++ |
| blocking | | | | | | | | | | |
| Competition assay | − | ND | ND | ND | ND | ND | ND | − | − | +++ |
| (compete with IMC- | | | | | | | | | | |
| 1121B) | | | | | | | | | | |
| Inhibition of tube | − | +++ | − | − | ++++ | ++ | ++++ | ++ | + | +++ |
| formation (2-D | | | | | | | | | | |
| angiogenesis) (BD | | | | | | | | | | |
| HUVEC) | | | | | | | | | | |
| Inhibition of tube | − | +++ | ND | ND | ++++ | ++ | ++++ | ++ | + | +++ |
| formation (BD + BCRC | | | | | | | | | | |
| HUVEC) | | | | | | | | | | |
| Inhibition of HUVEC | ++++ | ++++ | ND | ND | − | − | + | + | + | + |
| migration (BD + BCRC | | | | | | | | | | |
| HUVEC) | | | | | | | | | | |
| Inhibition of HUVEC | ++++ | − | ND | ND | − | − | − | ++++ | ++/+++ | +++/++++ |
| survival (BD + BCRC | | | | | | | | | | |
| HUVEC) | | | | | | | | | | |
| Inhibition of HUVEC | +++/++++ | − | ND | ND | −/+ | + | − | ++++ | + | +++ |
| proliferation | | | | | | | | | | |
| (BD + BCRC HUVEC) | | | | | | | | | | |
| Sprouting assay (3-D | + | ND | ND | ND | ND | ND | ND | ++ | ND | +++ |
| angiogenesis) | | | | | | | | | | |
| Martrigel plug assay | ND | ND | ND | ND | ND | ND | ND | ++++ | ND | ++++ |
| (in vivo angiogenesis) | | | | | | | | | | |

Rows 1-4 of Table 5 provide the results of ELISA assays performed to assess the binding of V1-V10 to domains 1-7 of VEGFR2, domains 2-3 of VEGFR2, domains 1-3 of VEGFR2, and domains 1-4 of VEGFR2, respectively. As shown in row 2, V5 and V8 can bind domains 2-3 of VEGFR2; however, V5 binds with low affinity.

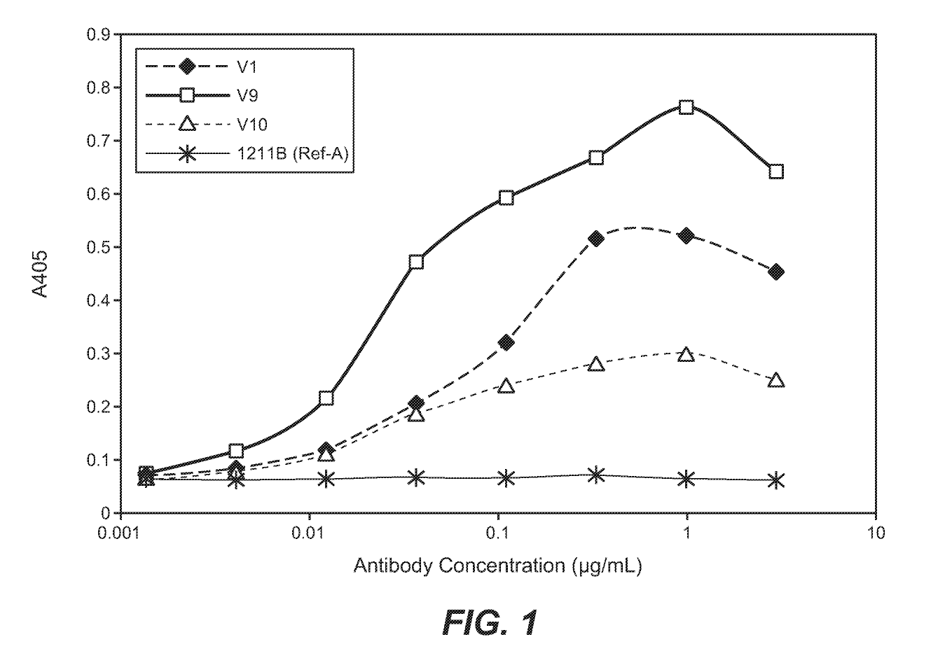

Additional ELISA assays were performed to assess the binding of V1, V9, V10, and 1121B to domains 5-7 of VEGFR2. Briefly, serial dilutions of V1, V9, V10, and 1121B captured with sheep anti-human fd antibody in wells of a microtiter dish. VEGFR2-D5-7 binding was measured by adding VEGFR2-D5-7-AP fusion protein to wells. Following an incubation and wash, pNPP was added to the wells and incubated for 30 minutes. AP activity was measured by monitoring the increase in absorbance at 405 nm. Antibody concentration was plotted as a function of AP activity (FIG. 1). As shown in FIG. 1, clones V1, V9, and V10 bind domains 5-7 of VEGFR2, whereas 1121B does not.

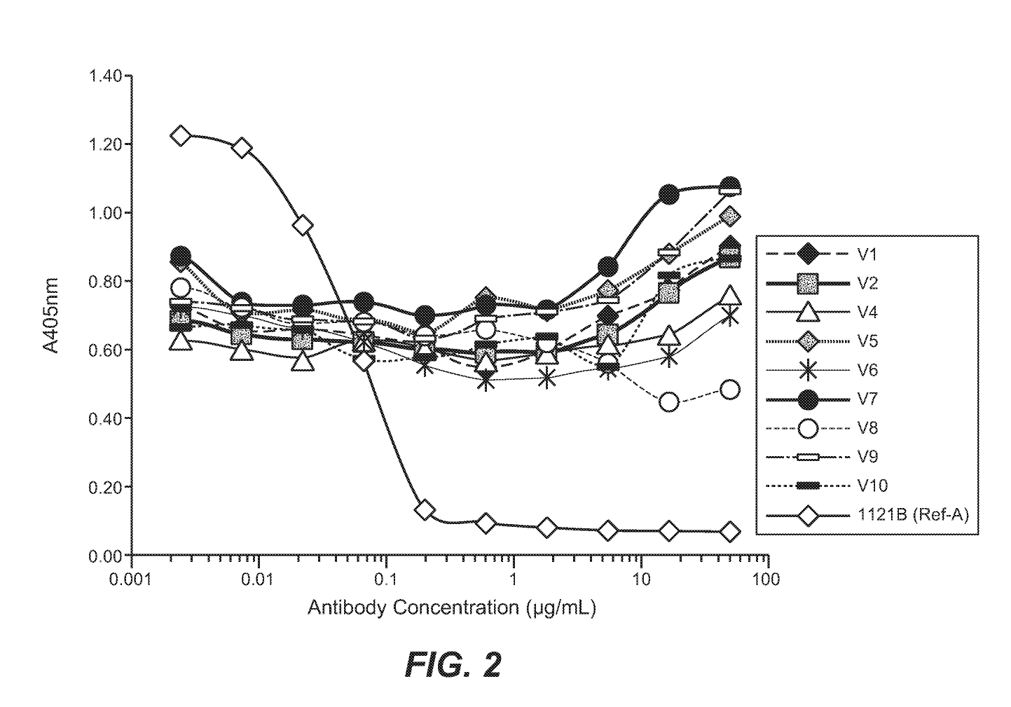

Row 6 of Table 5 shows that clones V1-V10 were not found to block the binding of VEGFR2 to VEGF. Briefly, serial dilutions of each clone were incubated with VEGFR2-AP at 28° C. for 2 hours. Each antibody:antigen mixture was added to VEGF-coated wells of a microtiter dish. Following an incubation and wash, pNPP was added to the wells and incubated for 30 minutes. AP activity was measured by monitoring the increase in absorbance at 405 nm. Antibody concentration was plotted as a function of AP activity (FIG. 2). As shown in FIG. 2, 1121B blocked the binding of VEGFR2 to VEGF, but clones V1-V10 did not. V1, V9, and V10 were also determined to not compete with 1121B for binding to VEGFR2 in a competitive ELISA. (See row 7 of Table 5.)

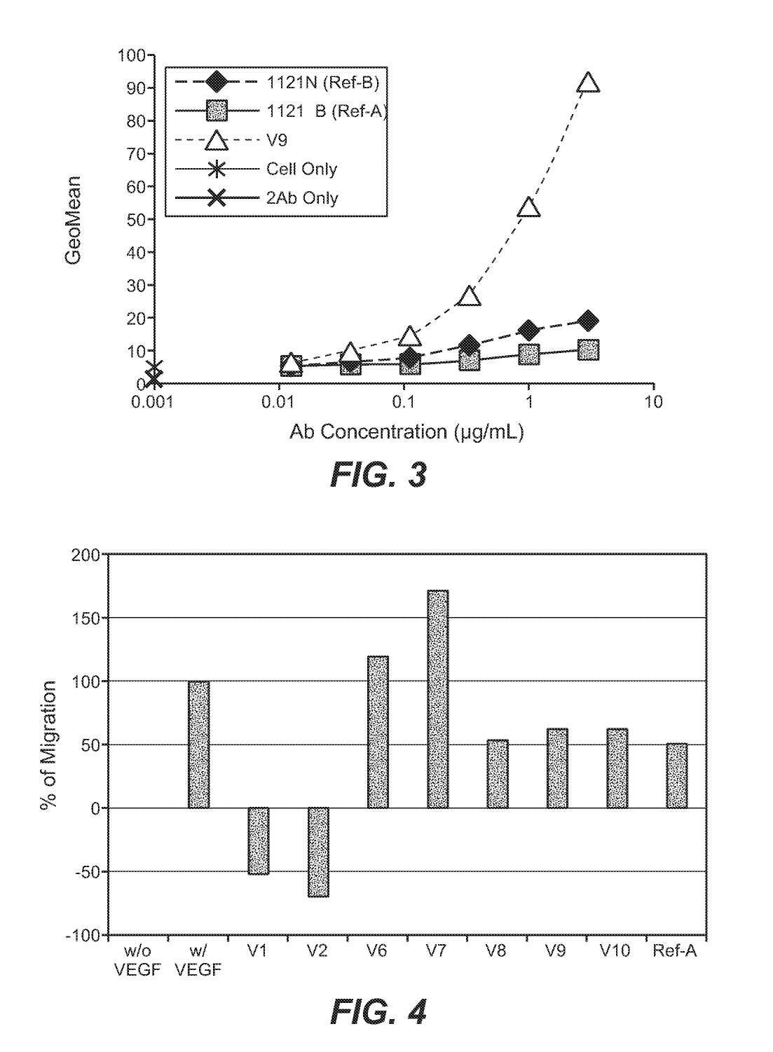

Next, the binding of clones V1-V10 to human umbilical vein endothelial cells (HUVECs, which express VEGFR2) was assayed. Briefly, HUVECs (2×10⁵) were washed once with PBS, fixed with 4% paraformaldehyde for 30 minutes at 4° C., washed with FACS buffer (1% fetal bovine serum in PBS), and transferred to the wells of a 96-well plate (2×10⁵ cells/well). Following a centrifugation at 500×g for 5 minutes at 4° C., 100 μl antibody (3-fold diluted from a 3μl/ml stock) was added to a well and incubated for 30 minutes at 4° C. The cells were then washed twice with FACS buffer and incubated with anti-Fcr-FITC antibody for 30 minutes at 4° C. The cells were then washed three times with FACS buffer, fixed with 4% paraformaldehyde for 30 minutes at 4° C., and centrifuged at 500×g for 5 minutes at 4° C. The supernatants were discarded. The cells were resuspended in FACS buffer and analyzed via flow cytometry. As shown in FIG. 3, whereas clone V9 was found to bind HUVECs, neither 1121B (Ref A) nor 1211N (Ref B) were found to bind HUVECs.

Rows 8 and 9 of Table 5 provide the qualitative results of HUVEC tube formation assays performed to assess the abilities of clones V1-V10 to inhibit angiogenesis. HUVECs form capillary-like tubular structures when cultured on a gel of growth factor-reduced basement membrane extracts. The assays were performed as follows: Thawed Matrigel (growth-factor reduced) was added to μ-Slides (IBIDI) and allowed to polymerize at 37° C. for 1 hour. Cultured HUVECs (from BD Biosciences) were detached from plates, washed 3 times, and resuspended in medium (EBM-2+10% “used” EGM-2 (i.e., cultured for 2 overnights, then collected and filtered)+5 ng/ml bFGF). 50 μl of HUVEC were incubated with 0.2 μg/ml or 5 μg/ml of each antibody for 20 minutes at 37° C. 50 ng/ml of VEGF-165 (i.e., a 165 amino acid splice variant of VEGF-A) was added to each plate, and the plates were incubated at 37° C. for 6 hours. When 5 μg/ml of each antibody was tested, V8 exhibited stronger inhibition of HUVEC tube formation than 1121B. 1121B exhibited stronger inhibition of HUVEC tube formation than V5, V6 or V9. V5, V6 and V9 exhibited stronger inhibition of HUVEC tube formation than V2 or V10. V7, V4, and V1 did not exhibit inhibition of HUVEC tube formation at 5 μg/ml. (Data not shown.) When 0.2 μg/ml of each antibody was tested, V8 and 1121B exhibited stronger inhibition of HUVEC tube formation than V2 or V6. V2 and V6 exhibited stronger inhibition of HUVEC tube formation than V10, V9, V4, and V4. V5 and V1 did not exhibit inhibition of HUVEC tube formation at 0.2 μg/ml. (Data not shown.)

HUVEC tube formation assays were repeated using a mixture of HUVECs from BD Biosciences and the Bioresource Collection and Research Center. 2500 ng/ml, 50 ng/ml, and 1 ng/ml of each antibody was tested. When 2500 ng/ml of each antibody was used, 1121B, V7, V8, V9, and V10 exhibited stronger inhibition of HUVEC tube formation than V1, V2, and V6. (Data not shown.) When 50 ng/ml of each antibody was used, 1121B and V6 exhibited stronger inhibition of HUVEC tube formation than V1, V2, V7, V8, and V9. V10 did not exhibit inhibition of HUVEC tube formation at 50 ng/ml. (Data not shown.) When 1 ng/ml of each antibody was used, 1121B and V7 exhibited stronger inhibition of HUVEC tube formation than V6 and V10. V8, V9, V1 and V2 not exhibit inhibition of HUVEC tube formation at ing/ml. (Data not shown.)

Row 10 of Table 5 provides the qualitative results of HUVEC migration assays performed to assess the abilities of clones V1-V10 to inhibit the migration activity of HUVECs. The assays were performed as follows: Confluent HUVEC (8×10⁵ cells/plate) were subcultured for 26 hours before being detached, washed three times in EBM-2 (Endothelial Basal Medium-2), and resuspended in EBM-2 at a concentration of about 5×10⁵ cells/μl. 0.1 ml of cell suspension was incubated with 30 μg/ml antibody at 37° C. for 20 minutes. 8-micron pore-sized transwell permeable support inserts (Millipore) were rinsed with 50 ml PBS and coated with 50 μl of 10 μg/mlfibronectin (2 μg/cm²) for 3 hours. The coating solution was removed, and the cell suspension/antibody mixtures were transferred to the transwells. The transwells were transferred to wells with lower compartments containing 0.7 ml EBM-2 with or without 400 ng/ml VEGF165-Fc. Following a 16-17 hour incubation, the remaining cells were removed, and the transwells were washed twice with PBS. The migrated cells were fixed with 100% methanol for 20 minutes, dried, and then stained with 0.15% crystal violet in 0.1M borate/2% ethanol pH=9 for 30 minutes. The stained transwell was then washed with dH₂O), and the upper face of the transwell was cleaned with a cotton swab. The stained cells were extracted with 60 μl of 10% acetic acid. Absorbance values were measured at 590 nM. % Migration was determined according to the formula below:

$$\% Migration = \frac{{OD}_{590} sample - {OD}_{590} without VEGF}{{OD}_{590} with VEGF - {OD}_{590} without VEGF} \times 100$$

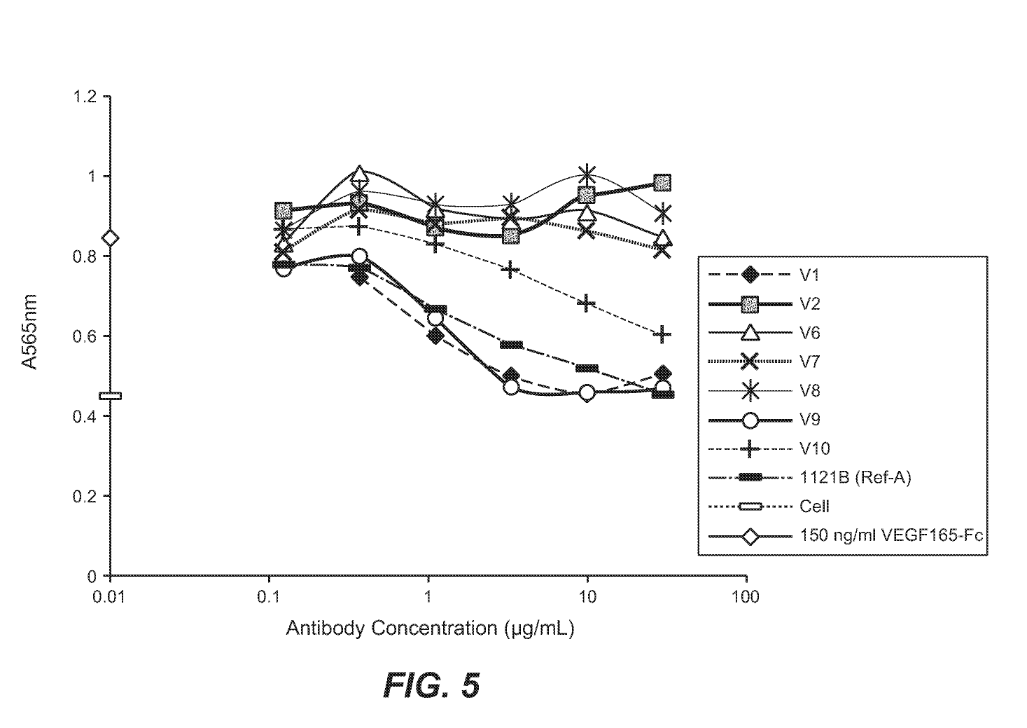

V1 and V2 demonstrated stronger inhibition of HUVEC migration than V8, V9, V10, and 1211B, See FIG. 4.

Row 11 of Table 5 provides the qualitative results of HUVEC survival assays performed to assess the abilities of clones V1-V10 to inhibit HUVEC survival. Briefly, HUVECs were grown in 48-well culture plates with 800 μl EGM-2 medium until confluence and then washed three times with PBS. Serial dilutions of anti-VEGFR2 antibodies were added to the wells and incubated at 37° C. for 30 minutes. Following the addition of VEGF-165-Fc to each well, the cells were incubated at 37° C. (5% CO₂) for 2 days. On Day 3, 80 μl of 5 mg/ml MTT (i.e., a yellow tetrazole that reduced to purple formazan in living cells) was added to each well and incubated for 3 hours. 100 of DMSO was added to each well, and the absorbance at 565 nm for each well was measured. V9, V1 and 1121B (Ref A) exhibited stronger inhibition of HUVEC survival than V10. V7, V6, V2, and V8 not exhibit inhibition of HUVEC survival. (See FIG. 5.)

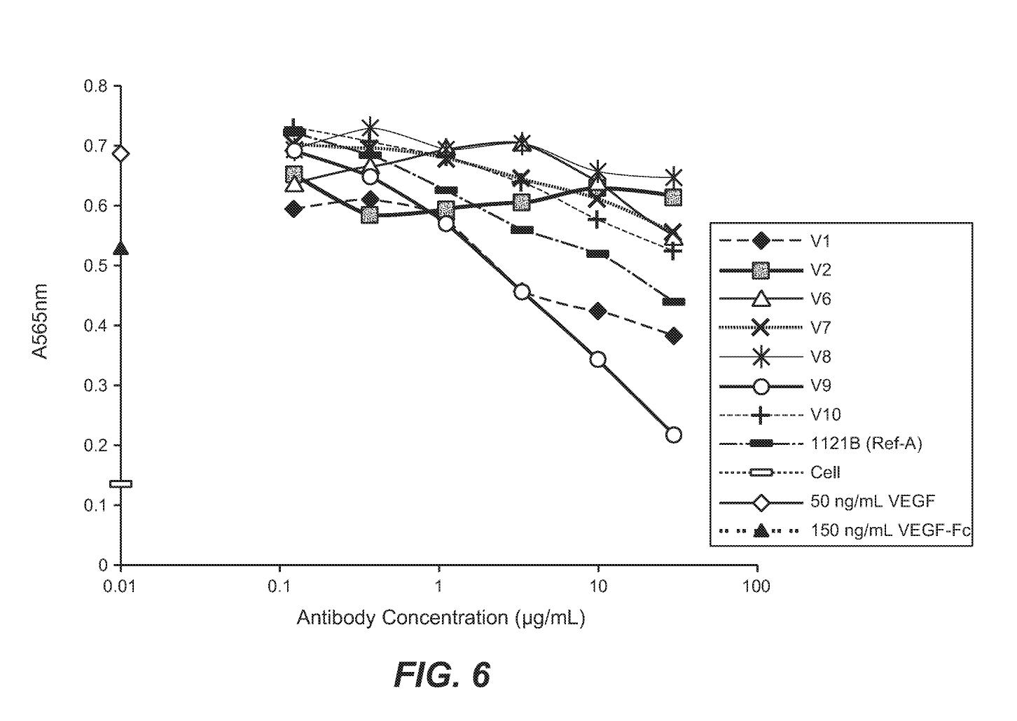

Row 12 of Table 5 provides the qualitative results of HUVEC cell proliferation assays performed to assess the abilities of clones V1-V10 to inhibit HUVEC proliferation. The assays were performed as follows: HUVECs were detached from culture plates, resuspended in EBM-2 medium, and seeded into wells of a 48-well tissue culture place. Serial dilutions of each antibody were added to the wells and incubated at 37° C. for 30 minutes. Following the addition of VEGF-165 to each well, the cells were incubated at 37° C. (5% CO₂) for 3 days. At the end of the incubation, 80 μl of 5 mg/ml MTT was added to each well and incubated for 3 hours. 100 of DMSO was added to each well and the absorbance at 565 nm for each well was measured. V9 exhibited stronger inhibition of HUVEC proliferation than V1 and 1121B. V1 and 1121B exhibited stronger inhibition of HUVEC survival than V10 and V7. V6, V2, and V8 not exhibit inhibition of HUVEC proliferation. (See FIG. 6.)

Row 13 of Table 5 provides the qualitative results of in vitro HUVEC sprouting assays performed to assess the abilities of V1-V10 to inhibit HUVEC sprouting (i.e., 3-dimensional angiogenesis) induced by angiogenic factors. Briefly, 25 ng/ml of VEGF₁₆₅ (eBioscience), 20 ng/ml of bFGF (eBioscience), and increasing concentrations of 1121B (Ref A), Avastin, V1 or V9 were added to a media:methocel (40:60) solution (100 μl). This was then mixed 1:1 with a working collagen solution (in 1×PBS, pH 7.0). Immediately after mixing, the matrix was added to a pre-warmed 48-well plate and returned to 37° C. Meanwhile, HUVEC which were induced to form spheroids of 750 cells in untreated sterile 96-well microplate for 18 h were collected (150 xg, no brake), washed once with PBS, and then suspended in EBM-2 (˜50 spheroids/mL). The suspended spheroids were transferred to microtubes and pelleted. After aspirating the supernatant, the media:methocel solution (150 μl) with growth factors and antibodies was layered over the pellet followed by the addition of 150 μl working collagen stock. The solution was immediately mixed and transferred to the plates containing the base coat of matrix. After a 2-hour incubation at 37° C., 150 μl of media containing growth factors and antibodies was added to the surface of each well. The sprouts were imaged after a 48-64 hours incubation at 37° C.

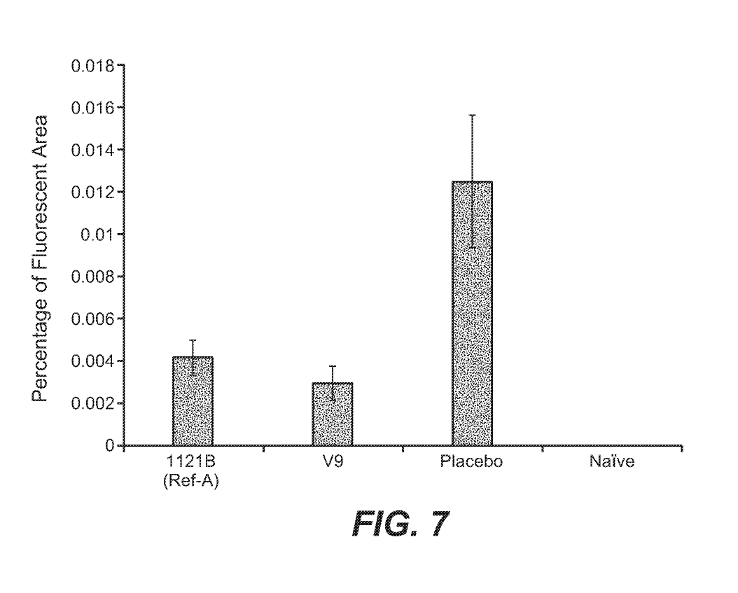

Row 14 of Table 5 provides the qualitative results of matrigel plug assays performed to assess the abilities of V1-V9 to inhibit angiogenesis in vivo. The experiment was conducted in severe combined immunodeficient mice (SCID) mice. HUVEC spheroids were prepared by pipetting 1000 HUVECs in a hanging drop on untreated 96-well microplate to allow overnight spheroid formation. The following day, HUVEC spheroids were harvested and mixed in a Matrigel solution with single HUVECs to reach a final number of 100 HUVECs as spheroids and 200,000 single HUVECs per injected plug. VEGF165 and fibroblast growth factor 2 (FGF-2) was added at a final concentration of 1000 ng/mL. Two SCID mice per treatment group were subcutaneously injected with 0.5 mL of the cell/matrix suspension that quickly polymerized. Twice weekly dosing of the antibodies started from Day 0 (4 hours after the cell/matrix inoculation). On Day 18, the study was terminated. The Matrigel plugs were removed, photographed and fixed in 4% paraformaldehyde at room temperature for 4 to 12 hours. Thereafter the plugs were paraffin embedded with a standard procedure. For histological examination of the human vasculature, paraffin sections (thickness 8-10 μm) were prepared from all plugs. Human blood vessel formation were detected by staining with mouse anti-human CD34 (Dako, Catalog#M716501) and FITC-conjugated rabbit anti-mouse secondary antibody (Dako, Catalog#F026102). Two sections per plug were analyzed and 5 images were taken from each section at a magnification of 200× using the Eclipse TE2000-U microscope (Nikon). The level of human vasculature was determined by the average percentage of FITC-positive (CD34-positive) area. As shown in FIG. 7, V9 inhibited angiogenesis in vivo to the same degree as 1211B (Ref A).

Mice bearing human HCT-116 human colon cancer tumor xenografts were used to assay the therapeutic efficacy of clones V1, V9, and V10. Briefly, human HCT-116 human colon cancer cells (inoculum=1×10⁶ cells) were implanted into female BALB/c nude mice. The mice were randomized into 7 groups. Each group was treated with one of the dosing regimens described in Table 6 below:

TABLE 6

| | | | Treatment | | Treatment |
|---|---|---|---|---|---|
| Group | Animals | Tumor | Agent (Ab) | Dose | Schedule |
| 1 | BALB/c NU | HCT-116 | 1121B | 100 mg/kg | Day 3 after |
| 2 | Female | Inoculum = | V9 | 100 mg/kg | inoculation; |
| 3 | 5 weeks old | 1 × 10⁶ cells | 1121B | 50 mg/kg | twice a week |
| 4 | 4 mice/group | | V9 | 50 mg/kg | for 5 weeks |
| 5 | | | V1 | 50 mg/kg | |
| 6 | | | V10 | 50 mg/kg | |
| 7 | | | Placebo | 10 ml/kg | |

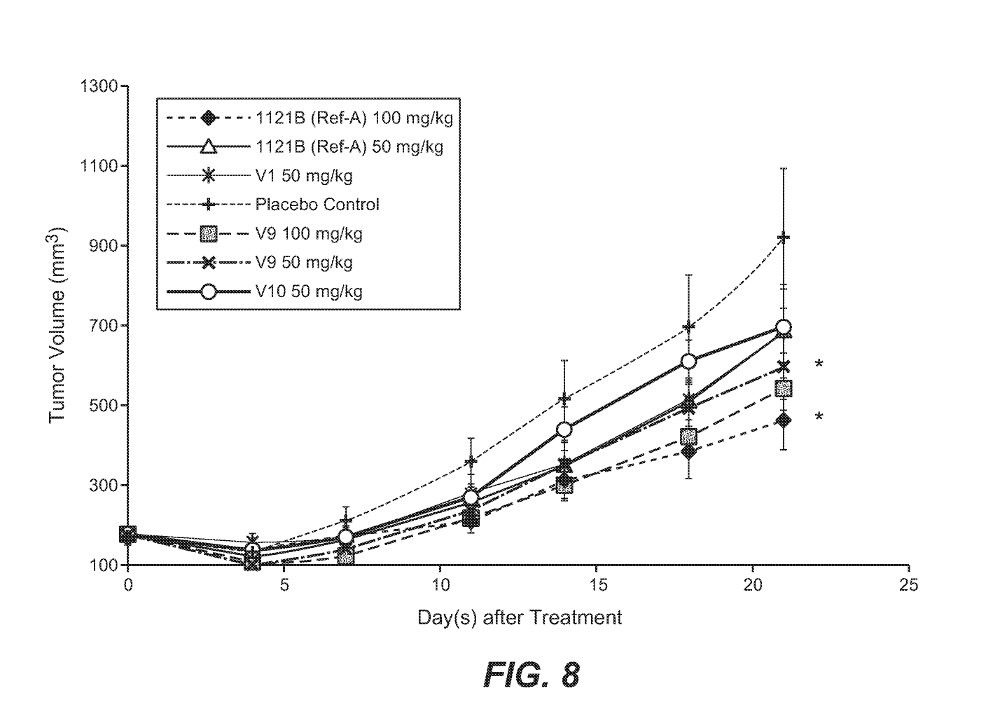

21 days following the beginning of treatment, tumors in mice treated with V9 (100 mg/kg) were about 51% smaller than tumors in mice receiving placebo, and tumors in mice treated with 1121B (100 mg/kg) were about 61% smaller than tumors in mice receiving placebo. 21 days following the beginning of treatment, tumors in mice treated with V9 (50 mg/kg) were about 43% smaller than tumors in mice receiving placebo, and tumors in mice treated with 1121B (50 mg/kg) were about 31% smaller than tumors in mice receiving placebo. See FIG. 8 and Table 7 below.

TABLE 7

| | | RTV² | TV/ | TGI %⁴ | | |
|---|---|---|---|---|---|---|
| | TV¹ | (21 | CV %³ | (21 | p Value of TV⁵ | |
| | (21 Days) | Days) | (21 Days) | Days) | (21 Days) | |
| 1121B | 462 | 304% | 50% | 61% | 0.010 | <0.05* |
| (100 mg/kg) | | | | | | |
| V9 | 542 | 317% | 59% | 51% | 0.0308 | <0.05* |
| (100 mg/kg) | | | | | | |
| 1121B | 689 | 431% | 75% | 31% | 0.121 | >0.05 |
| (50 mg/kg) | | | | | | |
| V9 | 596 | 358% | 65% | 43% | 0.060 | >0.05 |
| (50 mg/kg) | | | | | | |
| V1 | 684 | 406% | 74% | 32% | 0.146 | >0.05 |
| (50 mg/kg) | | | | | | |
| V10 | 700 | 429% | 76% | 29% | 0.148 | >0.06 |
| (50 mg/kg) | | | | | | |
| Placebo | 919 | 640% | — | — | — | — |

¹TV: Tumor volume
²RTV: Tumor volume relative to initial
³TV/CV %: Treatment group volume/Control group volume
⁴TGI %: Tumor growth inhibition rate = 1 − (T21 − T0)/(C21 − C0)%
⁵p value: <0.05 = * <0.01 = ** <0.001 = ***

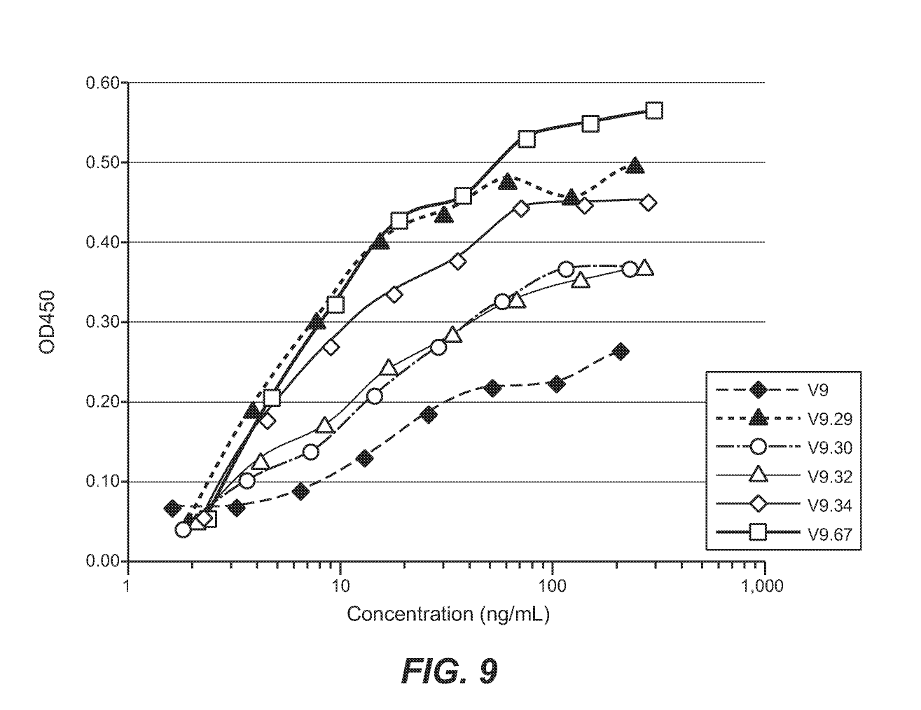

Clone V9 was then used in further in vitro phage display-based affinity maturation experiments to generate additional clones with improved binding performance. First, CDR-L2/CDR-L3/CDR-H3 nucleic acid libraries were generated via PCR, cloned into a phage display vector, and transformed into E. coli to produce a library of phages. After 3 rounds of panning, 77 Fab clones were screened via ELISA, and five clones (i.e., 29, 30, 32, 34, and 67) were found to have binding performance that was equivalent to or better than V9 (see FIG. 9).

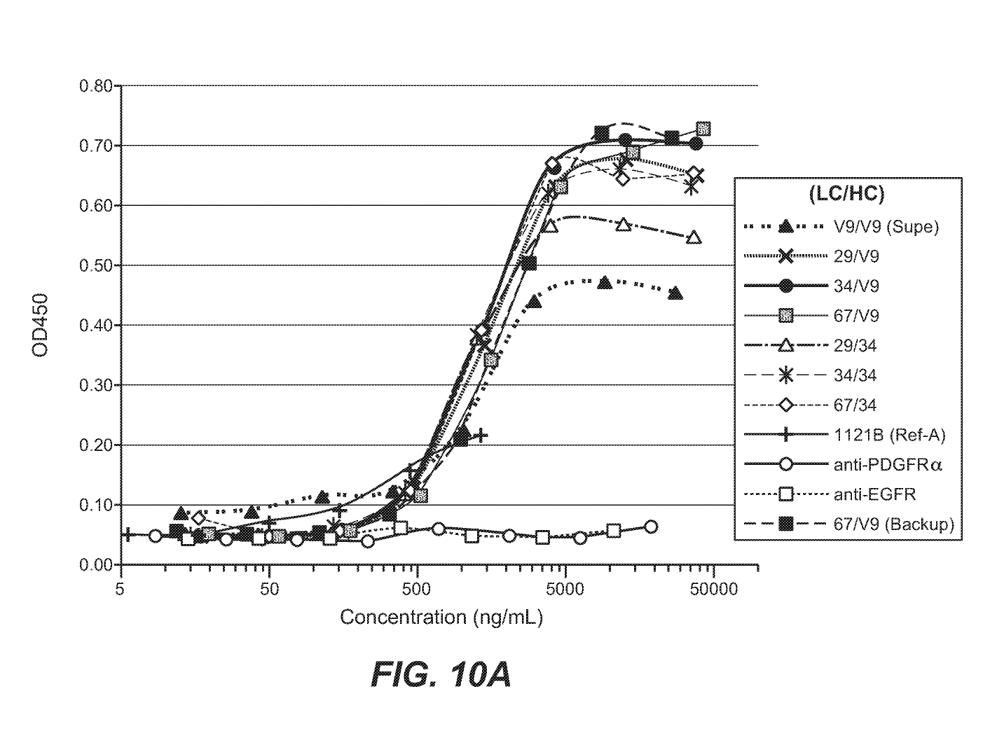

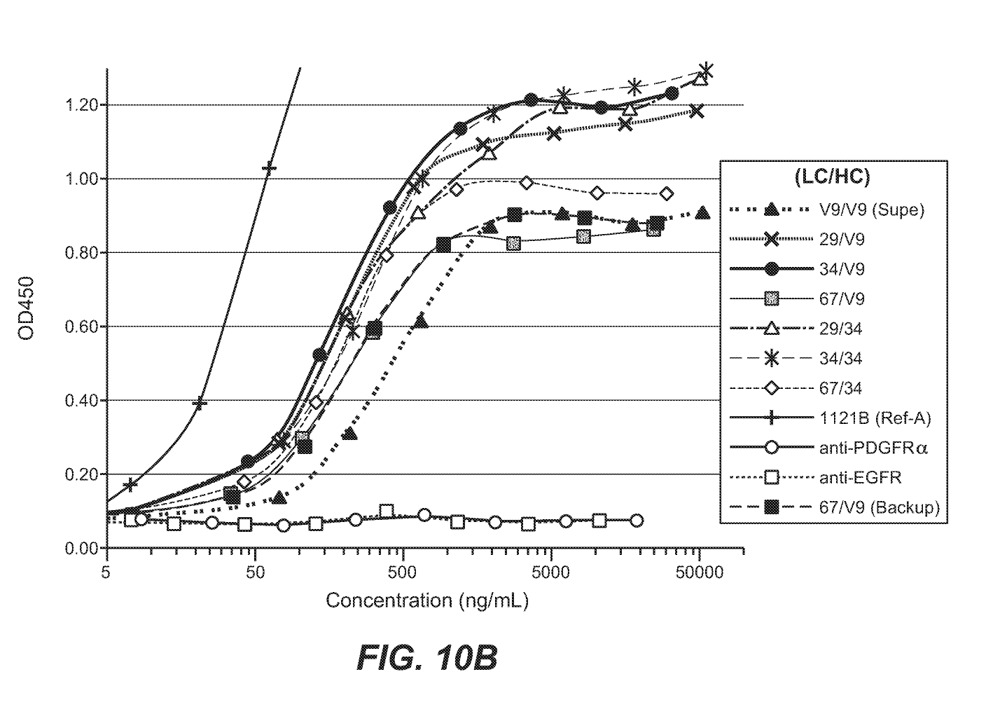

Further ELISAs were performed using full-length IgG clones comprising the following light chain/heavy chain combinations: V9/V9, 29/V9 (LC/HC), 34/V9 (LC/HC), 67/V9 (LC/HC), 29/34 (LC/HC), 34/34(LC/HC), and 67/34 (LC/HC). 1121B and two negative control antibodies were also tested. Serial dilutions of each clone, 1121B, and two negative control antibodies were captured with VEGFR2-Fc in wells of a microtiter dish. The amount of captured antibody in each well was quantified using an anti-human kappa-HRP-conjugated secondary antibody. The HRP-conjugated secondary antibody was added to the wells, and, following an incubation, excess secondary antibody was washed away. TMB was added to the wells, and following incubation, the reaction was stopped, and HRP activity was measured by monitoring the increase in absorbance at 450 nm (FIG. 10A). 34/V9 (LC/HC), 34/34 (LC/HC), and 67/V9 (LC/HC), demonstrated the strongest binding to VEGFR2 of the clones tested.

A third set of ELISAs were performed in which full-length IgG clones of V9/V9, 29/V9 (LC/HC), 34/V9 (LC/HC), 67/V9 (LC/HC), 29/34 (LC/HC), 34/34(LC/HC), 67/34 (LC/HC), 1121B, and two negative control antibodies were captured with anti-Fd in wells of a microtiter dish. The amount of captured antibody in each well was quantified using an AP-conjugated VEGFR2. Following an incubation, excess VEGFR2-AP was washed away. Alkaline phosphatase substrate was added to the wells, and following incubation, the reaction was stopped, and AP activity was measured by monitoring the increase in absorbance at 405 nm (FIG. 10B). 34/V9 (LC/HC), 34/34 (LC/HC), 29/V9 (LC/HC), and 29/34 (LC/HC) demonstrated the strongest binding to VEGFR2 of the clones tested.

SPR was performed on V9/V9 (LC/HC), 34/34 (LC/HC), 34/V9 (LC/HC), 67/34 (LC/HC), 67/V9 (LC/HC), 29/34 (LC/HC) and 29/V9 (LC/HC). The results are shown in Tables 8A-8C below.

TABLE 8A

| LC/HC | ka (1/(M * s)) | kd (1/s) | K_{D} (M) |
|---|---|---|---|
| V9/V9 | 6.53 × 10⁵ | 6.37 × 10⁻⁴ | 9.76 × 10⁻¹⁰ |
| 34/34 | 8.25 × 10⁵ | 3.89 × 10⁻⁴ | 4.71 × 10⁻¹⁰ |
| 34/V9 | 7.48 × 10⁵ | 2.72 × 10⁻⁴ | 3.64 × 10⁻¹⁰ |
| 67/34 | 5.49 × 10⁵ | 2.63 × 10⁻⁴ | 4.79 × 10⁻¹⁰ |
| 67/V9 | 6.41 × 10⁵ | 3.17 × 10⁻⁴ | 4.94 × 10⁻¹⁰ |
| 29/34 | 7.97 × 10⁵ | 3.36 × 10⁻⁴ | 4.26 × 10⁻¹⁰ |
| 29/V9 | 9.64 × 10⁵ | 2.10 × 10⁻⁴ | 2.18 × 10⁻¹⁰ |

TABLE 8B

| LC/HC | ka (1/(M * s)) | kd (1/s) | K_{D} (M) |
|---|---|---|---|
| V9/V9 | 6.9 × 10⁵ | 3.6 × 10⁻⁴ | 5.1 × 10⁻¹⁰ |
| 34/34 | 8.3 × 10⁵ | 2.4 × 10⁻⁴ | 2.9 × 10⁻¹⁰ |
| 34/V9 | 4.4 × 10⁵ | 2.3 × 10⁻⁴ | 5.3 × 10⁻¹⁰ |
| 67/34 | 5.8 × 10⁵ | 1.5 × 10⁻⁴ | 2.5 × 10⁻¹⁰ |
| 67/V9 | 6.9 × 10⁵ | 2.0 × 10⁻⁴ | 2.8 × 10⁻¹⁰ |
| 29/34 | 6.0 × 10⁵ | 1.6 × 10⁻⁴ | 2.7 × 10⁻¹⁰ |
| 29/V9 | 7.2 × 10⁵ | 4.8 × 10⁻⁵ | 6.7 × 10⁻¹¹ |

TABLE 8C

| LC/HC | ka (1/(M * s)) | kd (1/s) | K_{D} (M) |
|---|---|---|---|
| V9/V9 | 8.55 × 10⁵ | 5.28 × 10⁻⁴ | 6.17 × 10⁻¹⁰ |
| 34/34 | 1.22 × 10⁶ | 2.55 × 10⁻⁴ | 2.09 × 10⁻¹⁰ |
| 34/V9 | 9.23 × 10⁵ | 2.37 × 10⁻⁴ | 2.57 × 10⁻¹⁰ |
| 67/34 | 7.98 × 10⁵ | 6.53 × 10⁻⁵ | 8.19 × 10⁻¹¹ |
| 67/V9 | 6.69 × 10⁵ | 9.00 × 10⁻⁵ | 1.34 × 10⁻¹⁰ |
| 29/34 | 8.45 × 10⁵ | 9.22 × 10⁻⁵ | 1.09 × 10⁻¹⁰ |
| 29/V9 | 9.32 × 10⁵ | 2.23 × 10⁻⁴ | 2.39 × 10⁻¹⁰ |
| 1121B | 7.52 × 10⁵ | 9.62 × 10⁻⁴ | 1.28 × 10⁻⁹ |
| (Ref A) | | | |

29/V9 (LC/HC) consistently demonstrated a stronger K_{D} than the other clones tested. 67/34 (LC/HC) consistently demonstrated the lowest k_{off}, and 34/34 (LC/HC) consistently demonstrated the highest kₒₙ. 34/34 (LC/HC), 34/V9 (LC/HC), 67/34 (LC/HC), 67/V9 (LC/HC), 29/34 (LC/HC) and 29/V9 (LC/HC) all show improvement in dissociation as compared to V9/V9 (LC/LC).

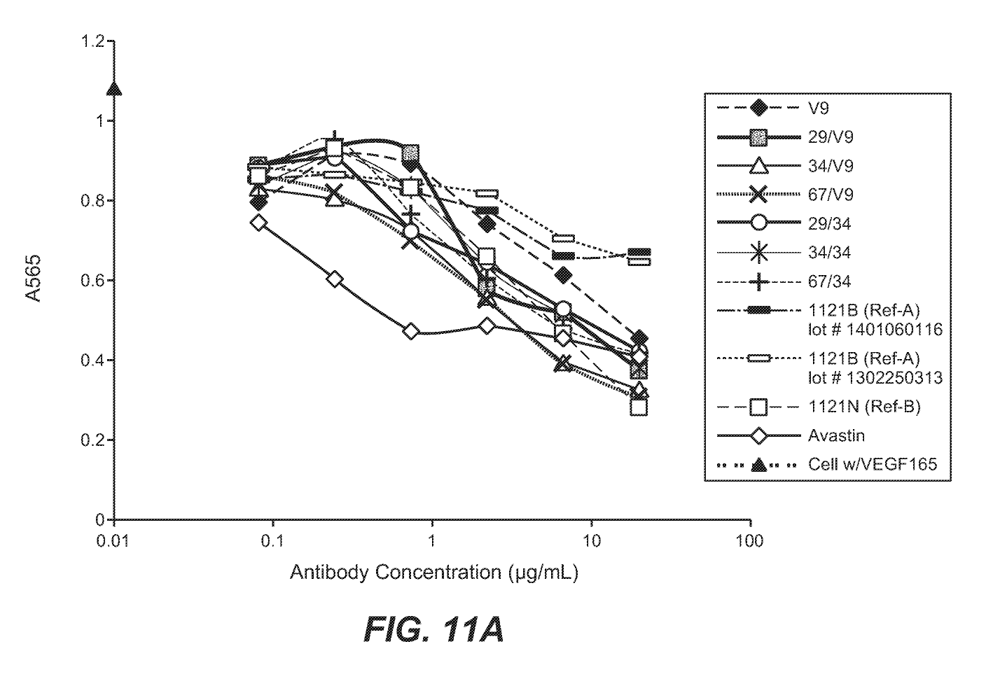

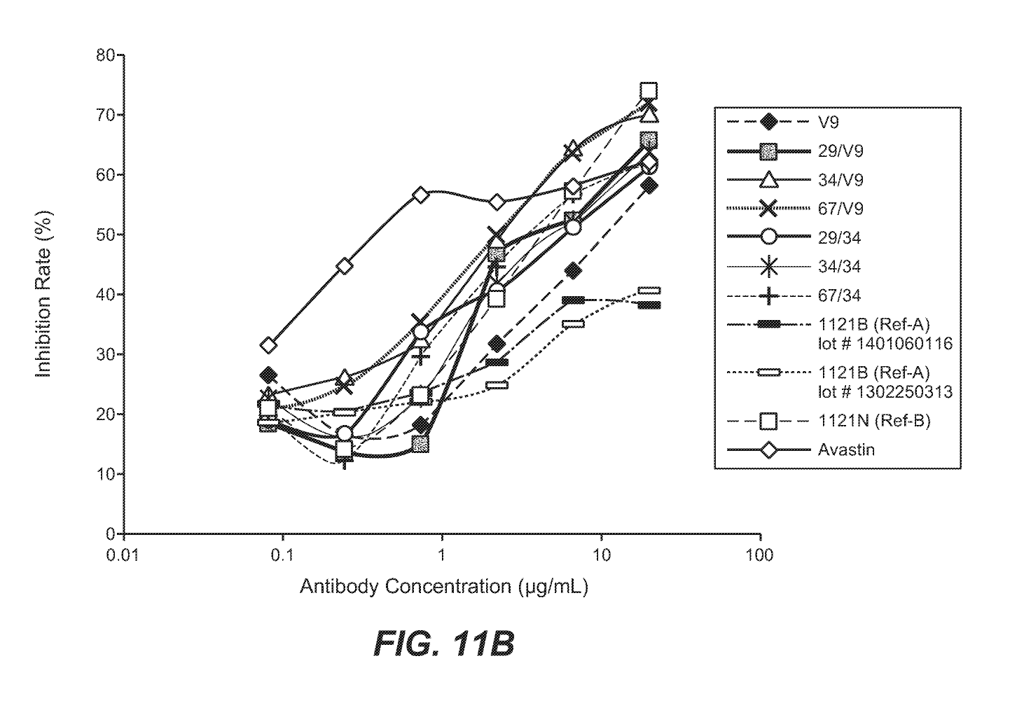

HUVEC proliferation assays were performed as described above using clones V9/V9 (LC/HC), 29/V9 (LC/HC), 34/V9 (LC/HC), 67/V9 (LC/HC), 29/34 (LC/HC), 34/34 (LC/HC), 67/34 (LC/HC), 1121B (Ref A), 1121N (Ref B), and Avastin. As shown in FIGS. 11A and 11B, 34/V9 (LC/HC), 67/V9 (LC/HC), and 29/V9 (LC/HC) exhibited the strongest inhibition of HUVEC proliferation of all clones tested.

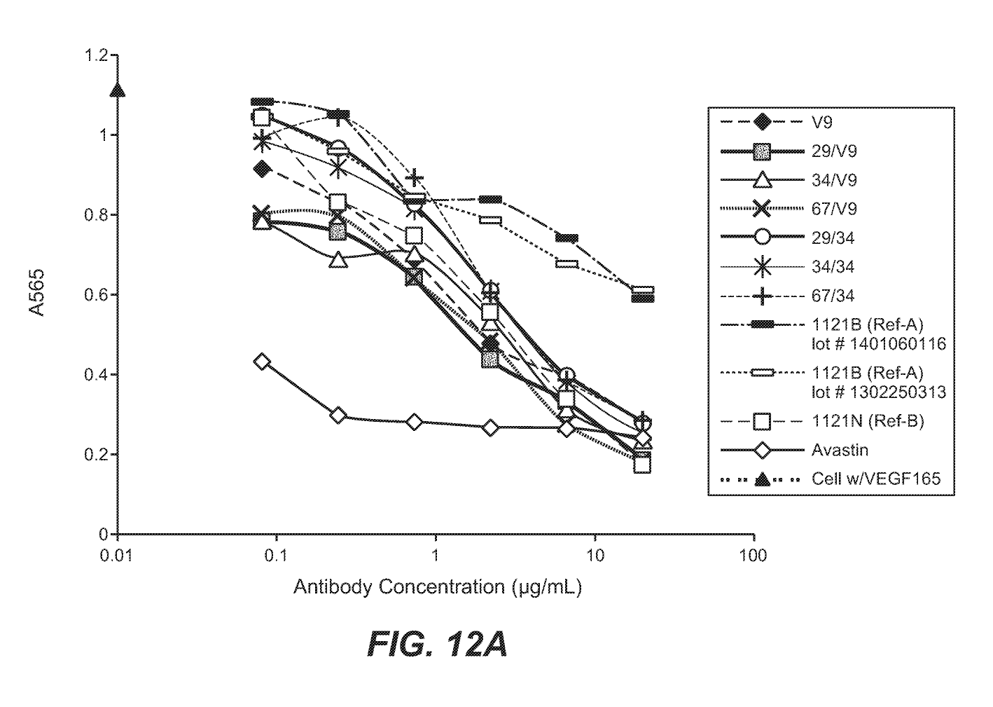

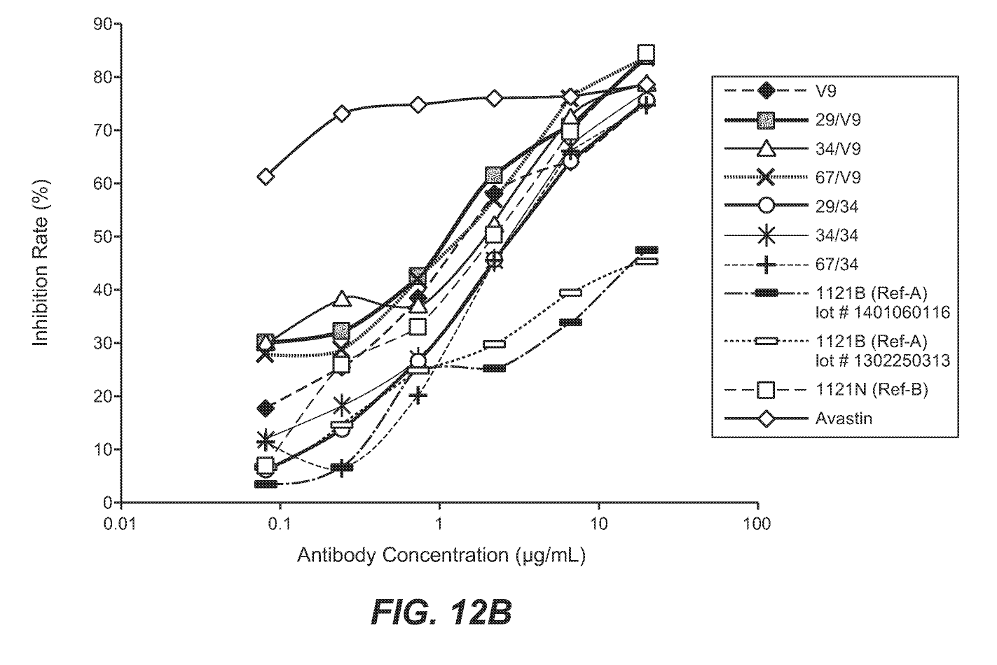

HUVEC survival assays were performed as described above using clones V9/V9 (LC/HC), 29/V9 (LC/HC), 34/V9 (LC/HC), 67/V9 (LC/HC), 29/34 (LC/HC), 34/34 (LC/HC), 67/34 (LC/HC), 1121B (Ref A), 1121N (Ref B), and Avastin. As shown in FIGS. 12A and 12B, 34/V9 (LC/HC), 67/V9 (LC/HC), and 29/V9 (LC/HC) exhibited the strongest inhibition of HUVEC survival of all clones tested.

HUVEC tube formation assays were performed as described above using clones V9/V9 (LC/HC), 29/V9 (LC/HC), 34/V9 (LC/HC), 67/V9 (LC/HC), 29/34 (LC/HC), 34/34 (LC/HC), 67/34 (LC/HC), 1121B (Ref A), 1121N (Ref B), and Avastin. 1121N (Ref B) exhibited the strongest inhibition of HUVEC tube formation. V9/V9 (LC/HC), 29/V9 (LC/HC), 67/V9 (LC/HC), and 1121B (Ref A) exhibited stronger HUVEC tube formation than 29/34 (LC/HC). (Data not shown.)

HUVEC sprouting assays were performed as described above using clones V9/V9 (LC/HC), 29/V9 (LC/HC), 34/V9 (LC/HC), 67/V9 (LC/HC), 29/34 (LC/HC), 34/34 (LC/HC), 67/34 (LC/HC), 1121B (Ref A), 1121N (Ref B), and Avastin. 1121N (Ref B) exhibited the strongest inhibition of HUVEC sprouting. 67/34 (LC/HC) and 34/34 (LC/HC) exhibited stronger inhibition of HUVEC sprouting than 1121B (Ref A). 1121B (Ref A) exhibited stronger inhibition of HUVEC sprouting than 34/V9 (LC/HC). 34/V9 (LC/HC) exhibited stronger inhibition of HUVEC sprouting than V9/V9 (LC/HC) and 67/34 (LC/HC), which exhibited stronger sprouting inhibition than 29/34 (LC/HC). 29/V9 (LC/HC) did not demonstrate strong inhibition of HUVEC sprouting. (Data not shown.)

Clones 29, 34, and 67 were selected and used as the basis for generating CDR-H1/CDR-H1/CDR-H2 libraries. CDR-H1/CDR-H1/CDR-H2 nucleic acid libraries were generated via PCR, cloned into a phage display vector, and transformed into E. coli to produce a library of phages. After two rounds of panning, seven clones, i.e., 80A, 80B, 86A, 86B, 88, 109, 110A and 110B, were screened via ELISA and found to have improved binding properties.

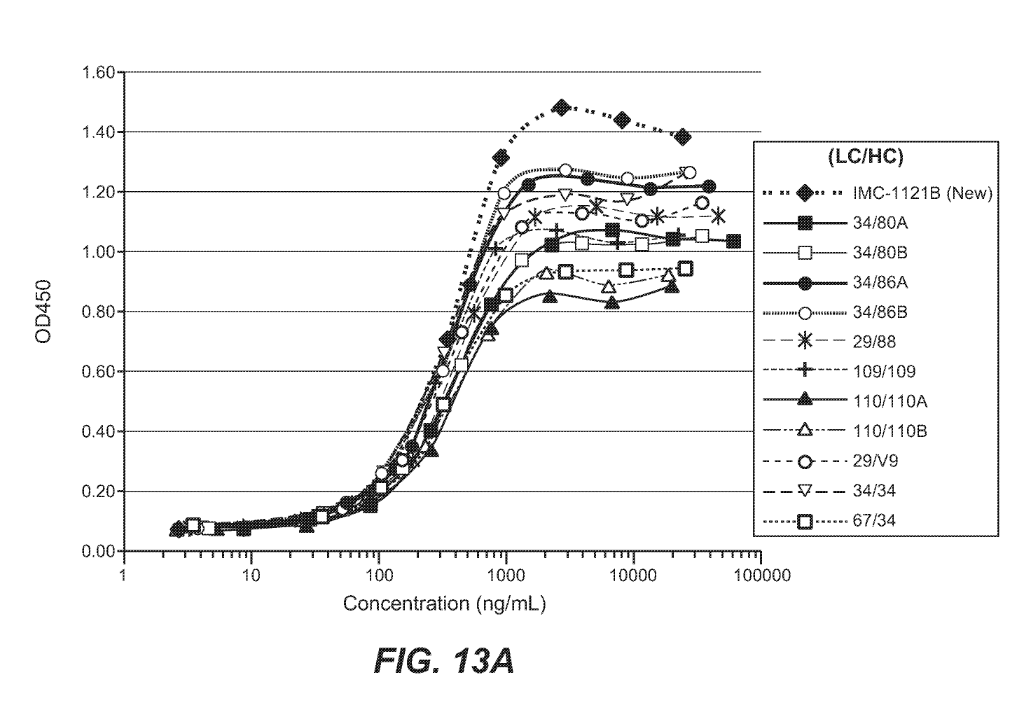

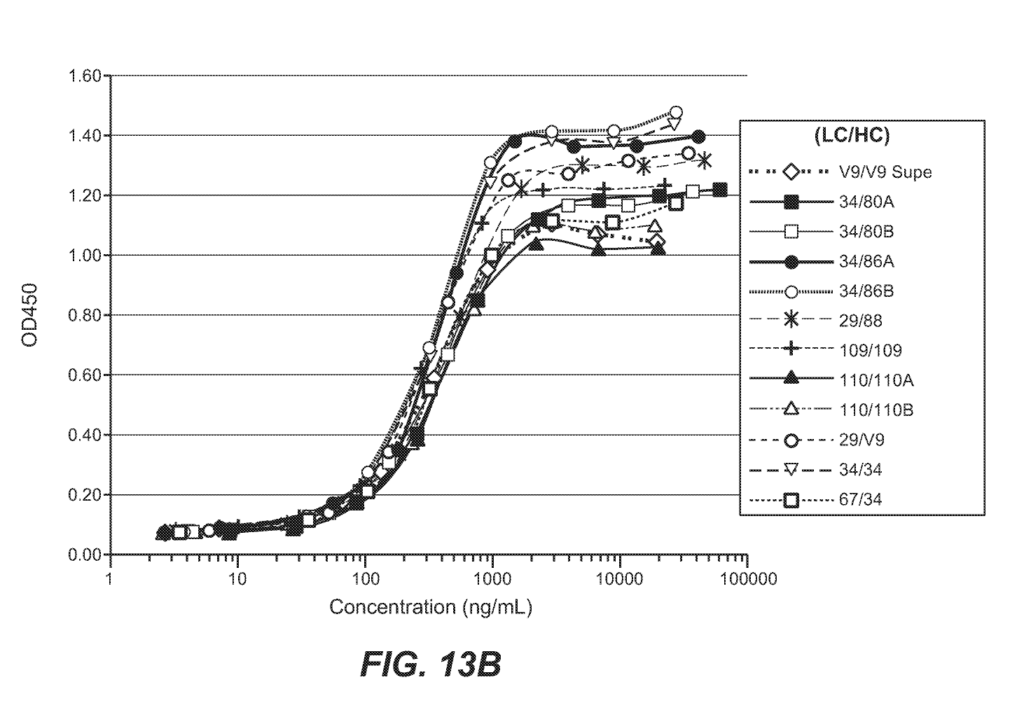

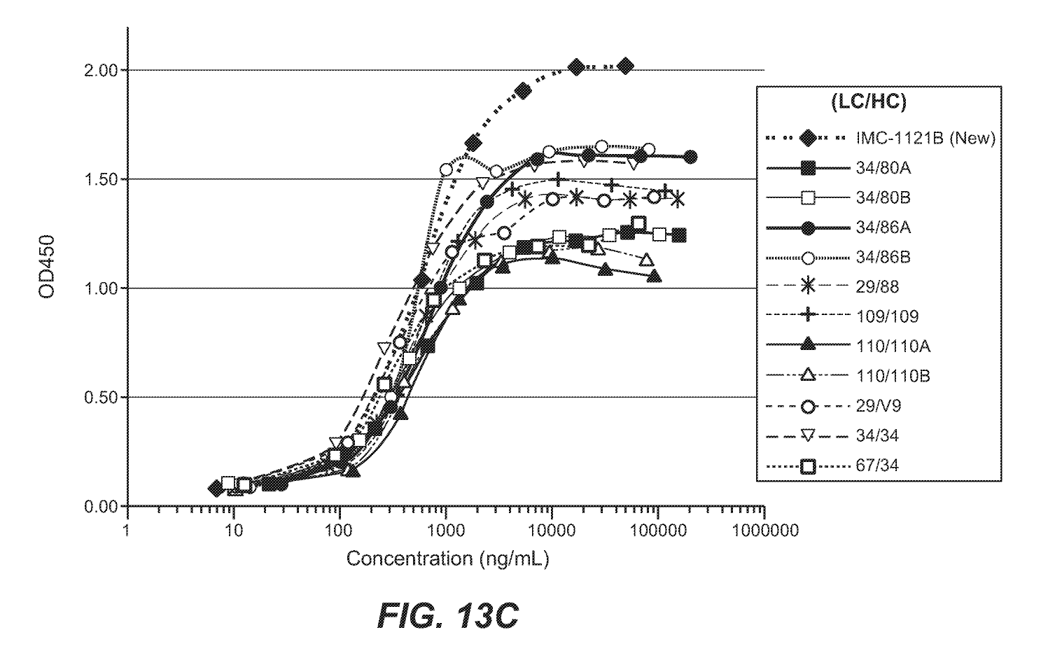

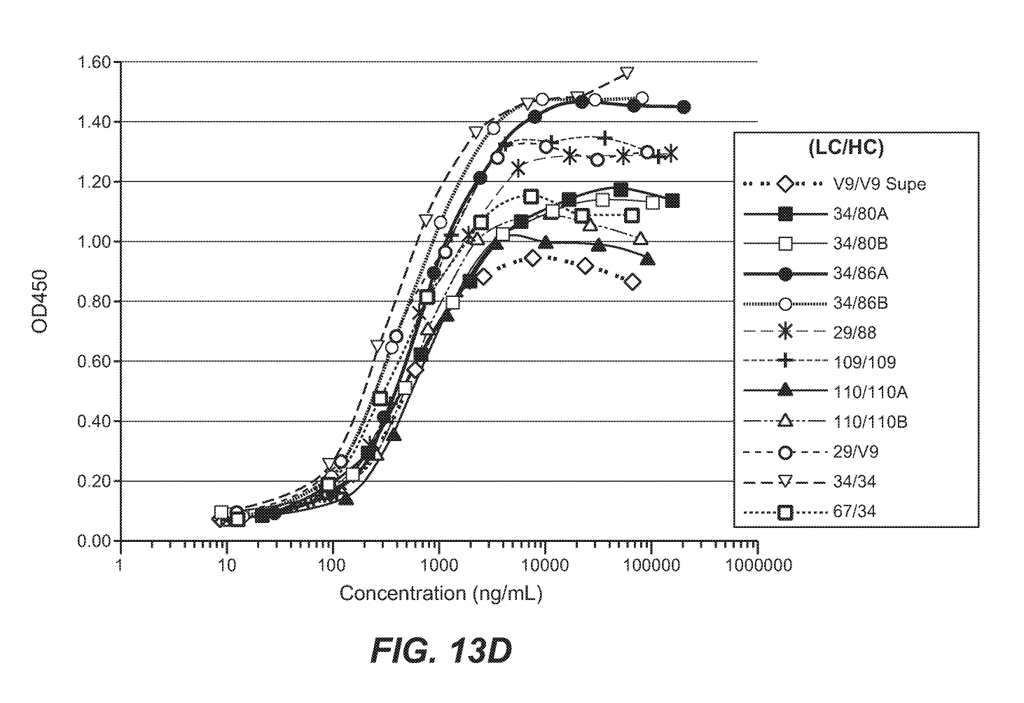

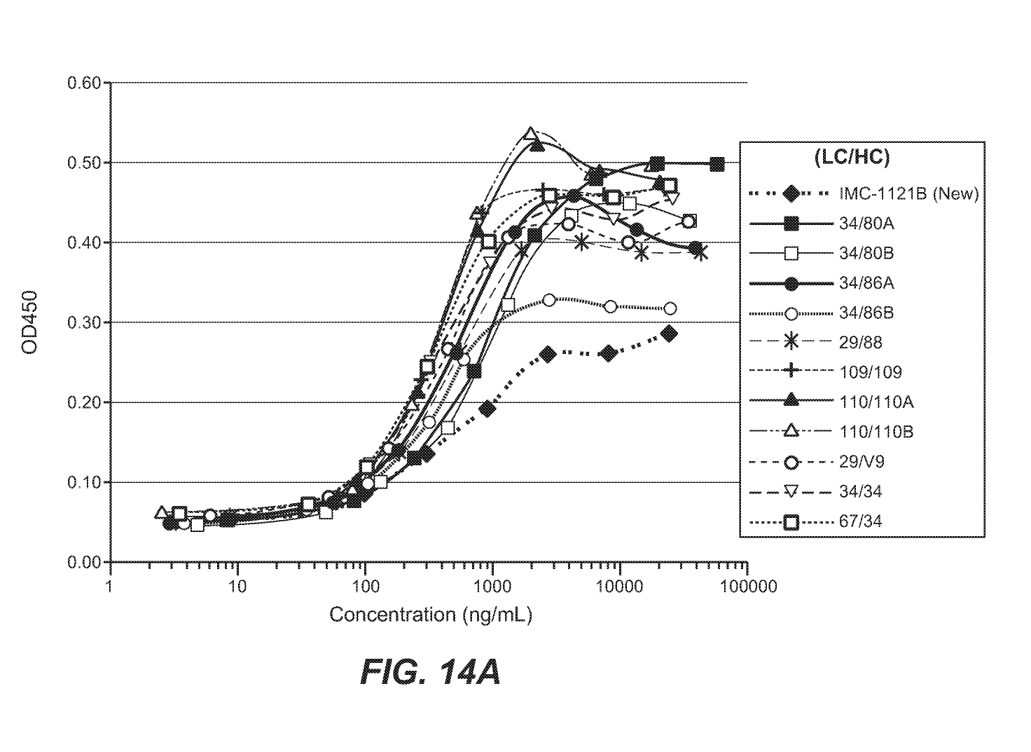

Additional ELISAs were performed with anti-VEGFR2 antibody clones comprising the following light chain/heavy chain combinations: V9/V9 (LC/HC), 34/80A (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 29/V9 (LC/HC), 34/34 (LC/HC), and 67/34 (LC/HC) as follows: Serial dilutions of each clone and 1121B (Ref A) were captured with anti-Fc antibody in wells of a microtiter dish. The amount of captured antibody in each well was quantified using AP-conjugated VEGFR. Following an incubation, excess VEGFR2-AP was washed away. Alkaline phosphatase substrate was added to the wells, and following incubation, the reaction was stopped, and AP activity was measured by monitoring the increase in absorbance at 405 nm (FIGS. 13A and 13B). 1121B (Ref A) demonstrated the strongest binding to VEGFR2, followed by 34/86A (LC/HC), 34/86B (LC/HC), and 34/34 (LC/HC), followed by 29/88 (LC/HC), 109/109 (LC/HC), and 29/v9 (LC/HC).

Further ELISAs were performed in which V9/V9 (LC/HC), 34/80A (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 29/V9 (LC/HC), 34/34 (LC/HC), 67/34 (LC/HC), and 1121B were captured with anti-Fd in wells of a microtiter dish. The amount of captured antibody in each well was quantified using AP-conjugated VEGFR. Following an incubation, excess VEGFR2-AP was washed away. Alkaline phosphatase substrate was added to the wells, and following incubation, the reaction was stopped, and AP activity was measured by monitoring the increase in absorbance at 405 nm (FIGS. 13C and 13D). 1121B (Ref A) demonstrated the strongest binding to VEGFR2, followed by 34/86A (LC/HC), 34/86B (LC/HC), and 34/34 (LC/HC), followed by 29/88 (LC/HC), 109/109 (LC/HC), and 29/v9 (LC/HC). All clones tested demonstrated stronger binding to VEGFR2 than V9 (see FIG. 13D).

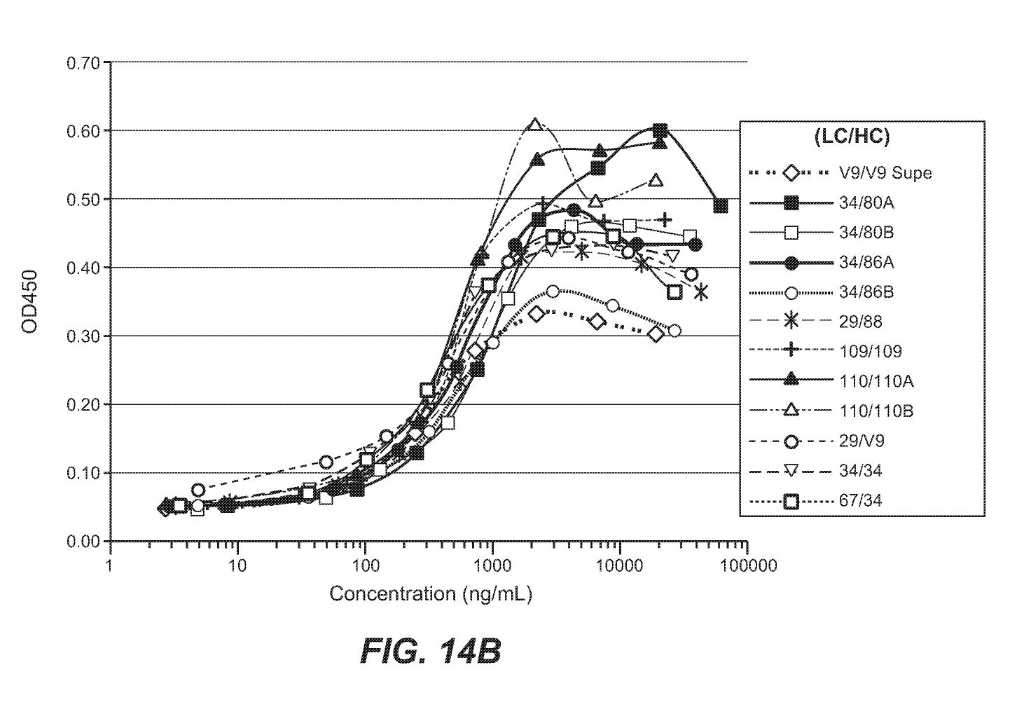

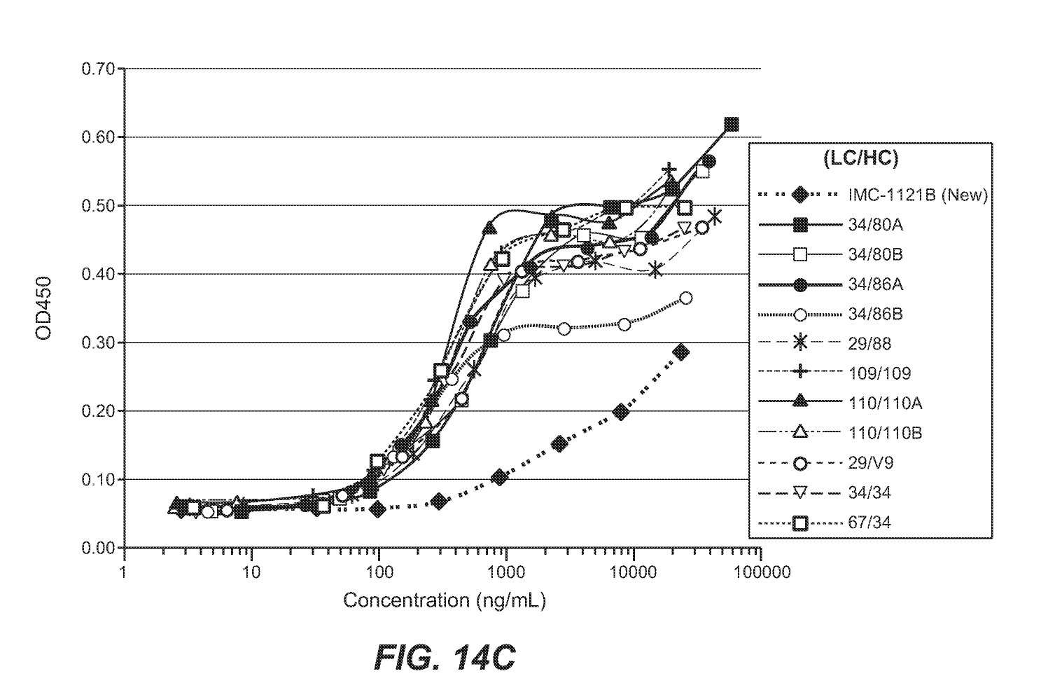

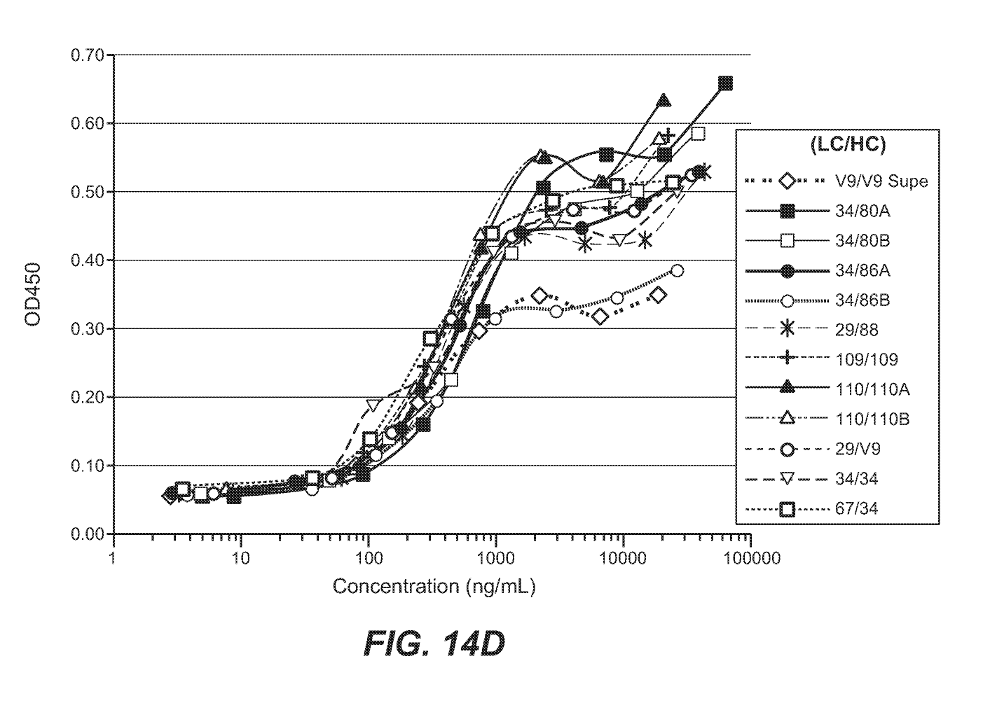

A third set of ELISAs were performed. Briefly, V9/V9 (LC/HC), 34/80A (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 29/V9 (LC/HC), 34/34 (LC/HC), 67/34 (LC/HC), and 1121B were captured with VEGFR2-Fc in wells of a microtiter dish. The amount of captured antibody in each well was quantified using an anti-human kappa-HRP-conjugated secondary antibody. The HRP-conjugated secondary antibody was added to the wells, and, following an incubation, excess secondary antibody was washed away. TMB was added to the wells, and following incubation, the reaction was stopped, and HRP activity was measured by monitoring the increase in absorbance at 450 nm (FIGS. 14A and 14B). 34/80A (LC/HC), 34/80B (LC/HC), 34/86A (LC/HC), 29/88 (LC/HC), 109/109 (LC/HC), 110A/110A (LC/HC), 110B/110B (LC/HC), 29/V9 (LC/HC), 34/34 (LC/HC), and 67/34 (LC/HC) demonstrated the strongest binding to VEGFR2, followed by V9/V9 (LC/HC) and 34/86B (LC/HC). All clones tested demonstrated stronger binding to VEGFR2 than 1121B (Ref A).

Further ELISAs were performed in which Briefly, V9/V9 (LC/HC), 34/80A (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 29/V9 (LC/HC), 34/34 (LC/HC), 67/34 (LC/HC), and 1121B were captured with biotinylated VEGFR2-Fc immobilized in wells of a NeutrAvidin-coated microtiter dish. The amount of captured antibody in each well was quantified using an anti-human kappa-HRP-conjugated secondary antibody. The HRP-conjugated secondary antibody was added to the wells, and, following an incubation, excess secondary antibody was washed away. TMB was added to the wells, and following incubation, the reaction was stopped, and HRP activity was measured by monitoring the increase in absorbance at 450 nm (FIGS. 14C and 14D). 34/80A (LC/HC), 34/80B (LC/HC), 34/86A (LC/HC), 29/88 (LC/HC), 109/109 (LC/HC), 110A/110A (LC/HC), 110B/110B (LC/HC), 29/V9 (LC/HC), 34/34 (LC/HC), and 67/34 (LC/HC) demonstrated the strongest binding to VEGFR2, followed by V9/V9 (LC/HC) and 34/86B (LC/HC). All clones tested demonstrated stronger binding to VEGFR2 than 1121B (Ref A).

SPR was performed on V9/V9 (LC/HC), 34/80A (LC/HC), 110A/110A (LC/HC), 34/80B (LC/HC), 110B/110B (LC/HC), and 29/V9 (LC/HC). The results are shown in Tables 9A and 9B below.

TABLE 9A

| LC/HC | ka (1/(M * s)) | kd (1/s) | K_{D} (M) |
|---|---|---|---|
| V9/V9 | 1.1 × 10⁶ | 8.49 × 10⁻⁴ | 7.74 × 10⁻¹⁰ |
| 34/V9 | 7.84 × 10⁵ | 2.27 × 10⁻⁴ | 2.89 × 10⁻¹⁰ |
| 34/80A | 2.15 × 10⁵ | 2.98 × 10⁻⁴ | 1.38 × 10⁻⁹ |

TABLE 9B

| LC/HC | ka (1/(M * s)) | kd (1/s) | K_{D} (M) |
|---|---|---|---|
| V9/V9 | 7.90 × 10⁵ | 8.82 × 10⁻⁴ | 1.12 × 10⁻⁹ |
| 34/80A | 4.12 × 10⁴ | 1.31 × 10⁻⁴ | 3.17 × 10⁻⁹ |
| 110A/110A | 2.28 × 10⁶ | 1.88 × 10⁻⁴ | 8.26 × 10⁻¹¹ |
| 34/80B | 4.88 × 10⁴ | 2.44 × 10⁻⁴ | 5.01 × 10⁻⁰⁹ |
| 110B/110B | 1.70 × 10⁶ | 2.19 × 10⁻⁴ | 1.29 × 10⁻¹⁰ |
| 29/V9 | 8.67 × 10⁵ | 3.47 × 10⁻⁴ | 4.01 × 10⁻¹⁰ |

110A/110A (LC/HC) demonstrates improved kₐ, k_{d}, and K_{D} as compared to the other clones tested. 110A/110B (LC/HC) binding is slightly lower than 110A/110A (LC/HC). 34/80B (LC/HC) binding is slightly lower than 34/80A (LC/HC).

SPR kinetics analyses were performed on V9/V9 (LC/HC), 34/80A (LC/HC), 110A/110A (LC/HC), 34/80B (LC/HC), 110B/110B (LC/HC), and 29/V9 ((LC/HC). The results are shown in Tables 10A-10C below.

TABLE 10A

| LC/HC | ka (1/(M * s)) | kd (1/s) | K_{D} (M) | Rmax |
|---|---|---|---|---|
| V9/V9 | 2.02 × 10⁶ | 3.94 × 10⁻⁴ | 1.95 × 10⁻¹⁰ | 42 |
| 110A/110A | 1.80 × 10⁶ | 8.76 × 10⁻⁶ | 4.88 × 10⁻¹² | 107 |
| 34/86 | 6.12 × 10⁵ | 1.00 × 10⁻⁴ | 1.64 × 10⁻¹⁰ | 79 |
| 34/80A | 6.12 × 10⁵ | 5.75 × 10⁻⁵ | 9.39 × 10⁻¹¹ | 40 |
| 109/109 | 1.67 × 10⁶ | 7.08 × 10⁻⁵ | 4.25 × 10⁻¹¹ | 118 |
| 29/88 | 8.90 × 10⁵ | 7.80 × 10⁻⁵ | 8.76 × 10⁻¹¹ | 63 |

TABLE 10B

| | | | | | Fold |
|---|---|---|---|---|---|
| LC/HC | ka (1/(M * s)) | kd (1/s) | K_{D} (M) | Rmax | KD |
| V9/V9 | 9.89 × 10⁵ | 3.47 × 10⁻⁴ | 3.51 × 10⁻¹⁰ | 62.9 | 1 |
| 110A/110A | 1.64 × 10⁶ | 4.78 × 10⁻⁵ | 2.92 × 10⁻¹¹ | 92.2 | 12 |
| 109/109 | 1.71 × 10⁶ | 7.88 × 10⁻⁵ | 4.61 × 10⁻¹¹ | 108.4 | 8 |
| 67/V9 | 6.20 × 10⁵ | 6.03 × 10⁻⁵ | 9.72 × 10⁻¹¹ | 73.3 | 4 |
| 34/34 | 9.30 × 10⁵ | 1.39 × 10⁻⁴ | 1.49 × 10⁻¹⁰ | 69.2 | 2 |

TABLE 10C

| | ka | | | | Fold |
|---|---|---|---|---|---|
| LC/HC | (1/(M * s)) | kd (1/s) | K_{D} (M) | Rmax | KD |
| V9/V9 | 9.98 × 10⁵ | 4.24 × 10⁻⁴ | 4.25 × 10⁻¹⁰ | 70.69 | 1.0 |
| 110A/110A | 1.35 × 10⁶ | 1.21 × 10⁻⁴ | 8.92 × 10⁻¹¹ | 104.93 | 4.8 |
| 109/109 | 1.54 × 10⁶ | 8.19 × 10⁻⁵ | 5.33 × 10⁻¹¹ | 116.29 | 8.0 |
| 67/V9 | 6.14 × 10⁵ | 7.88 × 10⁻⁵ | 1.28 × 10⁻¹⁰ | 91.8 | 3.3 |
| 34/34 | 8.92 × 10⁵ | 1.38 × 10⁻⁴ | 1.54 × 10⁻¹⁰ | 80.53 | 2.8 |

110A/110A (LC/HC) and 109/109 (LC/HC) consistently demonstrated higher kₐ and lower k_{d} than V9/V9. Accordingly, the K_{D}s of 110A/110A (LC/HC) and 109/109 (LC/HC) were lower. In addition, Rmax for 110A/110A (LC/HC) and 109/109 (LC/HC) was consistently higher.

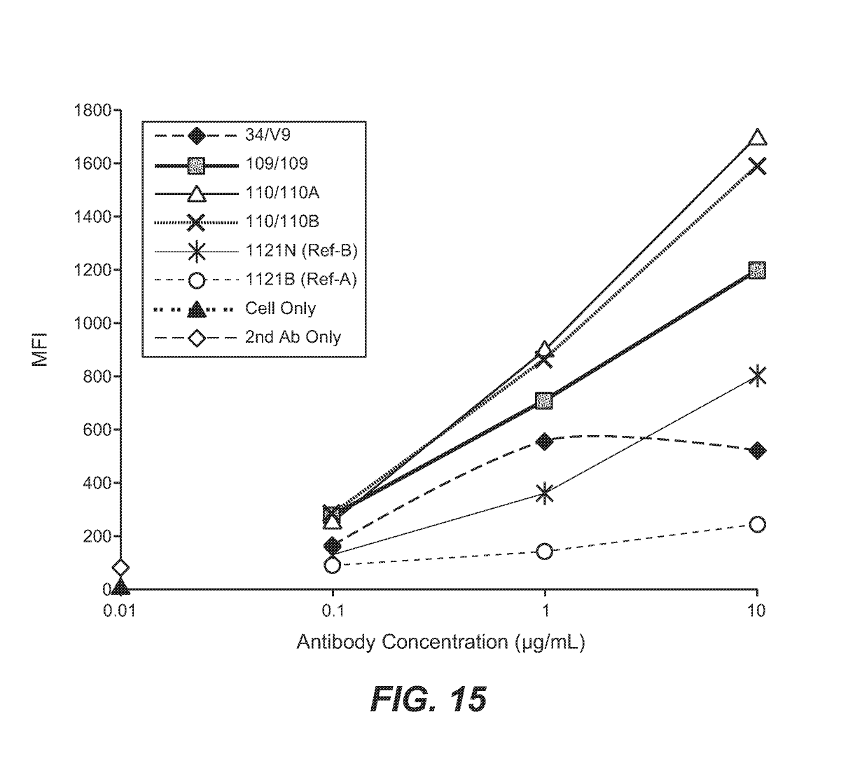

Clones 34/V9 (LC/HC), 109/109 (LC/HC), 110A/110A (LC/HC), and 110B/110B were assessed for their abilities to bind HUVEC whole cells as follows: HUVECs were harvested, washed with PBS, and fixed with paraformaldehyde for 30 minutes at 4° C. The cells were then washed with FACS buffer and added to wells of a microtiter plate, which was then centrifuged. 34/V9 (LC/HC), 109/109 (LC/HC), 110A/110A (LC/HC), or 110B/110B was added to each well and incubated for 30 minutes at 4° C. After the wells were washed twice with FACS buffer, biotinylated rabbit anti-human IgG was added to each well and incubated for 30 minutes at 4° C. Following two more washes with FACS buffer, streptavidin-PE (i.e., streptavidin conjugated to phycoerythrin) was added to each well and incubated. The wells were washed twice with FACS buffer, fixed with 4% paraformaldehyde, and centrifuged. The supernatants were discarded, and the cells were resuspended in FACS buffer and analyzed via flow cytometry. As shown in FIG. 15, clones 110A/110A (LC/HC), 110B/110B (LC/HC), and 109/109 (LC/HC) exhibited stronger binding to HUVECS than 1121N, which exhibited stronger binding to HUVECs than 34/V9 (LC/HC). All clones tested exhibited stronger binding to HUVECs than 1121B. Such results correspond to the ELISA results described above.

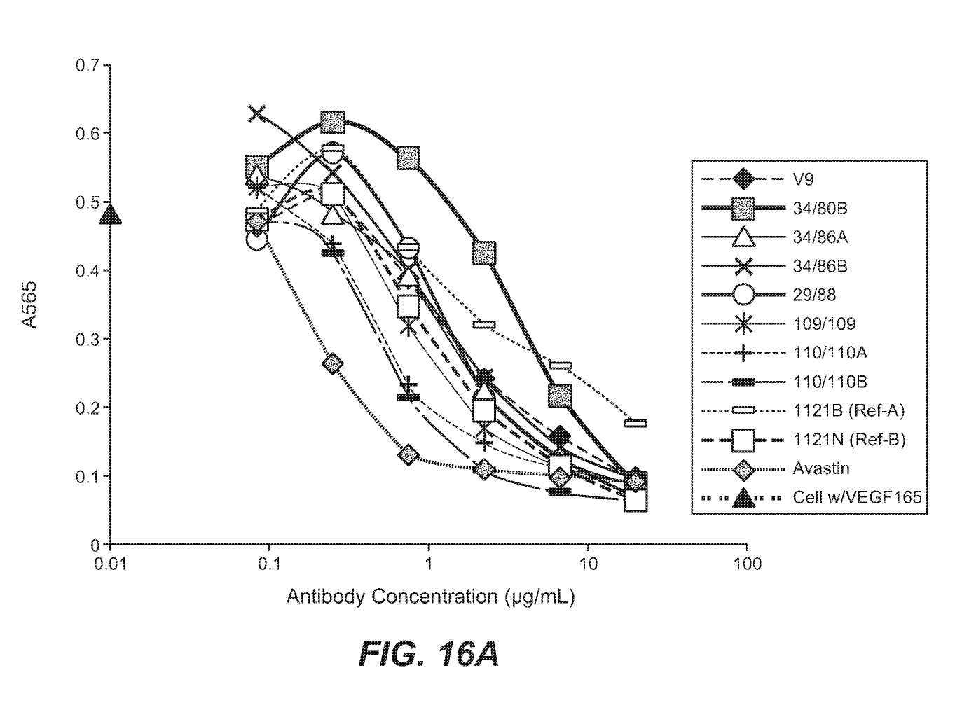

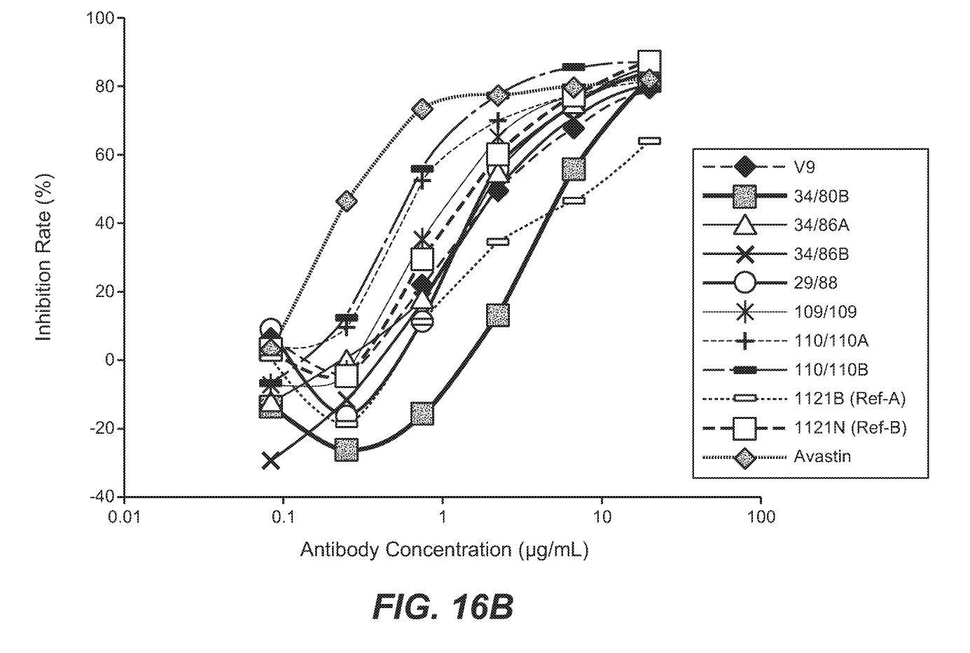

HUVEC proliferation assays were performed as described above using clones V9/V9 (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 1121B (Ref A), 1121N (Ref B), and Avastin. As shown in FIGS. 16A and 16B, 110A/110A (LC/HC) and 110B/110B (LC/HC) exhibited the strongest inhibition of HUVEC proliferation of all clones tested, including 1121B (Ref A) and 1121N (Ref B). 109/109 (LC/HC) exhibited stronger inhibition of HUVEC proliferation than 1121N (Ref B), which exhibited stronger inhibition of HUVEC proliferation than 34/86A (LC/HC), 34/86B (LC/HC), 29/88 (LC/HC), and V9/V9 (LC/HC). 34/86A (LC/HC), 34/86B (LC/HC), 29/99 (LC/HC), and V9/V9 (LC/HC) exhibited stronger inhibition of HUVEC proliferation than 1121B (Ref A), which exhibited stronger inhibition of HUVEC proliferation than 34/80A (LC/HC).

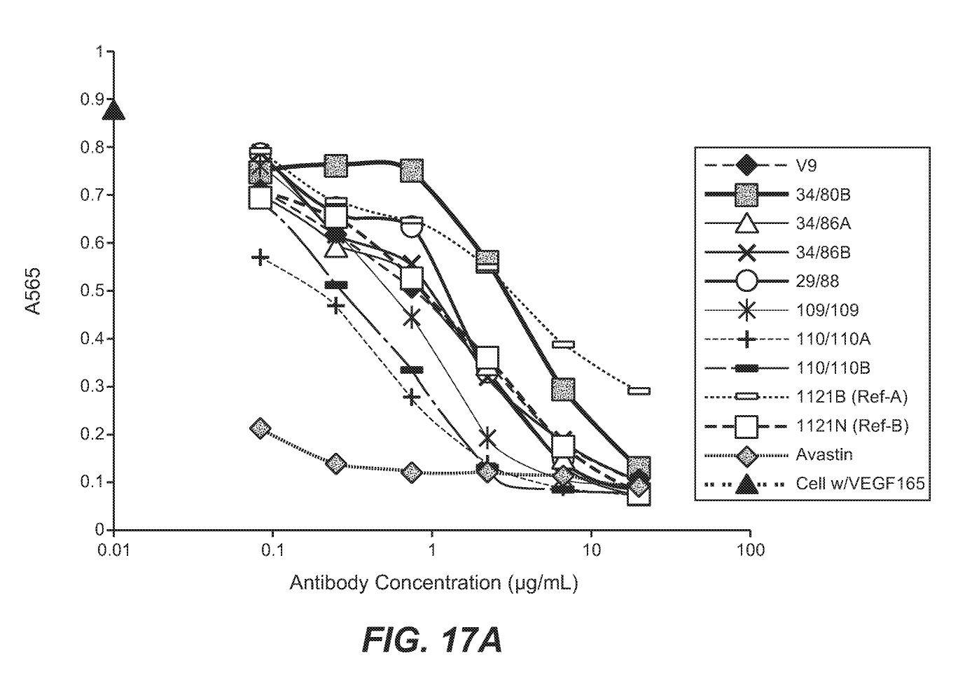

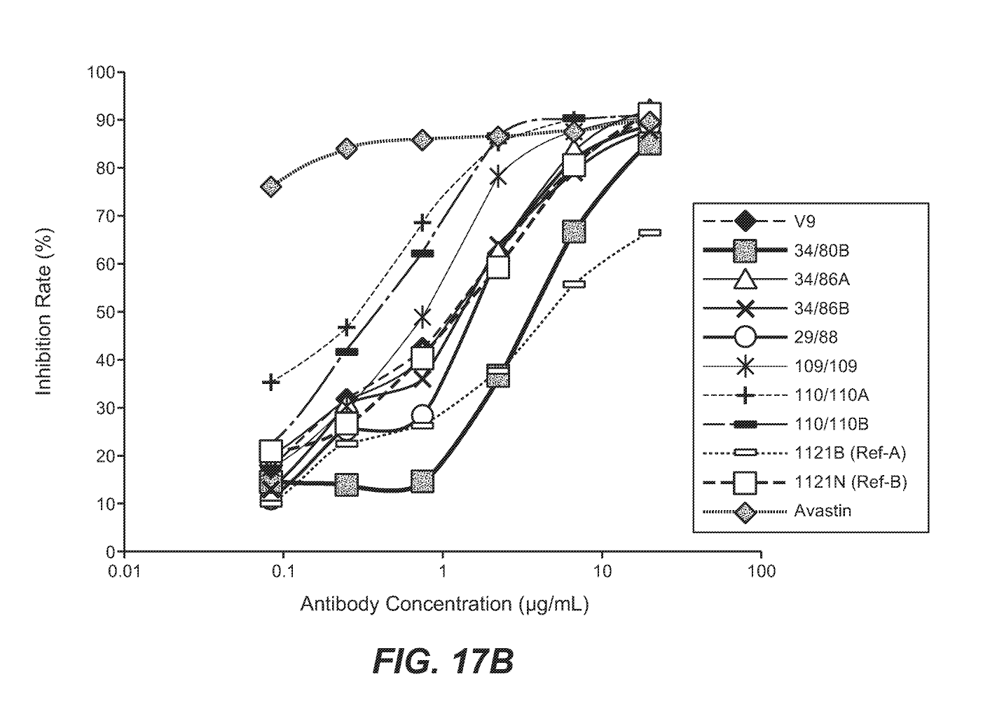

HUVEC survival assays were performed as described above using clones V9/V9 (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 1121B (Ref A), 1121N (Ref B), and Avastin. As shown in FIGS. 17A and 17B, 110A/110A (LC/HC) and 110B/110B (LC/HC) exhibited the strongest inhibition of HUVEC survival of all clones tested, including 1121B (Ref A) and 1121N (Ref B). 109/109 (LC/HC) exhibited stronger inhibition of HUVEC survival than 1121N (Ref B), which exhibited stronger inhibition of HUVEC survival than 34/86A (LC/HC), 34/86B (LC/HC), 29/88 (LC/HC), and V9/V9 (LC/HC). 34/86A (LC/HC), 34/86B (LC/HC), 29/99 (LC/HC), and V9/V9 (LC/HC) exhibited stronger inhibition of HUVEC survival than 1121B (Ref A), which exhibited stronger inhibition of HUVEC survival than 34/80A (LC/HC).

HUVEC tube formation assays were performed as described above using clones V9/V9 (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 1121B (Ref A), 1121N (Ref B), and Avastin. 1121N (Ref B) exhibited the strongest inhibition of HUVEC tube formation of all clones tested. 110A/110A (LC/HC) and 110B/110B (LC/HC) exhibited stronger inhibition of HUVEC tube formation than Avastin, which exhibited stronger inhibition of HUVEC tube formation than 29/88 (LC/HC). 29/88 (LC/HC) exhibited stronger inhibition of HUVEC tube formation than 109/109 (LC/HC), which exhibited stronger inhibition of HUVEC tube formation than 34/80B (LC/HC) and 34/86A (LC/HC). 34/80B (LC/HC) and 34/86A (LC/HC) exhibited stronger inhibition of HUVEC tube formation than V9/V9 (LC/HC), which exhibited stronger inhibition of HUVEC tube formation 34/86B (LC/HC).

HUVEC sprouting assays were performed as described above using clones V9/V9 (LC/HC), 34/80B (LC/HC), 34/86A (LC/HC), 34/86B (LC/HC), 29/88 (LC/HC), 109/109 (LC/HC), 110A/110A (LC/HC), 110B/110B (LC/HC), 1121B (Ref A), 1121N (Ref B), and Avastin. 1121N (Ref B) exhibited the strongest inhibition of HUVEC sprouting of all clones tested. 110A/110A (LC/HC) and 110B/110B (LC/HC) exhibited stronger inhibition of HUVEC sprouting than 109/109 (LC/HC), which exhibited stronger inhibition of HUVEC sprouting than 34/86A (LC/HC). 34/86A (LC/HC) exhibited stronger inhibition of HUVEC sprouting than 34/86B (LC/HC), which exhibited stronger inhibition of HUVEC sprouting than 29/88 (LC/HC). 29/88 (LC/HC) exhibited stronger inhibition of HUVEC sprouting than 34/80B (LC/HC), which exhibited stronger inhibition of HUVEC sprouting V9/V9 (LC/HC).

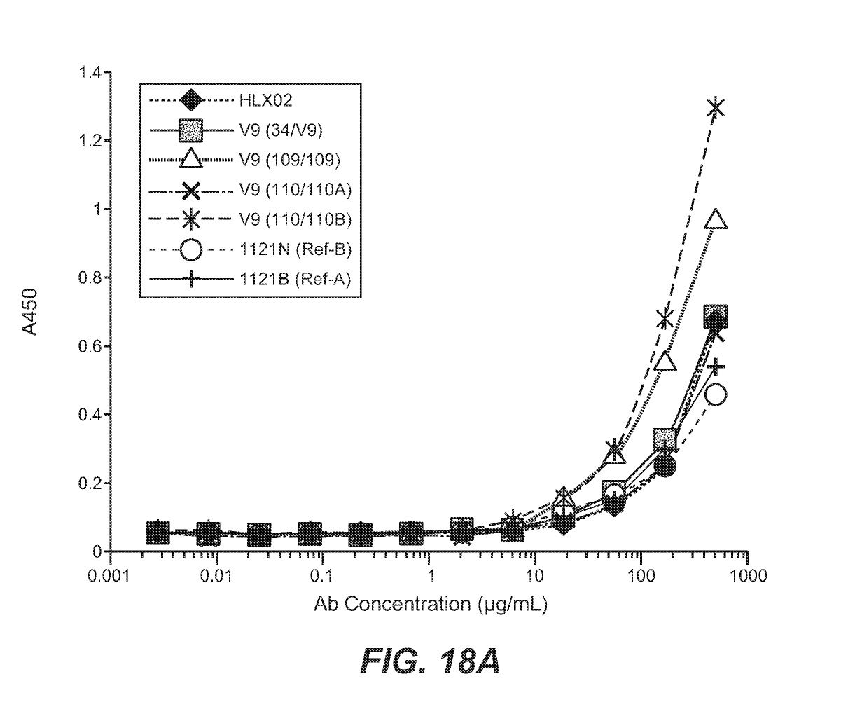

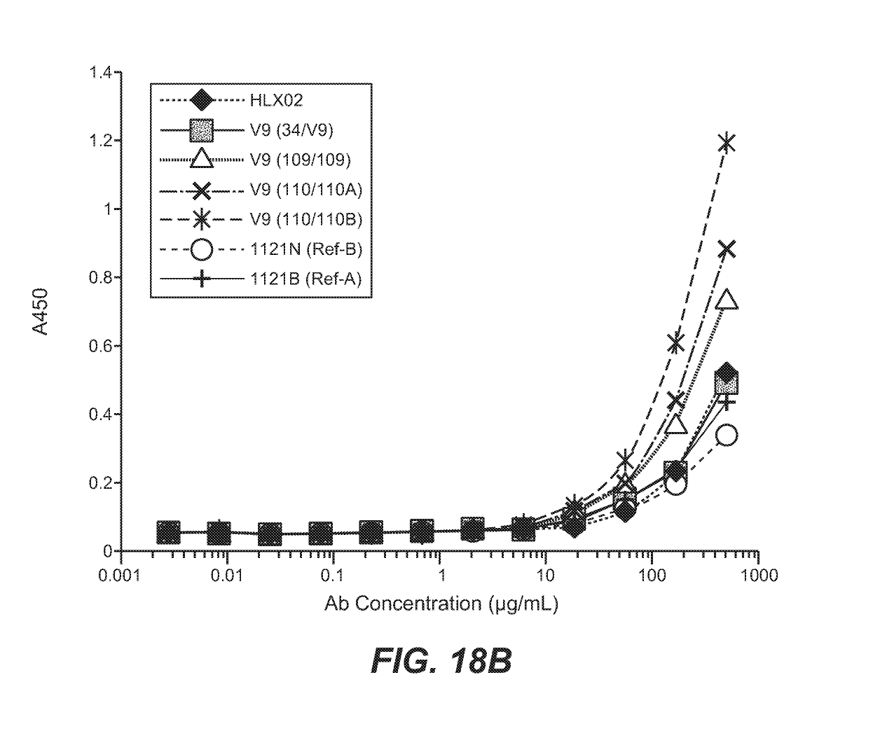

Another set of ELISAs was performed to assess the cross-species activity of clones 34/V9 (LC/HC), 109/109 (LC/HC), 110A/110A (LC/HC), and 110B/110B (LC/HC) as compared to 1121B (Ref A), 1121N (Ref B). Mouse VEGFR2-Fc was used as the capture reagent, and the amount of captured antibody in each well was quantified using an anti-Human Kappa-HRP-conjugated secondary antibody. The HRP-conjugated secondary antibody was added to the wells, and, following an incubation, excess secondary antibody was washed away. TMB was added to the wells, and following incubation, the reaction was stopped, and HRP activity was measured by monitoring the increase in absorbance at 450 nm. FIGS. 18A and 18B show the results of duplicate ELISA experiments, in which 110B/110B shows the strongest binding affinity for mouse VEGFR2.

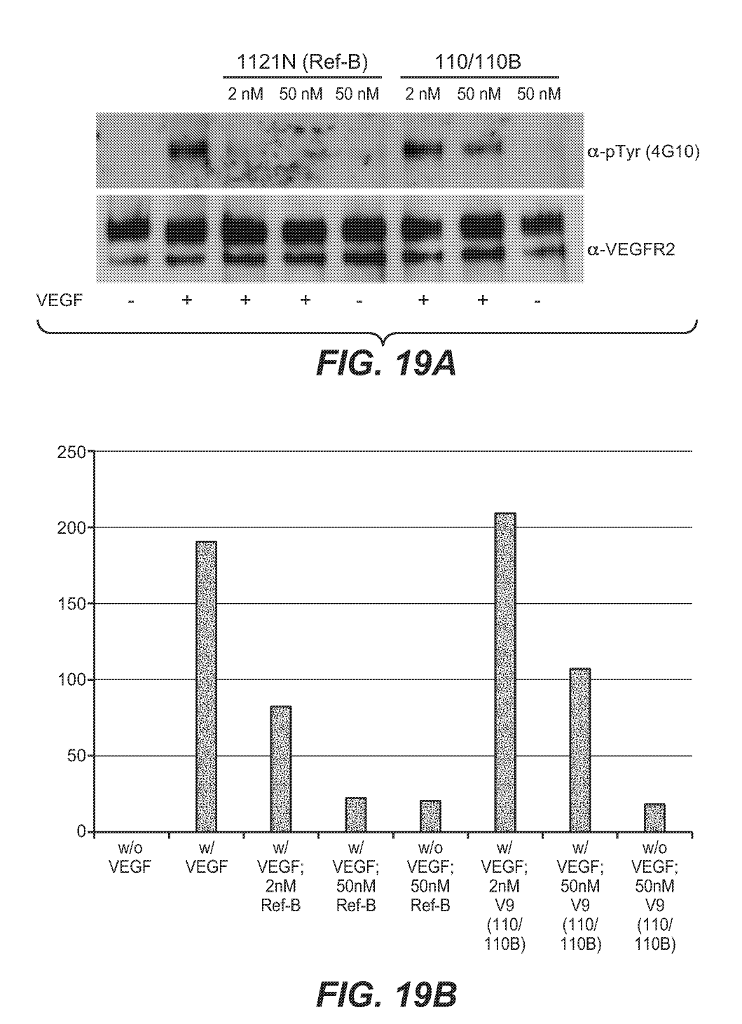

Next, assays were performed to assess the ability of 110/110B (LC/HC) to inhibit VEGFR2 phosphorylation as compared to 1121N (Ref B). Briefly, 8×10⁵ HUVEC cells were seeded in 10 cm dishes and grown to confluence. The media was then replaced with EBM-2 for 37° C. for serum starvation. Antibody was added to each dish and incubated for 30 minutes at 37° C. Next, 25 μg/ml of VEGF165 (eBioscience, Catalog #68-8784-82) was added to each dish for 10 minutes at 37° C. to stimulate VEGFR2. Following the addition of VEGF165, the media was aspirated, and the cells were washed with cold PBS. The PBS was discarded, and cell lysis buffer (Cell Signaling, Catalog #9803S)+1× Halt protease and phosphatase inhibitor cocktail (Pierce, Catalog #PIE78440)+5 mM Na₃VO₄ was added to each dish. After a 5 minute incubation at 4° C., the cells were collected and lysed, subject to two freeze-thaw cycles, and centrifuged at 14,000×g for 15 minutes at 4° C. The supernatants were transferred into new tubes and the total protein in each tube was quantified using BCA reagent (Pierce, Catalog #PIE23225). 150 μg of each total lysate was used in an immunoprecipitation reaction with 5 μg IMC-112N (i.e., Ref B, prepared in-house, lot #1405090516). The immunoprecipitation reactions were incubated overnight at 4° C. with tilt rotation. The immunoprecipitation reactions were then purified using protein A beads, washed, and analyzed via Western using a mouse anti-phosphotyrosine (4G10) antibody (Millipore, Catalog #05-1050) followed by an HRP-conjugated secondary antibody. ECL was added, and anti-phosphotyrosine antibody binding was detected by x-ray film. The membranes were then stripped using stripping buffer (Thermo, Catalog #21059) and probed with a rabbit anti-VEGFR2 (55B11) antibody (Cell Signaling, Catalog #2479) followed by an HRP-conjugated secondary antibody. ECL was added, and anti-VEGFR2 antibody binding was detected by x-ray film. As shown in FIG. 19A, 1121N (Ref B) inhibited VEGFR2 phosphorylation more strongly at lower concentrations than 110/110B (LC/HC). The results of FIG. 19A are quantified in FIG. 19B.

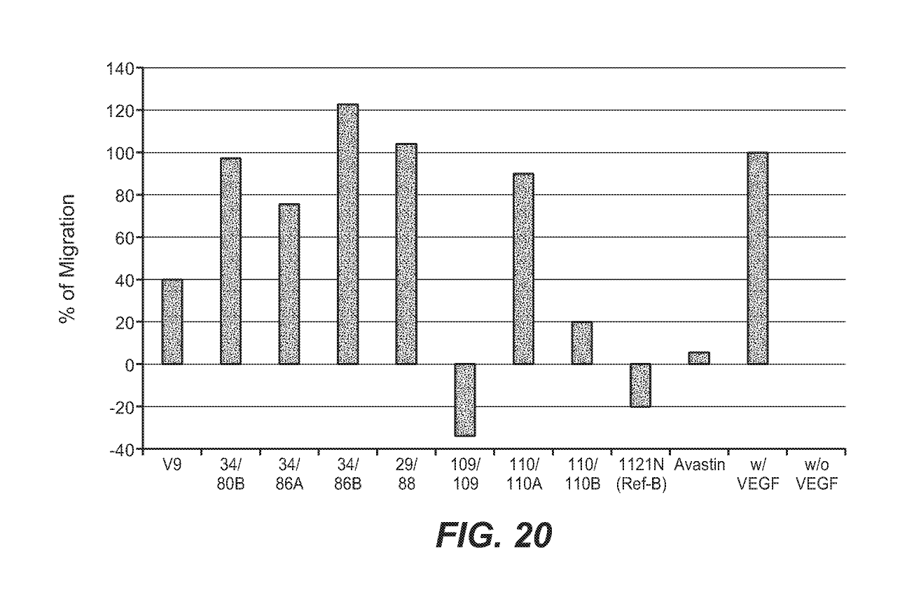

Migration assays as described above were performed to assess the abilities of clones V9, 34/80A (LC/HC), 34/86A (LC/HC), 34/86B (LC/HC), 29/88 (LC/HC), 109/109 (LC/HC), 110/110A (LC/HC), and 110/110B (LC/HC) to inhibit the migration activity of HUVECs. As shown in FIG. 20, 109/109 (LC/HC) demonstrated stronger inhibition of HUVEC migration than 1211N (Ref B).

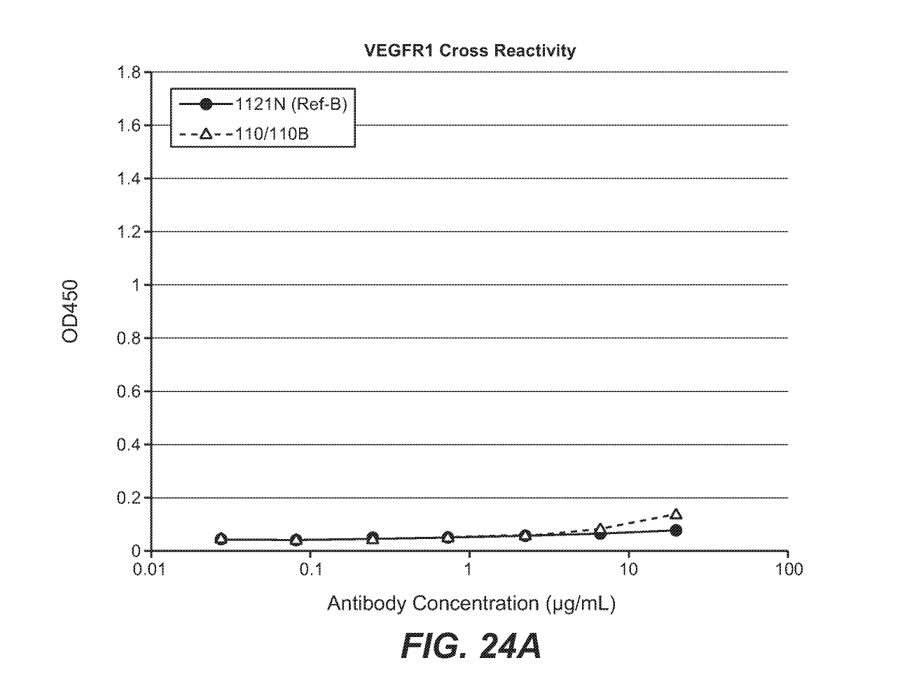

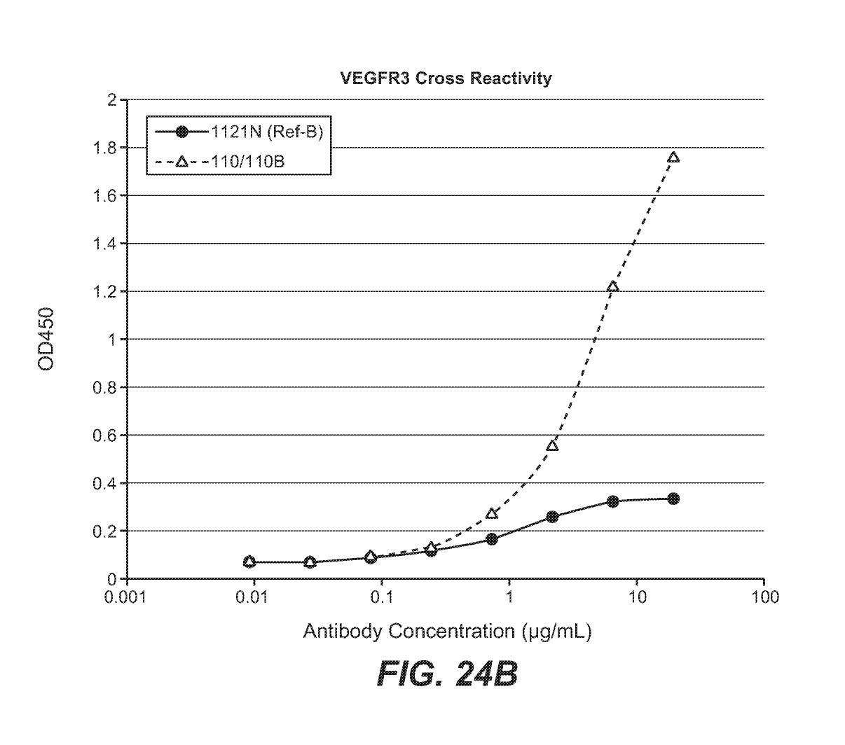

Next, ELISA experiments were performed to determine whether antibody 110/110B is capable of binding other members of the VEGFR2 family. Briefly, recombinant human VEGFR1-Fc fusion protein (R&D Systems) was immobilized in the wells of a 96-well plate. Antibody 1121N (Ref-B), antibody 110/110B, and mouse anti-human VEGFR1 (3× dilution from 10 μg/ml) were added to the wells. To detect binding of antibody to the antibodies to the immobilized VEGFR1-Fc, either goat anti-human F(ab)′₂-HRP or goat anti-mouse F(ab)′₂-HRP was added to each well. TMB was added to the wells, and following incubation at room temperature for 5 minutes, the reactions were stopped, and HRP activity was measured by monitoring the increase in absorbance at 450 nm. As shown in FIG. 24A, neither 110/110B nor 1121N (Ref-B) was found to bind VEGFR1-Fc.

In a similar set of ELISAs, 1121N (Ref-B), 110/110B, and mouse anti-human VEGFR3 (3× dilution from 10 μg/ml) were added to the wells of 96-well plates in which VEGFR3-Fc (R&D Systems) was immobilized. To detect binding of antibody to the antibodies to the immobilized VEGFR3-Fc, either goat anti-human F(ab)′₂-HRP or goat anti-mouse F(ab)′₂-HRP was added to each well. TMB was added to the wells, and following incubation at room temperature for 15 minutes, the reactions were stopped, and HRP activity was measured by monitoring the increase in absorbance at 450 nm. As shown in FIG. 24B, 110/110B was found to have better binding activity to human VEGFR3-Fc than 1121N (Ref-B).

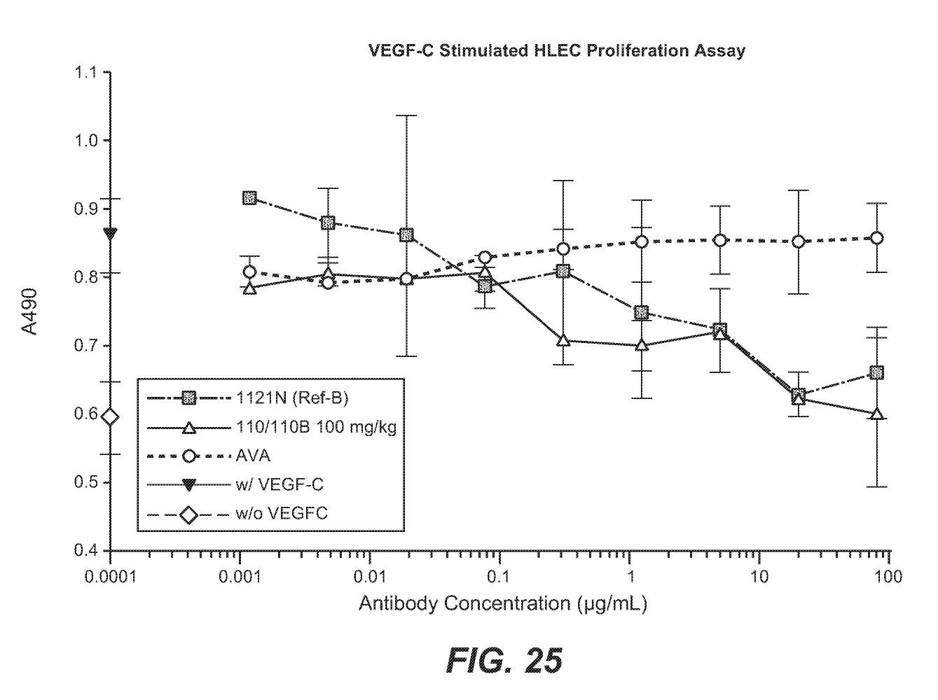

One of the main functions of VEGF-C is in lymphangiogenesis, wherein it acts on lymphatic endothelial cells (LECs) via VEGFR-3 to promote LEC survival, growth, and migration. Experiments were performed to determine the effect of antibody 110/110B on VEGF-C-induced human lymphatic endothelial cell (HLEC) proliferation. HLECs (ScienCell; P5) were detatched from plates, collected via centrifugation, and resuspended at 3×10⁴ cells/ml in endothelial cell medium (ECM) (ScienCell; Cat #1001). 100 μl of cell suspension was seeded into each well of a 96-well plate (i.e., to achieve a count of 3000 cells/well). After about 24 hours, the HLECs were washed once with PBS, and 70 μl of basal endothelial cell medium (ECM-b) (ScienCell; Cat #1001-b) was added to each well. 50 μl of ECM-b containing either 1121 (Ref-B), 110/110B, Avastin® was added to wells in 4× serial dilutions (i.e., from 240 μg/ml, which achieved a final concentration of 80 μg/ml/well) and incubated at 37° C. for 30 minutes. 30 μl of 2.5 μg/ml VEGF-C (PeproTech, Cat #100-20C) was added to half the wells (i.e., to achieve a final concentration of 500 ng/well), and the plate was incubated at 37° C. with 5% CO₂ for about 3 days (72 hours). 30 μl of MTS/PMS mixture (MTS (tetrazolium compound): Promega, Cat #G1112; PMS (electron coupling reagent): GeneLabs, Cat #AC-A2212.0005) was added to each well, and the plate was incubated at 37 C for 3.5 hours. The conversion of MTS into formazan is accomplished by dehydrogenase enzymes found in metabolically active cells. The quantity of formazan product as measured by the amount of 490 nm absorbance is directly proportional to the number of living cells in culture. As shown in FIG. 25, both 1121N (Ref-B) and 110/11B inhibit VEGF-C induced HELC proliferation.

Next, the effect of antibody 110/110B on VEGF-C-stimulated VEGFR2 phosphorylation was determined as follows: 1×10⁶ HLECs (ScienCell: P5) were seeded onto 10 cm dishes and grown to 80-90% confluence in endothelial cell medium (ECM) (ScienCell; Cat #1001). The medium was then replaced with ECM-b (ScienCell; Cat #1001-b) and incubated at 37° C. overnight. Antibody 1121N (Ref-B) or 110/110B were added to two sets of dishes. In one set of dishes, 0.288 μg/ml antibody was added to achieve a final concentration of 50 mM, and in the second set of dishes, 7.2 μg/ml antibody was added to achieve a final concentration of 2 mM. 200 mg/ml of VEGF-C (PeproTech; Cat #100-210C) was added and the dishes were incubated at 10 minutes at 37° C. to stimulate VEGFR2 phosphorylation. Following the incubation, the medium was aspirated, and each dish was washed once with cold 1×PBS. After the PBS was discarded, 300-400 μl of cell lysis buffer (CellSignaling; Cat #9803S) was added to each dish, followed by 1×HALT protease and phosphatase inhibitor cocktail (Pierce; Cat #PIE78440) and 5 mM Na₃VO₄. Following a 5 minute incubation at 4° C.; each cells lysate from each dish was collected and homogenized through a 27G needle. The homogenized cell lysates were transferred to tubes, subject to two freeze-thaw cycles on dry ice, and then centrifuged at 14,000×g for 15 minutes at 4° C. The supernatants were transferred to new tubes, and the total protein concentration in each tube was measured by BCA reagent (Pierce; Cat #PIE23225).

150 μg of each lysate was used in immunoprecipitation experiments, which were performed as follows: 5 μg antibody 1121N (Ref B) was added to each lysate and incubated with tilt rotation at 4° C. overnight. Next, Resin A was added to each antibody/lysate mixture and incubated with tilt rotation at 4° C. for 2 hours. Each of the mixtures were then added to Pierce Micro-A Pin columns (Pierce; Cat #89879), and the columns were centrifuged at 2000×g for 1 minute. The columns were then washed 5 times with 500 μl washing buffer (0.02% PBST+5 mM Na₃VO₄). 10-15 μl 5× sample buffer was added to each Resin A sample, and the mixtures were boiled at 95° C. for 5 minutes. Each sample was spun to pellet the Resin A, and each sample was resolved on a 6% SDS-PAGE gel. The protein was then transferred onto a PVDF membrane using standard methods. Following transfer, the membrane was blocked with 5% BSA/PBS.

First, the membrane was probed with 1000× diluted mouse anti-phosphotyrosine (4G10) in 1% BSA/0.05 PBST (Millipore: Cat #05-1050) and washed three times in 0.1% PBST for 5 minutes. Next, the membrane was probed with 1000× goat anti-mouse IgG (H+L) in 0.05% PBST at room temperature for 1 hour and washed three times in 0.1% PBST for 5 minutes. ECL was added, and anti-phosphotyrosine antibody binding was detected by x-ray film.

The membrane was striped with stripping buffer (Thermo; Cat#21059) at room temperature for 15 minutes and washed with 0.05% PBST for 5 minutes. The membrane was then blocked again with 5% BSA/PBS.

Next, the membrane was probed with 1000× diluted rabbit anti-VEGFR2 (55BB1) in 1% BSA/0.05 PBST (CellSignaling; Cat #2479) and washed three times in 0.1% PBST for 5 minutes. Next, the membrane was probed with 1000× goat anti-rabbit IgG (H+L) in 0.05% PBST at room temperature for 1 hour and washed three times in 0.1% PBST for 5 minutes. ECL was added, and anti-phosphotyrosine antibody binding was detected by x-ray film.

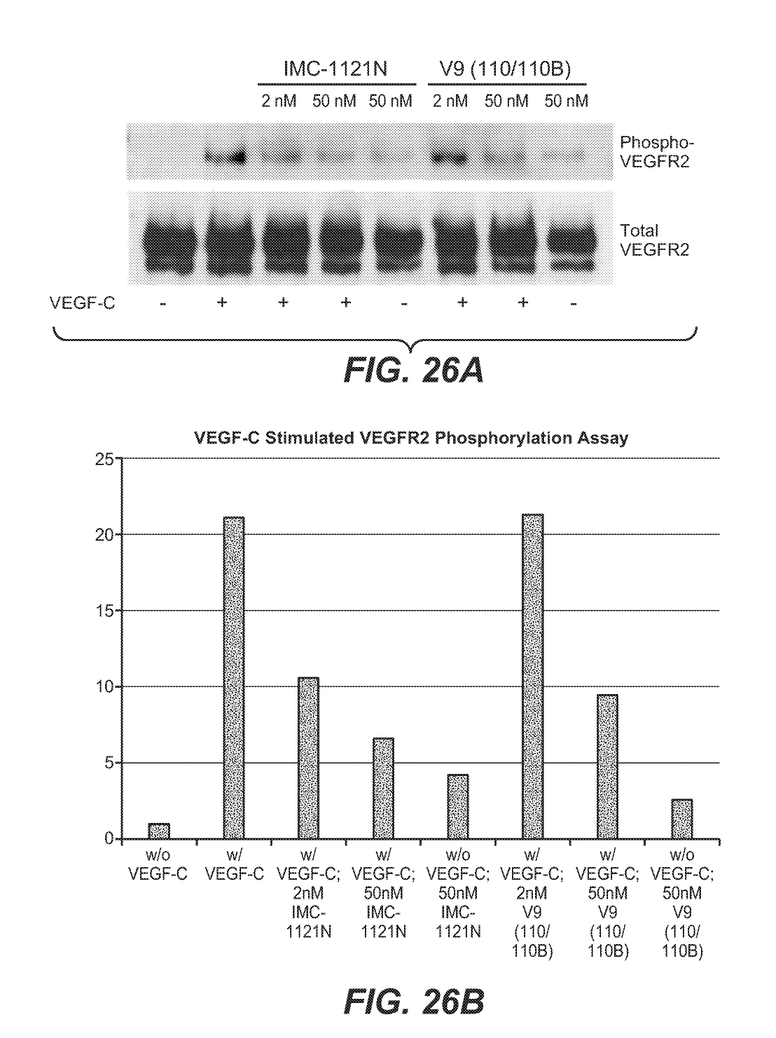

As shown in FIG. 26A, both 1121N (Ref B) and 110/110B could inhibit VEGF-C stimulated VEGFR2 phosphorylation. The quantitated results of FIG. 26A are shown in FIG. 26B. Antibody 1121N (Ref B) inhibited VEGFR2 phosphorylation more strongly than antibody 110/110B.

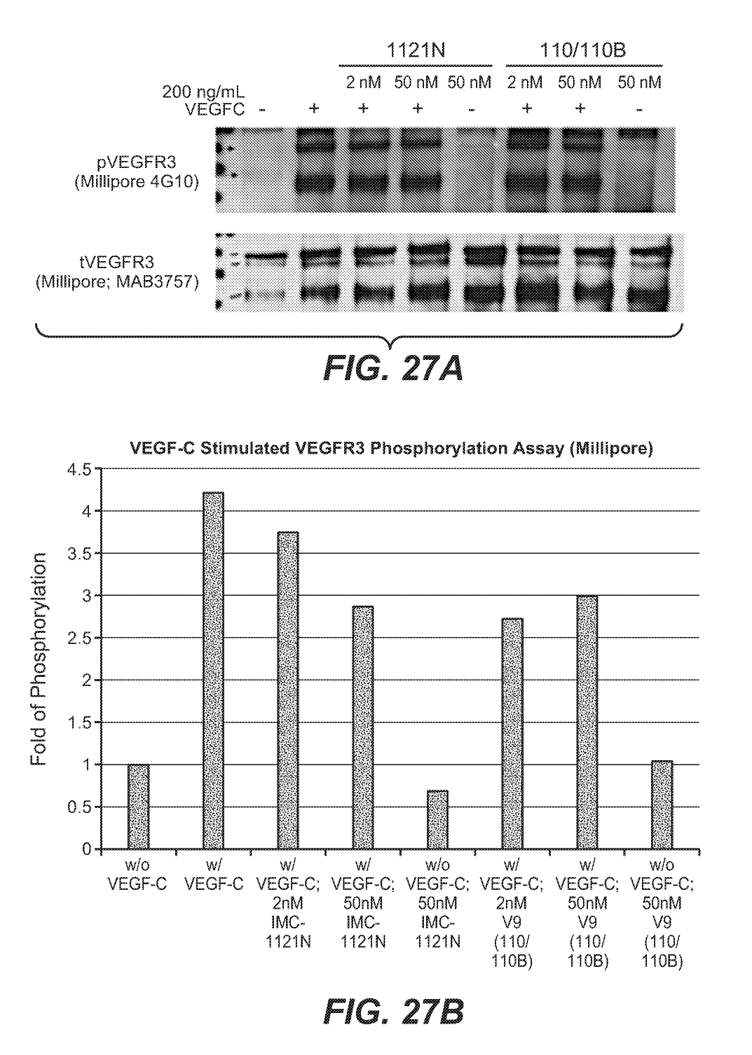

Similar experiments were performed to determine the effect of antibody 110/110B on VEGF-C-stimulated VEGFR3 phosphorylation. The experiments were performed as described above, using 1 μl goat anti-human VEGFR3 (R&D; Cat #AF349) or 3 μl mouse anti-VEGF3 (Millipore; Cat #MAB3757) instead of 5 μg antibody 1121N (Ref B) in the immunoprecipitation; and using protein G and protein A beads to precipitate the antibody/VEGFR3 complexes; and using 1000× diluted mouse anti-VEGFR3 (Millipore; Cat #MAB3575) instead of mouse anti-VEGFR2 to probe phosphorylated VEGF3. As shown in FIG. 27A, both antibody 1121N (Ref B) and antibody 110/110B could inhibit VEGF-C stimulated VEGFR3 phosphorylation. In FIG. 27B, which shows the quantified results of FIG. 27A, lower concentrations of 110/110B were shown to have a better inhibitory effect on VEGFR3 phosphorylation than lower concentrations of 1121N (Ref B).

Mice bearing human HCT-116 human colon cancer tumor xenografts were used to assay the therapeutic efficacy of clones 34/V9 (LC/HC), 109/109 (LC/HC), 110A/110A (LC/HC), and 110B/110B (LC/HC), as compared to 1121B (i.e., Ref A) 1121N (i.e., Ref B) and placebo. Briefly, human HCT-116 human colon cancer cells (inoculum=1×10⁶ cells) were implanted into female BALB/c nude mice. The mice were randomized into 7 groups. Each group was treated with one of the dosing regimens described in Table 11 below:

TABLE 11

| | | | Treatment | | |
|---|---|---|---|---|---|
| | | | Agent (Ab) | | Treatment |
| Group | Animals | Tumor | (LC/HC) | Dose | Schedule |
| 1 | BALB/c NU | HCT-116 | 34/V9 | 100 mg/kg | Day 3 after |
| 2 | Female | Inoculum = | 109/109 | | inoculation; |
| 3 | 6-7 weeks old | 1 × 10⁶ cells | 110A/110A | | twice a week |
| 4 | 4 mice/group | | 110B/110B | | for 3-4 weeks |
| 5 | | | 1121B (Ref A) | | |
| 6 | | | 1121N (Ref B) | | |
| 7 | | | Placebo | 10 ml/kg | |

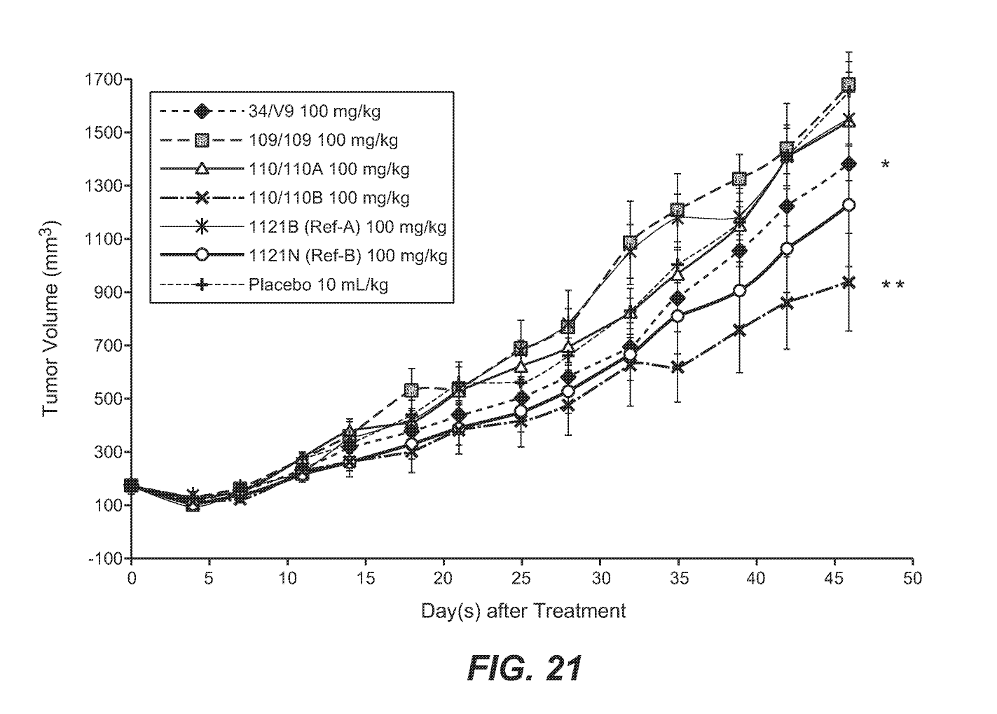

46 days following the beginning of treatment, mice treated with 110B/110B (100 mg/kg) demonstrated the greatest reduction in tumor burden as compared to 34/V9 (LC/HC) 109/109 (LC/HC), 110A/110A (LC/HC), 121B (i.e., Ref A), 1121N (i.e., Ref B), and placebo. Mice treated with 110B/110B (100 mg/kg) demonstrated greater reduction in tumor burden than mice treated with 109/109 (LC/HC), 110A/110A (LC/HC), 121B (i.e., Ref A), and placebo. See FIG. 21 and Table 12 below.

TABLE 12

| | TV¹ | RTV² | TV/CV %³ | TGI %⁴ | p Value of TV⁵ | |
|---|---|---|---|---|---|---|
| | (46 Days) | (46 Days) | (46 Days) | (46 Days) | (46 Days) | |
| 34/V9 | 1382 | 905% | 84% | 18% | 3 × 10⁻² | p < 0.05* |
| (100 mg/kg) | | | | | | |
| 109/109 | 1680 | 981% | 102% | −2% | 4 × 10⁻¹ | p > 0.05 |
| (100 mg/kg) | | | | | | |
| 110A/110A | 1538 | 908% | 93% | 8% | 3 × 10⁻¹ | p > 0.05 |
| (100 mg/kg) | | | | | | |
| 110B/110B | 933 | 579% | 56% | 48% | 3 × 10⁻³ | p < 0.01** |
| (100 mg/kg) | | | | | | |
| 1121B (Ref A) | 1553 | 912% | 94% | 7% | 3 × 10⁻¹ | p > 0.05 |
| (100 mg/kg) | | | | | | |
| 1121N (Ref B) | 1223 | 787% | 74% | 29% | 6 × 10⁻² | p > 0.05 |
| (100 mg/kg) | | | | | | |
| Placebo | 1654 | 989% | — | — | — | — |

¹TV: Tumor volume
²RTV: Tumor volume relative to initial
³TV/CV %: Treatment group volume/Control group volume
⁴TGI %: Tumor growth inhibition rate = 1 − (T46 − T0)/(C46 − C0)%
⁵p value: <0.05 = * <0.01 = ** <0.001 = ***

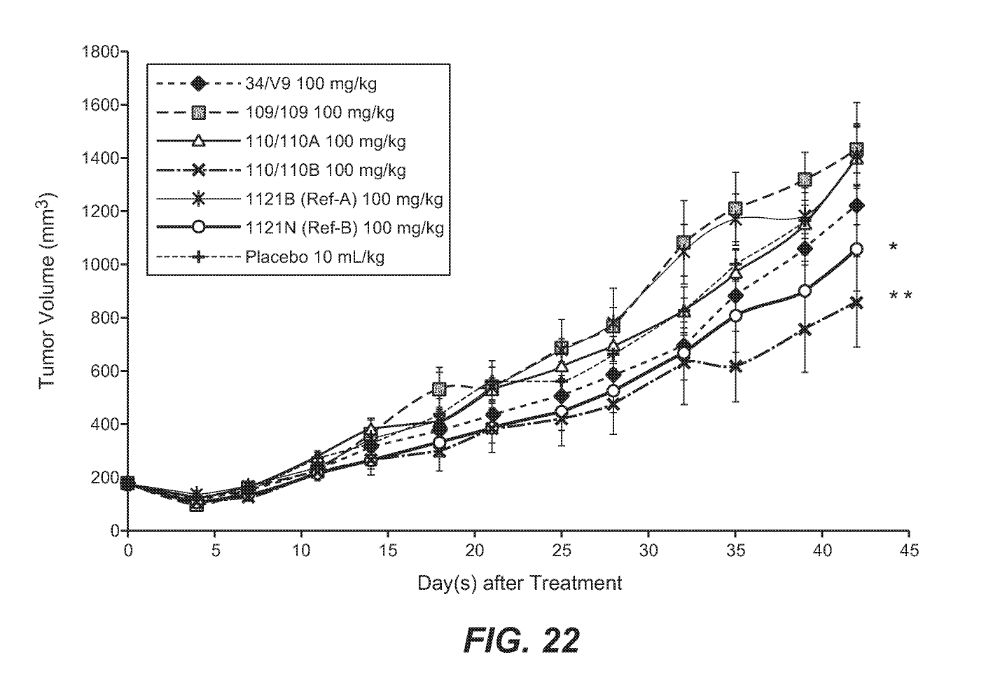

Additional data from the xenograft experiments described in Table 11 are provided herein. As shown in FIG. 22 and Table 13 below, 42 days following the beginning of treatment, mice treated with 110B/110B (100 mg/kg) demonstrated the greatest reduction in tumor burden as compared to 34/V9 (LC/HC) 109/109 (LC/HC), 110A/110A (LC/HC), 121B (i.e., Ref A), 1121N (i.e., Ref B), and placebo. Mice treated with 110B/110B (100 mg/kg) demonstrated greater reduction in tumor burden than mice treated with 109/109 (LC/HC), 110A/110A (LC/HC), 121B (i.e., Ref A), and placebo.

TABLE 13

| | TV¹ | RTV² | TV/CV %³ | TGI %⁴ | p Value of TV⁵ | |
|---|---|---|---|---|---|---|
| | (42 Days) | (42 Days) | (42 Days) | (42 Days) | (42 Days) | |
| 34/V9 | 1221 | 803% | 87% | 15% | 9 × 10⁻² | p > 0.05 |
| (100 mg/kg) | | | | | | |
| 109/109 | 1435 | 833% | 102% | −2% | 4 × 10⁻¹ | p > 0.05 |
| (100 mg/kg) | | | | | | |
| 110A/110A | 1403 | 832% | 100% | 0% | 5 × 10⁻¹ | p > 0.05 |
| (100 mg/kg) | | | | | | |
| 110B/110B | 858 | 525% | 61% | 44% | 8 × 10⁻³ | p < 0.01** |
| (100 mg/kg) | | | | | | |
| 1121B (Ref A) | 1404 | 838% | 100% | 0% | 5 × 10⁻¹ | p > 0.05 |
| (100 mg/kg) | | | | | | |
| 1121N (Ref B) | 1055 | 660% | 75% | 29% | 4 × 10⁻² | p < 0.05* |
| (100 mg/kg) | | | | | | |
| Placebo | 1407 | 841% | — | — | | — |

¹TV: Tumor volume
²RTV: Tumor volume relative to initial
³TV/CV %: Treatment group volume/Control group volume
⁴TGI %: Tumor growth inhibition rate = 1 − (T42 − T0)/(C42 − C0)%
⁵p value: <0.05 = * <0.01 = ** <0.001 = ***

Four formulations of antibody 34/V9 and antibody 110/110B (i.e., F1-F4, as described below in Table 14) were prepared at the concentrations listed in Table 15 and tested stored at either −80° C. or 40° C. for two weeks.

TABLE 14

| | BUFFER | EXCIPIENTS | PH |
|---|---|---|---|
| F1 | 10 mM Histidine | 75 mM NaCl, 1% Glycine, | 6 |
| | | 2% Sucrose, 0.02% Tween 80 | |
| F2 | 10 mM Citrate | 75 mM NaCl, 2% Glycine, | 6 |
| | | 2% Sucrose, 0.02% Tween 80 | |
| F3 | 10 mM Acetate | 75 mM NaCl, 2% Glycine, | 6 |
| | | 2% Sucrose, 0.02% Tween 80 | |
| F4 | 10 mM Phosphate | 75 mM NaCl, 1% Glycine, | 6 |
| | | 2% Sucrose, 0.02% Tween 80 | |

TABLE 15

| | | Concentration | Storage |
|---|---|---|---|
| Antibody | Formulation | (μg/ml) | Temperature |
| 34/V9 (LC/HC) | F1 | 1974 | −80° C. |
| | F1 | 1527 | 40° C. |
| | F2 | 2441 | −80° C. |
| | F2 | 2677 | 40° C. |
| | F3 | 1358 | −80° C. |
| | F3 | 1669 | 40° C. |
| | F4 | 2033 | −80° C. |
| | F4 | 2225 | 40° C. |
| 110/110B (LC/HC) | F1 | 1734 | −80° C. |
| | F1 | 1797 | 40° C. |
| | F2 | 2172 | −80° C. |
| | F2 | 2773 | 40° C. |
| | F3 | 2150 | −80° C. |
| | F3 | 2306 | 40° C. |
| | F4 | 2124 | −80° C. |
| | F4 | 2381 | 40° C. |

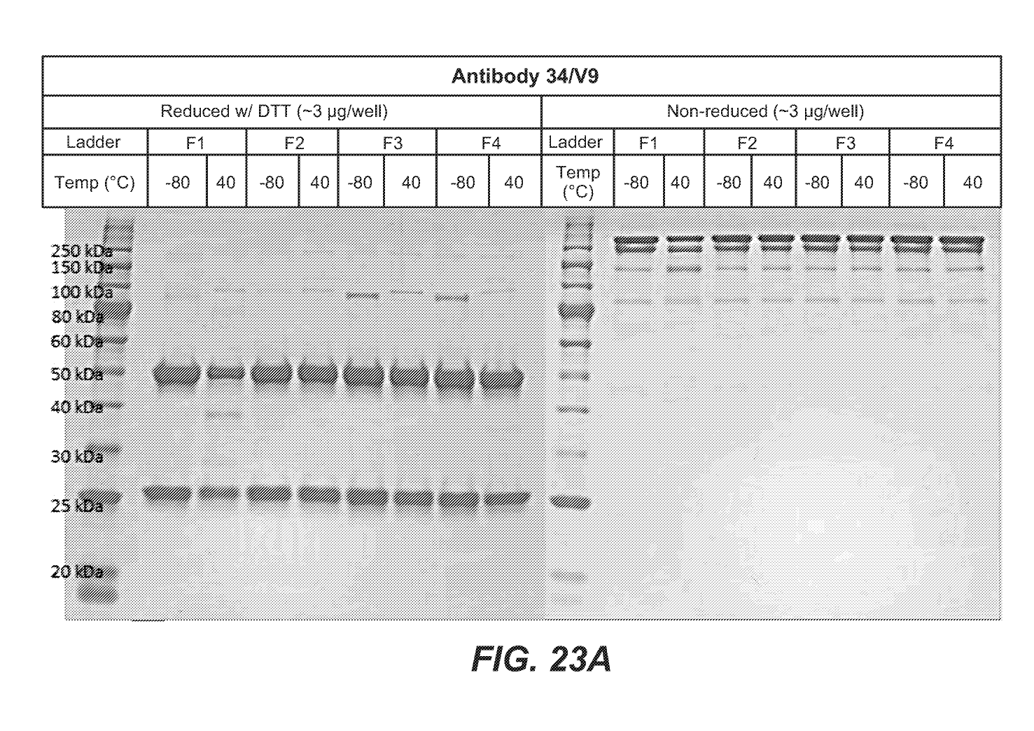

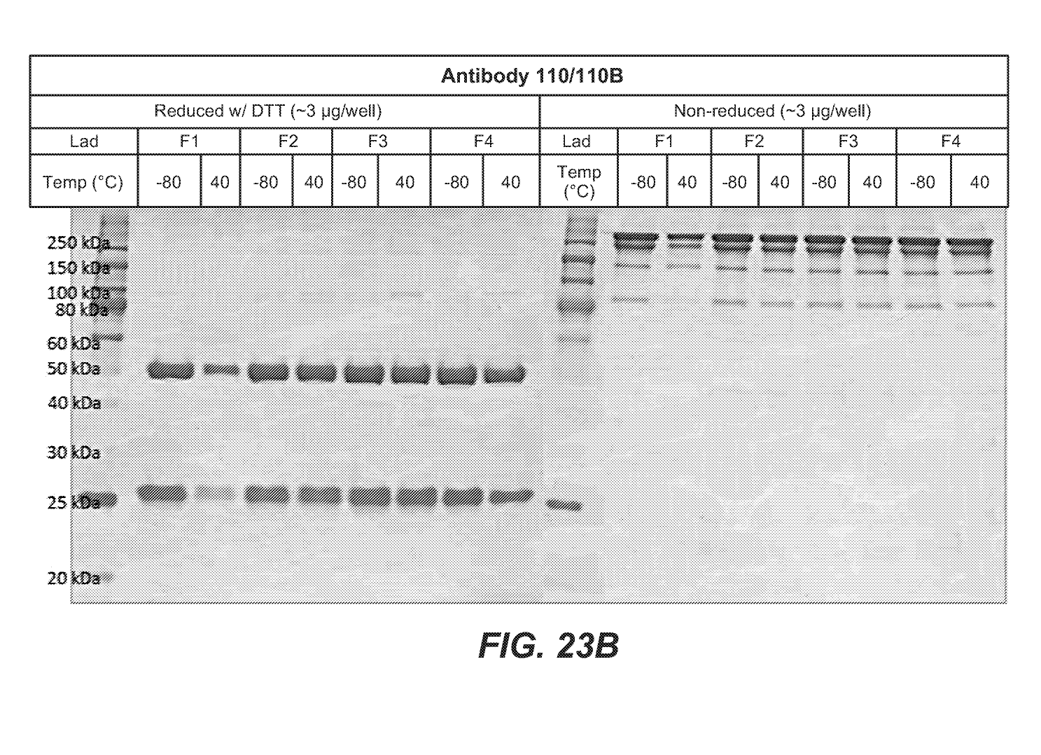

Samples of antibody from each formulation under each storage condition were then analyzed via electrophoresis under both reducing and non-reducing conditions. As shown in FIG. 23A, the stability of antibody 34/V9 was highest the F2 formulation. The stability of 34/V9 was approximately equivalent in F3 and F4, and 34/V9 was the least stable in F1. The same results were seen for antibody 110/110B. See FIG. 23B.

Mice bearing human NCI-H460 non-small cell lung cancer (NSCLC) tumor xenografts were used to assay the therapeutic efficacy of 110B/110B, as compared to placebo. Briefly, human NCI-H460 NSCLC cells (inoculum=3×10⁶ cells) were implanted into female BALB/c nude mice. The mice were randomized into 4 groups. Each group was treated with one of the dosing regimens described in Table 16 below:

TABLE 16

| | | | Treatment | | |
|---|---|---|---|---|---|
| | | | Agent (Ab) | | Treatment |
| Group | Animals | Tumor | (LC/HC) | Dose | Schedule |
| 1 | BALB/c NU | NCI-H460 | 110B/110B | 100 mg/kg | Day 4 after |
| 2 | Female | Inoculum = | | 50 mg/kg | inoculation; |
| 3 | ~6-7 weeks | 3 × 10⁶ cells | | 25 mg/kg | twice a week |
| 4 | old | | Placebo | 10 ml/kg | for 3 weeks |
| | ~3-4 mice/ | | | | |
| | group | | | | |

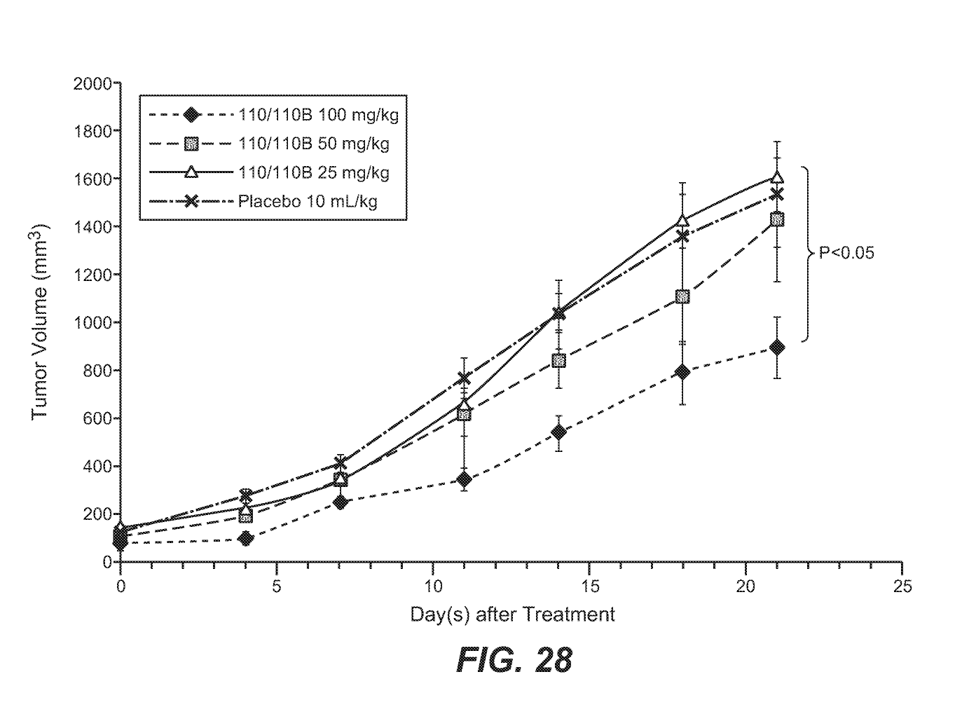

21 days following the beginning of treatment, mice treated with 110/110B (100 mg/kg) demonstrated the greatest reduction in tumor burden as compared to mice treated with 110/110B (50 mg/kg), 110/110B (25 mg/kg), and placebo. See FIG. 28 and Table 17 below.

TABLE 17

| | TV¹ | RTV² | TV/ | TGI %⁴ | | |
|---|---|---|---|---|---|---|
| | (21 | (21 | CV %³ | (21 | p Value of TV⁵ | |
| | Days) | Days) | (21 Days) | Days) | (21 Days) | |
| 100/100B | 893 | 1181% | 58% | 42% | 0.015 | p > 0.05 |
| (100 mg/kg) | | | | | | |
| 100/100B | 1425 | 1406% | 93% | 6% | 0.392 | p > 0.05 |
| (50 mg/kg) | | | | | | |
| 100/100B | 1606 | 1121% | 105% | −4% | 0.397 | 0.392 |
| (25 mg/kg) | | | | | | |
| Placebo | 1529 | 1302% | — | — | | — |

¹TV: Tumor volume
²RTV: Tumor volume relative to initial
³TV/CV %: Treatment group volume/Control group volume
⁴TGI %: Tumor growth inhibition rate = 1 − (T42 − T0)/(C42 − C0)%
⁵p value: <0.05 = * <0.01 = ** <0.001 = ***

The preceding Examples are offered for illustrative purposes only, and are not intended to limit the scope of the present invention in any way. Various modifications of the invention in addition to those shown and described herein will become apparent to those skilled in the art from the foregoing description and fall within the scope of the appended claims.

### LIST OF EMBODIMENTS

1. An anti-VEGFR2 antibody or antigen binding fragment thereof, wherein the antibody or antigen binding fragment thereof does not block the binding of VEGFR2 to VEGF.

2. An anti-VEGFR2 antibody or antigen binding fragment thereof, wherein the antibody or antigen binding fragment thereof binds domains 5-7 of VEGFR2.

3. An anti-VEGFR2 antibody or antigen binding fragment thereof, wherein the antibody or antigen binding fragment thereof does not block the binding of VEGFR2 to VEGF, and wherein the antibody or antigen binding fragment thereof binds domains 5-7 of VEGFR2.

4. The anti-VEGFR2 antibody or antigen binding fragment thereof of any one of embodiments 1-3, wherein the antibody binds to whole HUVEC cells.

5. The anti-VEGFR2 antibody or antigen binding fragment thereof of any one of embodiments 1-4, wherein the antibody or antigen binding fragment thereof does not inhibit angiogenesis in vitro.

6. The anti-VEGFR2 antibody or antigen binding fragment thereof according to any one of embodiments 1-4, wherein the antibody or antigen binding fragment thereof inhibits angiogenesis in vivo.

7. The anti-VEGFR2 antibody or antigen binding fragment thereof according to any one of embodiments 1-4, wherein the antibody or antigen binding fragment thereof does not inhibit angiogenesis in vitro, and wherein the antibody or antigen binding fragment thereof inhibits angiogenesis in vivo.

8. An anti-vascular endothelial growth factor receptor 2 (VEGFR2) antibody or antigen binding fragment thereof comprising a light chain variable domain sequence comprising (1) a CDR-L1 comprising the amino acid sequence RASQNIASYLN (SEQ ID NO: 76) or RASQSVS-S/N-S/N-YL-G/A (SEQ ID NO: 83) or TRSRGSIASSYVQ (SEQ ID NO: 80) or RSSQSL-L/V/Y-H/Y-G/S/R-D/N-G-N/K/Y-N/T-Y/F-LD (SEQ ID NO: 84); (2) a CDR-L2 comprising the amino acid sequence L/A/G/K/E-G/A/V/N/S-S/D-N/S/Q/K-R/L-A/K/D/P-S/T (SEQ ID NO: 60); and (3) a CDR-L3 comprising the amino acid sequence M/Q-Q/S-A/S/R/G/Y-L/Y/S/A/D/G/T-Q/S/N/H/F-T/I/W/S-P/T-Y/L/P/V/G/I-T/V (SEQ ID NO: 72); and a heavy chain variable domain sequence comprising (1) a CDR-H1 comprising the amino acid sequence T/S-Y-Y/G/A/S-M/I-H/N/S (SEQ ID NO: 34); (2) a CDR-H2 comprising the amino acid sequence I/V/G/S-I-N/S/I-P/Y/S/G-S/D/I-G/F/S-G/S-S/N/T/Y/A-T/K/A/I-S/Y/N/H-YA-Q/D-K/S-F/V-K/Q-G (SEQ ID NO: 40); and (3) a CDR-H3 comprising the amino acid sequence GLWFGEGY (SEQ ID NO: 49) or ESYGGQFDY (SEQ ID NO: 43) or DLVVPAATLDY (SEQ ID NO:42) or D/G-F/I-Y/I-E/V-A/G-G/P-G/T-W/D-Y/A-FD-L/I (SEQ ID NO: 51) or RDGSLGVGYYYMDF (SEQ ID NO: 50) or VGATTSLYYYYGMDV (SEQ ID NO: 47) or DGFGLAVAGPYWYFDL (SEQ ID NO: 44) or PTRSRDFWSGLGYYYYMDV (SEQ ID NO: 45).

9. The anti-VEGFR2 antibody or antigen binding fragment thereof of embodiment 8, wherein the light chain variable domain sequence comprises (1) a CDR-L1 comprising an amino acid sequence selected from the group consisting of SEQ ID NOs: 75-82; (2) a CDR-L2 comprising an amino acid sequence selected from the group consisting of SEQ ID NOs: 54-59; and (3) a CDR-L3 comprising an amino acid sequence selected from the group consisting of SEQ ID NOs: 63-71; and a heavy chain variable domain sequence comprising (1) a CDR-H1 comprising an amino acid sequence selected from the group consisting of SEQ ID NOs: 29-33 and 85; (2) a CDR-H2 comprising an amino acid sequence selected from the group consisting of SEQ ID NOs: 35-39 and 125; and (3) a CDR-H3 comprising an amino acid sequence selected from the group consisting of SEQ ID NOs: 42-50.

10. The anti-VEGFR2 antibody or antigen binding fragment thereof of embodiment 1 or 2, wherein the light chain variable domain sequence comprises (1) a CDR-L1 comprising the amino acid sequence RSSQSLLHGNGNNYLD (SEQ ID NO: 75); (2) a CDR-L2 comprising the amino acid sequence LGSNRAS (SEQ ID NO: 54); and (3) a CDR-L3 comprising the amino acid sequence MQALQTPYT (SEQ ID NO: 63); and a heavy chain variable domain sequence comprising (1) a CDR-H1 comprising the amino acid sequence TYYMH (SEQ ID NO: 29); (2) a CDR-H2 comprising the amino acid sequence IINPSGGSTSYAQKFQG (SEQ ID NO: 36); and (3) a CDR-H3 comprising the amino acid sequence DLVVPAATLDY (SEQ ID NO: 42).

11. The anti-VEGFR2 antibody or antigen binding fragment thereof of embodiment 8 or 9, wherein the light chain variable domain sequence comprises (1) a CDR-L1 comprising the amino acid sequence RASQNIASYLN (SEQ ID NO: 76); (2) a CDR-L2 comprising the amino acid sequence AASSLKS (SEQ ID NO: 55); and (3) a CDR-L3 comprising the amino acid sequence QQSYSIPYT (SEQ ID NO: 64); and a heavy chain variable domain sequence comprising (1) a CDR-H1 comprising the amino acid sequence SYGMH (SEQ ID NO: 30); (2) a CDR-H2 comprising the amino acid sequence VISYDGSNKYYADSVKG (SEQ ID NO: 37); and (3) a CDR-H3 comprising the amino acid sequence ESYGGQFDY (SEQ ID NO: 43).

12. The anti-VEGFR2 antibody or antigen binding fragment thereof of embodiment 8 or 9, wherein the light chain variable domain sequence comprises (1) a CDR-L1 comprising the amino acid sequence RASQSVSNNYLG (SEQ ID NO: 77) (2) a CDR-L2 comprising the amino acid sequence GASSRAT (SEQ ID NO: 56); and (3) a CDR-L3 comprising the amino acid sequence QQRSNWPLT (SEQ ID NO: 65); and a heavy chain variable domain sequence comprising (1) a CDR-H1 comprising the amino acid sequence SYAMH (SEQ ID NO: 31); (2) a CDR-H2 comprising the amino acid sequence VISYDGSNKYYADSVKG (SEQ ID NO: 37); and (3) a CDR-H3 comprising the amino acid sequence DGFGLAVAGPYWYFDL (SEQ ID NO: 44).

13. The anti-VEGFR2 antibody or antigen binding fragment thereof of embodiment 8 or 9, wherein the light chain variable domain sequence comprises (1) a CDR-L1 comprising the amino acid sequence RSSQSLVYSDGKTYLD (SEQ ID NO: 78); (2) a CDR-L2 comprising the amino acid sequence KVSNRDS (SEQ ID NO: 57); and (3) a CDR-L3 comprising the amino acid sequence MQGAHWPPT (SEQ ID NO: 66); and a heavy chain variable domain sequence comprising (1) a CDR-H1 comprising the amino acid sequence SYAIS (SEQ ID NO: 85); (2) a CDR-H2 comprising the amino acid sequence GIIPIFGTANYAQKFQG (SEQ ID NO: 38); and (3) a CDR-H3 comprising the amino acid sequence PTRSRDFWSGLGYYYYMDV (SEQ ID NO: 45).

14. The anti-VEGFR2 antibody or antigen binding fragment thereof of embodiment 8 or 9, wherein the light chain variable domain sequence comprises (1) a CDR-L1 comprising the amino acid sequence RASQSVSSSYLA (SEQ ID NO: 79); (2) a CDR-L2 comprising the amino acid sequence set forth in GASSRAT (SEQ ID NO: 56); and (3) a CDR-L3 comprising the amino acid sequence QQRSNWPPT (SEQ ID NO: 67) and a heavy chain variable domain sequence comprising (1) a CDR-H1 comprising the amino acid sequence SYGMH (SEQ ID NO: 30); (2) a CDR-H2 comprising the amino acid sequence set forth in VISYDGSNKHYADSVKG (SEQ ID NO: 125); and (3) a CDR-H3 comprising the amino acid sequence set forth in DFYEAGGWYFDL (SEQ ID NO: 46).

15. The anti-VEGFR2 antibody or antigen binding fragment thereof of embodiment 8 or 9, wherein the light chain variable domain sequence comprises (1) a CDR-L1 comprising the amino acid sequence TRSRGSIASSYVQ (SEQ ID NO: 80); (2) a CDR-L2 comprising the amino acid sequence ENDQRPS (SEQ ID NO: 58); and (3) a CDR-L3 comprising the amino acid sequence QSYDFSTVV (SEQ ID NO: 68); and a heavy chain variable domain sequence comprising (1) a CDR-H1 comprising the amino acid sequence SYAIS (SEQ ID NO: 85); (2) a CDR-H2 comprising the amino acid sequence set forth in GIIPIFGTANYAQKFQG (SEQ ID NO: 38); and (3) a CDR-H3 comprising the amino acid sequence VGATTSLYYYYGMDV (SEQ ID NO: 47).

16. The anti-VEGFR2 antibody or antigen binding fragment thereof of embodiment 8 or 9, wherein the light chain variable domain sequence comprises (1) a CDR-L1 comprising the amino acid sequence RASQSVSSSYLA (SEQ ID NO: 79); (2) a CDR-L2 comprising the amino acid sequence GASSRAT (SEQ ID NO: 56); and (3) a CDR-L3 comprising the amino acid sequence QQYGSSPGT (SEQ ID NO: 69); and a heavy chain variable domain sequence comprising (1) a CDR-H1 comprising the amino acid sequence SYSMN (SEQ ID NO: 28); (2) a CDR-H2 comprising the amino acid sequence SISSSSSYIYYADSVKG (SEQ ID NO: 35); and (3) a CDR-H3 comprising the amino acid sequence GIIVGPTDAFDI (SEQ ID NO: 48).

17. The anti-VEGFR2 antibody or antigen binding fragment thereof of embodiment 8 or 9, wherein the light chain variable domain sequence comprises (1) a CDR-L1 comprising the amino acid sequence RSSQSLYYRDGYTFLD (SEQ ID NO: 81); (2) a CDR-L2 comprising the amino acid sequence LSSKRDS (SEQ ID NO: 59); and (3) a CDR-L3 comprising the amino acid sequence MQGTHWPYT (SEQ ID NO: 70); and a heavy chain variable domain sequence comprising (1) a CDR-H1 comprising the amino acid sequence TYAMS (SEQ ID NO: 33); (2) a CDR-H2 comprising the amino acid sequence GISGSGGATHYADSVKG (SEQ ID NO: 39); and (3) a CDR-H3 comprising the amino acid sequence GLWFGEGY (SEQ ID NO: 49).

18. The anti-VEGFR2 antibody or antigen binding fragment thereof of embodiment 8 or 9, wherein the light chain variable domain sequence comprises (1) a CDR-L1 comprising the amino acid sequence RSSQSLLYSNGYNYLD (SEQ ID NO: 82); (2) a CDR-L2 comprising the amino acid sequence LGSNRAS SEQ ID NO: 54); and (3) a CDR-L3 comprising the amino acid sequence MQALQTPIT (SEQ ID NO: 71); and a heavy chain variable domain sequence comprising (1) a CDR-H1 comprising the amino acid sequence SYAIS (SEQ ID NO: 85); (2) a CDR-H2 comprising the amino acid sequence GIIPIFGTANYAQKFQG (SEQ ID NO: 38); and (3) a CDR-H3 comprising the amino acid sequence RDGSLGVGYYYMDF (SEQ ID NO: 50).

19. An anti-vascular endothelial growth factor receptor 2 (VEGFR2) antibody or antigen binding fragment that is a variant of an anti-VEGFR2 antibody comprising a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27), wherein the variant comprises at least one amino acid substitution in one or more of SEQ ID NOs: 22, 23, 24, 25, 26, and/or 27.

20. An anti-vascular endothelial growth factor receptor 2 (VEGFR2) antibody or antigen binding fragment thereof that competitively inhibits the binding of a second anti-VEGFR2 antibody to VEGFR2, wherein the second anti-VEGFR2 antibody comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27).

21. An anti-vascular endothelial growth factor receptor 2 (VEGFR2) antibody or antigen binding fragment thereof that specifically binds to the same epitope of VEGFR2 as a second anti-VEGFR2 antibody to VEGFR2, wherein the second anti-VEGFR2 antibody comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27).

22. The anti-VEGFR2 antibody or antigen binding fragment of embodiment 20 or 21, wherein the antibody comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27), wherein the variant comprises at least one amino acid substitution in one or more of SEQ ID NOs: 22, 23, 24, 25, 26, and/or 27.

23. The anti-VEGFR2 antibody or antigen binding fragment thereof of any one of embodiments 20-22, wherein the antibody comprises a light chain variable domain sequence comprising (1) a CDR-L1 comprising an amino acid sequence selected from consisting of SEQ ID NOs: 1 and 16; (2) a CDR-L2 comprising an amino acid sequence selected from consisting of SEQ ID NOs: 2, 7, and 8; and (3) a CDR-L3 comprising an amino acid sequence selected from consisting of SEQ ID NOs: 3, 9, and 12; and a heavy chain variable domain sequence comprising (1) a CDR-H1 comprising an amino acid sequence selected from consisting of SEQ ID NOs: 4, 13, 14, and 15; (2) a CDR-H2 comprising an amino acid sequence selected from consisting of SEQ ID NOs: 5, 7, 17, 18, 19, 20, and 21; (3) a CDR-H3 comprising an amino acid sequence selected from consisting of SEQ ID NOs: 6, 10, and 11.

24. The anti-VEGFR2 antibody or antigen binding fragment thereof of any one of embodiments 20-22, wherein the light chain variable domain sequence comprises (1) a CDR-L1 comprising the amino acid sequence QSLYYRDGYTF (SEQ ID NO: 1); (2) a CDR-L2 comprising the amino acid sequence LSS (SEQ ID NO: 2); and (3) a CDR-L3 comprising the amino acid sequence MQGTHWPYT (SEQ ID NO: 3); and a heavy chain variable domain sequence comprises (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

25. The anti-VEGFR2 antibody or antigen binding fragment thereof of any one of embodiments 20-22, wherein the antibody comprises a light chain variable domain sequence comprising (1) a CDR-L1 comprising the amino acid sequence QSLYYRDGYTF (SEQ ID NO:1); (2) a CDR-L2 comprising the amino acid sequence QSS (SEQ ID NO: 7); and (3) a CDR-L3 comprising the amino acid sequence MQGTHWPYT (SEQ ID NO: 3); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

26. The anti-VEGFR2 antibody or antigen binding fragment thereof of any one of embodiments 20-22, wherein the antibody comprises a light chain variable domain sequence comprising a (1) CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence LQGTHWPYT (SEQ ID NO: 9); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGL (SEQ ID NO: 10).

27. The anti-VEGFR2 antibody or antigen binding fragment thereof of any one of embodiments 20-22, wherein the antibody comprises a light chain variable domain sequence comprising (1) a CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence FQGTHWPYT (SEQ ID NO: 12); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

28. The anti-VEGFR2 antibody or antigen binding fragment thereof of any one of embodiments 20-22, wherein the antibody comprises a light chain variable domain sequence comprising (1) a CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence LQGTHWPYT (SEQ ID NO: 9); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

29. The anti-VEGFR2 antibody or antigen binding fragment thereof of any one of embodiments 20-22, wherein the antibody comprises a light chain variable domain sequence comprising (1) a CDR-L1 comprising the amino acid sequence QSLYYRSGYTF (SEQ ID NO: 16); (2) a CDR-L2 comprising the amino acid sequence QSS (SEQ ID NO: 7); and (3) a CDR-L3 comprising the amino acid sequence MQGTHWPYT (SEQ ID NO: 3); and a heavy chain variable domain sequence comprising (1) a CDR-H1 comprising the amino acid sequence RFSFSTYA (SEQ ID NO: 15); (2) a CDR-H2 comprising the amino acid sequence ISGSGQAT (SEQ ID NO: 20); and (3) a CDR-H3 comprising the amino acid sequence KGLWFGEGY (SEQ ID NO: 6).

30. The anti-VEGFR2 antibody or antigen binding fragment thereof of any one of embodiments 20-22, wherein the antibody comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGTT (SEQ ID NO: 21); and a CDR-H3 comprising the amino acid sequence KGLWFGEGY (SEQ ID NO: 6).

31. The anti-VEGFR2 antibody or antigen binding fragment thereof of any one of embodiments 20-22, wherein the antibody comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGTT (SEQ ID NO: 21); and a CDR-H3 comprising the amino acid sequence KGLWFGEGL (SEQ ID NO: 10).

32. The anti-VEGFR2 antibody or antigen binding fragment thereof according to any one of embodiments 1-31, wherein the antibody comprises an Fc sequence of a human IgG.

33. The antigen binding fragment of the anti-VEGFR2 antibody according to any one of embodiments 1-32, wherein the antigen binding fragment is selected from the group consisting of a Fab, Fab′, a F(ab)′ 2, a single-chain Fv (scFv), an Fv fragment, a diabody, and a linear antibody.

34. The anti-VEGFR2 antibody of any one of embodiments 1-33, wherein the antibody is a multi-specific antibody.

35. The anti-VEGFR2 antibody or antigen binding fragment thereof according to any one of embodiments 1-34 conjugated to a therapeutic agent.

36. The anti-VEGFR2 antibody or antigen binding fragment thereof according to any one of embodiments 1-34 conjugated to a label.

37. The anti-VEGFR2 antibody according to embodiment 36, wherein the label is selected from the group consisting of a radioisotope, a fluorescent dye, and an enzyme.

38. An isolated nucleic acid molecule that encodes the anti-VEGFR2 antibody or antigen binding fragment thereof according to any one of embodiments 1-34.

39. An expression vector encoding the nucleic acid molecule of embodiment 38.

40. A cell comprising the expression vector of embodiment 39.

41. A method of producing an anti-VEGFR2 comprising culturing the cell of embodiment 40 and recovering the anti-VEGFR2 from the cell culture.

42. A composition comprising the anti-VEGFR2 antibody or antigen binding fragment thereof according to any one of embodiments 1-35 and a pharmaceutically acceptable carrier.

43. A method of detecting a VEGFR2 protein in sample from a patient by contacting the anti-VEGFR2 antibody or antigen binding fragment thereof according to any one of embodiments 1-34 and 36-37 to the sample and detecting the anti-VEGFR2 antibody bound to the VEGFR2 protein.

44. The method according to embodiment 43, wherein the anti-VEGFR2 antibody or antigen binding fragment thereof is used an immunohistochemistry assay (IHC) or in an ELISA assay.

45. A method of treating pathological condition characterized by excessive angiogenesis in a subject, comprising administering an effective amount of the composition of embodiment 42 to the subject.

46. The method of embodiment 45, wherein the pathological condition characterized by excessive angiogenesis is selected from the group consisting of cancer, an ocular disease, or inflammation.

47. The method of embodiment 46, wherein the pathological condition characterized by excessive angiogenesis is cancer.

48. The method of embodiment 47, wherein the cancer is colon cancer, colorectal cancer, gastric cancer, gastroesophageal cancer, bladder cancer, lung cancer, or solid tumor.

49. The method of embodiment 48, wherein the cancer is lung cancer, and wherein the lung cancer is non-small cell lung cancer (NSCLC).

50. The method of embodiment 45-49, wherein the subject is further administered a therapeutic agent selected from the group consisting of an anti-neoplastic agent, a chemotherapeutic agent, a growth inhibitory agent and a cytotoxic agent.

What is claimed is:

1. An anti-VEGFR2 antibody or antigen binding fragment thereof, wherein the antibody or antigen binding fragment thereof does not block the binding of VEGFR2 to VEGF.

2. An anti-VEGFR2 antibody or antigen binding fragment thereof, wherein the antibody or antigen binding fragment thereof binds domains 5-7 of VEGFR2.

3. An anti-VEGFR2 antibody or antigen binding fragment thereof, wherein the antibody or antigen binding fragment thereof does not block the binding of VEGFR2 to VEGF, and wherein the antibody or antigen binding fragment thereof binds domains 5-7 of VEGFR2.

4. The anti-VEGFR2 antibody or antigen binding fragment thereof of any one of claims 1-3, wherein the antibody binds to whole HUVEC cells.

5. The anti-VEGFR2 antibody or antigen binding fragment thereof of any one of claims 1-4, wherein the antibody or antigen binding fragment thereof does not inhibit angiogenesis in vitro.

6. The anti-VEGFR2 antibody or antigen binding fragment thereof according to any one of claims 1-4, wherein the antibody or antigen binding fragment thereof inhibits angiogenesis in vivo.

7. The anti-VEGFR2 antibody or antigen binding fragment thereof according to any one of claims 1-4, wherein the antibody or antigen binding fragment thereof does not inhibit angiogenesis in vitro, and wherein the antibody or antigen binding fragment thereof inhibits angiogenesis in vivo.

8. An anti-vascular endothelial growth factor receptor 2 (VEGFR2) antibody or antigen binding fragment thereof comprising a light chain variable domain sequence comprising (1) a CDR-L1 comprising the amino acid sequence RASQNIASYLN (SEQ ID NO: 76) or RASQSVS-S/N-S/N-YL-G/A (SEQ ID NO: 83) or TRSRGSIASSYVQ (SEQ ID NO: 80) or RSSQSL-L/V/Y-H/Y-G/S/R-D/N-G-N/K/Y-N/T-Y/F-LD (SEQ ID NO: 84); (2) a CDR-L2 comprising the amino acid sequence L/A/G/K/E-G/A/V/N/S-S/D-N/S/Q/K-R/L-A/K/D/P-S/T (SEQ ID NO: 60); and (3) a CDR-L3 comprising the amino acid sequence M/Q-Q/S-A/S/R/G/Y-L/Y/S/A/D/G/T-Q/S/N/H/F-T/I/W/S-P/T-Y/L/P/V/G/I-T/V (SEQ ID NO: 72); and a heavy chain variable domain sequence comprising (1) a CDR-H1 comprising the amino acid sequence T/S-Y-Y/G/A/S-M/I-H/N/S (SEQ ID NO: 34); (2) a CDR-H2 comprising the amino acid sequence I/V/G/S-I-N/S/I-P/Y/S/G-S/D/I-G/F/S-G/S-S/N/T/Y/A-T/K/A/I-S/Y/N/H-YA-Q/D-K/S-F/V-K/Q-G (SEQ ID NO: 40); and (3) a CDR-H3 comprising the amino acid sequence GLWFGEGY (SEQ ID NO: 49) or ESYGGQFDY (SEQ ID NO: 43) or DLVVPAATLDY (SEQ ID NO:42) or D/G-F/I-Y/I-E/V-A/G-G/P-G/T-W/D-Y/A-FD-L/I (SEQ ID NO: 51) or RDGSLGVGYYYMDF (SEQ ID NO: 50) or VGATTSLYYYYGMDV (SEQ ID NO: 47) or DGFGLAVAGPYWYFDL (SEQ ID NO: 44) or PTRSRDFWSGLGYYYYMDV (SEQ ID NO: 45).

9. The anti-VEGFR2 antibody or antigen binding fragment thereof of claim 8, wherein the light chain variable domain sequence comprises (1) a CDR-L1 comprising an amino acid sequence selected from the group consisting of SEQ ID NOs: 75-82; (2) a CDR-L2 comprising an amino acid sequence selected from the group consisting of SEQ ID NOs: 54-59; and (3) a CDR-L3 comprising an amino acid sequence selected from the group consisting of SEQ ID NOs: 63-71; and a heavy chain variable domain sequence comprising (1) a CDR-H1 comprising an amino acid sequence selected from the group consisting of SEQ ID NOs: 29-33 and 85; (2) a CDR-H2 comprising an amino acid sequence selected from the group consisting of SEQ ID NOs: 35-39 and 125; and (3) a CDR-H3 comprising an amino acid sequence selected from the group consisting of SEQ ID NOs: 42-50.

10. The anti-VEGFR2 antibody or antigen binding fragment thereof of claim 1 or 2, wherein the light chain variable domain sequence comprises (1) a CDR-L1 comprising the amino acid sequence RSSQSLLHGNGNNYLD (SEQ ID NO: 75); (2) a CDR-L2 comprising the amino acid sequence LGSNRAS (SEQ ID NO: 54); and (3) a CDR-L3 comprising the amino acid sequence MQALQTPYT (SEQ ID NO: 63); and a heavy chain variable domain sequence comprising (1) a CDR-H1 comprising the amino acid sequence TYYMH (SEQ ID NO: 29); (2) a CDR-H2 comprising the amino acid sequence IINPSGGSTSYAQKFQG (SEQ ID NO: 36); and (3) a CDR-H3 comprising the amino acid sequence DLVVPAATLDY (SEQ ID NO: 42).

11. The anti-VEGFR2 antibody or antigen binding fragment thereof of claim 8 or 9, wherein the light chain variable domain sequence comprises (1) a CDR-L1 comprising the amino acid sequence RASQNIASYLN (SEQ ID NO: 76); (2) a CDR-L2 comprising the amino acid sequence AASSLKS (SEQ ID NO: 55); and (3) a CDR-L3 comprising the amino acid sequence QQSYSIPYT (SEQ ID NO: 64); and a heavy chain variable domain sequence comprising (1) a CDR-H1 comprising the amino acid sequence SYGMH (SEQ ID NO: 30); (2) a CDR-H2 comprising the amino acid sequence VISYDGSNKYYADSVKG (SEQ ID NO: 37); and (3) a CDR-H3 comprising the amino acid sequence ESYGGQFDY (SEQ ID NO: 43).

12. The anti-VEGFR2 antibody or antigen binding fragment thereof of claim 8 or 9, wherein the light chain variable domain sequence comprises (1) a CDR-L1 comprising the amino acid sequence RASQSVSNNYLG (SEQ ID NO: 77) (2) a CDR-L2 comprising the amino acid sequence GASSRAT (SEQ ID NO: 56); and (3) a CDR-L3 comprising the amino acid sequence QQRSNWPLT (SEQ ID NO: 65); and a heavy chain variable domain sequence comprising (1) a CDR-H1 comprising the amino acid sequence SYAMH (SEQ ID NO: 31); (2) a CDR-H2 comprising the amino acid sequence VISYDGSNKYYADSVKG (SEQ ID NO: 37); and (3) a CDR-H3 comprising the amino acid sequence DGFGLAVAGPYWYFDL (SEQ ID NO: 44).

13. The anti-VEGFR2 antibody or antigen binding fragment thereof of claim 8 or 9, wherein the light chain variable domain sequence comprises (1) a CDR-L1 comprising the amino acid sequence RSSQSLVYSDGKTYLD (SEQ ID NO: 78); (2) a CDR-L2 comprising the amino acid sequence KVSNRDS (SEQ ID NO: 57); and (3) a CDR-L3 comprising the amino acid sequence MQGAHWPPT (SEQ ID NO: 66); and a heavy chain variable domain sequence comprising (1) a CDR-H1 comprising the amino acid sequence SYAIS (SEQ ID NO: 85); (2) a CDR-H2 comprising the amino acid sequence GIIPIFGTANYAQKFQG (SEQ ID NO: 38); and (3) a CDR-H3 comprising the amino acid sequence PTRSRDFWSGLGYYYYMDV (SEQ ID NO: 45).

14. The anti-VEGFR2 antibody or antigen binding fragment thereof of claim 8 or 9, wherein the light chain variable domain sequence comprises (1) a CDR-L1 comprising the amino acid sequence RASQSVSSSYLA (SEQ ID NO: 79); (2) a CDR-L2 comprising the amino acid sequence set forth in GASSRAT (SEQ ID NO: 56); and (3) a CDR-L3 comprising the amino acid sequence QQRSNWPPT (SEQ ID NO: 67) and a heavy chain variable domain sequence comprising (1) a CDR-H1 comprising the amino acid sequence SYGMH (SEQ ID NO: 30); (2) a CDR-H2 comprising the amino acid sequence set forth in VISYDGSNKHYADSVKG (SEQ ID NO: 125); and (3) a CDR-H3 comprising the amino acid sequence set forth in DFYEAGGWYFDL (SEQ ID NO: 46).

15. The anti-VEGFR2 antibody or antigen binding fragment thereof of claim 8 or 9, wherein the light chain variable domain sequence comprises (1) a CDR-L1 comprising the amino acid sequence TRSRGSIASSYVQ (SEQ ID NO: 80); (2) a CDR-L2 comprising the amino acid sequence ENDQRPS (SEQ ID NO: 58); and (3) a CDR-L3 comprising the amino acid sequence QSYDFSTVV (SEQ ID NO: 68); and a heavy chain variable domain sequence comprising (1) a CDR-H1 comprising the amino acid sequence SYAIS (SEQ ID NO: 85); (2) a CDR-H2 comprising the amino acid sequence set forth in GIIPIFGTANYAQKFQG (SEQ ID NO: 38); and (3) a CDR-H3 comprising the amino acid sequence VGATTSLYYYYGMDV (SEQ ID NO: 47).

16. The anti-VEGFR2 antibody or antigen binding fragment thereof of claim 8 or 9, wherein the light chain variable domain sequence comprises (1) a CDR-L1 comprising the amino acid sequence RASQSVSSSYLA (SEQ ID NO: 79); (2) a CDR-L2 comprising the amino acid sequence GASSRAT (SEQ ID NO: 56); and (3) a CDR-L3 comprising the amino acid sequence QQYGSSPGT (SEQ ID NO: 69); and a heavy chain variable domain sequence comprising (1) a CDR-H1 comprising the amino acid sequence SYSMN (SEQ ID NO: 28); (2) a CDR-H2 comprising the amino acid sequence SISSSSSYIYYADSVKG (SEQ ID NO: 35); and (3) a CDR-H3 comprising the amino acid sequence GIIVGPTDAFDI (SEQ ID NO: 48).

17. The anti-VEGFR2 antibody or antigen binding fragment thereof of claim 8 or 9, wherein the light chain variable domain sequence comprises (1) a CDR-L1 comprising the amino acid sequence RSSQSLYYRDGYTFLD (SEQ ID NO: 81); (2) a CDR-L2 comprising the amino acid sequence LSSKRDS (SEQ ID NO: 59); and (3) a CDR-L3 comprising the amino acid sequence MQGTHWPYT (SEQ ID NO: 70); and a heavy chain variable domain sequence comprising (1) a CDR-H1 comprising the amino acid sequence TYAMS (SEQ ID NO: 33); (2) a CDR-H2 comprising the amino acid sequence GISGSGGATHYADSVKG (SEQ ID NO: 39); and (3) a CDR-H3 comprising the amino acid sequence GLWFGEGY (SEQ ID NO: 49).

18. The anti-VEGFR2 antibody or antigen binding fragment thereof of claim 8 or 9, wherein the light chain variable domain sequence comprises (1) a CDR-L1 comprising the amino acid sequence RSSQSLLYSNGYNYLD (SEQ ID NO: 82); (2) a CDR-L2 comprising the amino acid sequence LGSNRAS SEQ ID NO: 54); and (3) a CDR-L3 comprising the amino acid sequence MQALQTPIT (SEQ ID NO: 71); and a heavy chain variable domain sequence comprising (1) a CDR-H1 comprising the amino acid sequence SYAIS (SEQ ID NO: 85); (2) a CDR-H2 comprising the amino acid sequence GIIPIFGTANYAQKFQG (SEQ ID NO: 38); and (3) a CDR-H3 comprising the amino acid sequence RDGSLGVGYYYMDF (SEQ ID NO: 50).

19. An anti-vascular endothelial growth factor receptor 2 (VEGFR2) antibody or antigen binding fragment that is a variant of an anti-VEGFR2 antibody comprising a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27), wherein the variant comprises at least one amino acid substitution in one or more of SEQ ID NOs: 22, 23, 24, 25, 26, and/or 27.

20. An anti-vascular endothelial growth factor receptor 2 (VEGFR2) antibody or antigen binding fragment thereof that competitively inhibits the binding of a second anti-VEGFR2 antibody to VEGFR2, wherein the second anti-VEGFR2 antibody comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27).

21. An anti-vascular endothelial growth factor receptor 2 (VEGFR2) antibody or antigen binding fragment thereof that specifically binds to the same epitope of VEGFR2 as a second anti-VEGFR2 antibody to VEGFR2, wherein the second anti-VEGFR2 antibody comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27).

22. The anti-VEGFR2 antibody or antigen binding fragment of claim 20 or 21, wherein the antibody comprises a light chain variable domain sequence comprising (1) a CDR-L1 comprising the amino acid sequence QSLYYR-D/S-GYTF (SEQ ID NO: 22); (2) a CDR-L2 comprising the amino acid sequence L/Q/R-SS (SEQ ID NO: 23); and (3) a CDR-L3 comprising the amino acid sequence M/L/F-QGTHWPYT (SEQ ID NO: 24); and a heavy chain variable domain sequence comprising (1) a CDR-H1 comprising the amino acid sequence G/R-F-S/T/P-FSTYA (SEQ ID NO: 25); (2) a CDR-H2 comprising the amino acid sequence I-S/N-G-S/N-G/S-G/Q-A/T-T (SEQ ID NO: 26); and (3) a CDR-H3 comprising the amino acid sequence KGLWFGEG-Y/L/I (SEQ ID NO: 27), wherein the variant comprises at least one amino acid substitution in one or more of SEQ ID NOs: 22, 23, 24, 25, 26, and/or 27.

23. The anti-VEGFR2 antibody or antigen binding fragment thereof of any one of claims 20-22, wherein the antibody comprises a light chain variable domain sequence comprising (1) a CDR-L1 comprising an amino acid sequence selected from consisting of SEQ ID NOs: 1 and 16; (2) a CDR-L2 comprising an amino acid sequence selected from consisting of SEQ ID NOs: 2, 7, and 8; and (3) a CDR-L3 comprising an amino acid sequence selected from consisting of SEQ ID NOs: 3, 9, and 12; and a heavy chain variable domain sequence comprising (1) a CDR-H1 comprising an amino acid sequence selected from consisting of SEQ ID NOs: 4, 13, 14, and 15; (2) a CDR-H2 comprising an amino acid sequence selected from consisting of SEQ ID NOs: 5, 7, 17, 18, 19, 20, and 21; (3) a CDR-H3 comprising an amino acid sequence selected from consisting of SEQ ID NOs: 6, 10, and 11.

24. The anti-VEGFR2 antibody or antigen binding fragment thereof of any one of claims 20-22, wherein the light chain variable domain sequence comprises (1) a CDR-L1 comprising the amino acid sequence QSLYYRDGYTF (SEQ ID NO: 1); (2) a CDR-L2 comprising the amino acid sequence LSS (SEQ ID NO: 2); and (3) a CDR-L3 comprising the amino acid sequence MQGTHWPYT (SEQ ID NO: 3); and a heavy chain variable domain sequence comprises (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

25. The anti-VEGFR2 antibody or antigen binding fragment thereof of any one of claims 20-22, wherein the antibody comprises a light chain variable domain sequence comprising (1) a CDR-L1 comprising the amino acid sequence QSLYYRDGYTF (SEQ ID NO:1); (2) a CDR-L2 comprising the amino acid sequence QSS (SEQ ID NO: 7); and (3) a CDR-L3 comprising the amino acid sequence MQGTHWPYT (SEQ ID NO: 3); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

26. The anti-VEGFR2 antibody or antigen binding fragment thereof of any one of claims 20-22, wherein the antibody comprises a light chain variable domain sequence comprising a (1) CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence LQGTHWPYT (SEQ ID NO: 9); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGL (SEQ ID NO: 10).

27. The anti-VEGFR2 antibody or antigen binding fragment thereof of any one of claims 20-22, wherein the antibody comprises a light chain variable domain sequence comprising (1) a CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence FQGTHWPYT (SEQ ID NO: 12); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

28. The anti-VEGFR2 antibody or antigen binding fragment thereof of any one of claims 20-22, wherein the antibody comprises a light chain variable domain sequence comprising (1) a CDR-L1 comprising the amino acid sequence QSLYYRDGYTF (SEQ ID NO: 1); (2) a CDR-L2 comprising the amino acid sequence QSS (SEQ ID NO: 7); and (3) a CDR-L3 comprising the amino acid sequence LQGTHWPYT (SEQ ID NO: 9); and a heavy chain variable domain sequence comprising (1) a CDR-H1 comprising the amino acid sequence GFSFSTYA (SEQ ID NO: 4); (2) a CDR-H2 comprising the amino acid sequence ISGSGGAT (SEQ ID NO: 5); and (3) a CDR-H3 comprising the amino acid sequence KGLWFGEGY (SEQ ID NO: 6).

29. The anti-VEGFR2 antibody or antigen binding fragment thereof of any one of claims 20-22, wherein the antibody comprises a light chain variable domain sequence comprising (1) a CDR-L1 comprising the amino acid sequence QSLYYRSGYTF (SEQ ID NO: 16); (2) a CDR-L2 comprising the amino acid sequence QSS (SEQ ID NO: 7); and (3) a CDR-L3 comprising the amino acid sequence MQGTHWPYT (SEQ ID NO: 3); and a heavy chain variable domain sequence comprising (1) a CDR-H1 comprising the amino acid sequence RFSFSTYA (SEQ ID NO: 15); (2) a CDR-H2 comprising the amino acid sequence ISGSGQAT (SEQ ID NO: 20); and (3) a CDR-H3 comprising the amino acid sequence KGLWFGEGY (SEQ ID NO: 6).

30. The anti-VEGFR2 antibody or antigen binding fragment thereof of any one of claims 20-22, wherein the antibody comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGTT (SEQ ID NO: 21); and a CDR-H3 comprising the amino acid sequence KGLWFGEGY (SEQ ID NO: 6).

31. The anti-VEGFR2 antibody or antigen binding fragment thereof of any one of claims 20-22, wherein the antibody comprises a light chain variable domain sequence comprising a CDR-L1 comprising the amino acid sequence QSLYYRSGYTF (SEQ ID NO: 16); a CDR-L2 comprising the amino acid sequence QSS (SEQ ID NO: 7); and a CDR-L3 comprising the amino acid sequence FQGTHWPYT (SEQ ID NO: 12); and a heavy chain variable domain sequence comprising a CDR-H1 comprising the amino acid sequence GFSFSTYA (SEQ ID NO: 4); a CDR-H2 comprising the amino acid sequence ISGSGGTT (SEQ ID NO: 21); and a CDR-H3 comprising the amino acid sequence KGLWFGEGL (SEQ ID NO: 10).

32. The anti-VEGFR2 antibody or antigen binding fragment thereof according to any one of claims 1-31, wherein the antibody comprises an Fc sequence of a human IgG.

33. The antigen binding fragment of the anti-VEGFR2 antibody according to any one of claims 1-32, wherein the antigen binding fragment is selected from the group consisting of a Fab, Fab′, a F(ab)′ 2, a single-chain Fv (scFv), an Fv fragment, a diabody, and a linear antibody.

34. The anti-VEGFR2 antibody of any one of claims 1-33, wherein the antibody is a multi-specific antibody.

35. The anti-VEGFR2 antibody or antigen binding fragment thereof according to any one of claims 1-34 conjugated to a therapeutic agent.

36. The anti-VEGFR2 antibody or antigen binding fragment thereof according to any one of claims 1-34 conjugated to a label.

37. The anti-VEGFR2 antibody according to claim 36, wherein the label is selected from the group consisting of a radioisotope, a fluorescent dye, and an enzyme.

38. An isolated nucleic acid molecule that encodes the anti-VEGFR2 antibody or antigen binding fragment thereof according to any one of claims 1-34.

39. An expression vector encoding the nucleic acid molecule of claim 38.

40. A cell comprising the expression vector of claim 39.

41. A method of producing an anti-VEGFR2 comprising culturing the cell of claim 40 and recovering the anti-VEGFR2 from the cell culture.

42. A composition comprising the anti-VEGFR2 antibody or antigen binding fragment thereof according to any one of claims 1-35 and a pharmaceutically acceptable carrier.

43. A method of detecting a VEGFR2 protein in sample from a patient by contacting the anti-VEGFR2 antibody or antigen binding fragment thereof according to any one of claims 1-34 and 36-37 to the sample and detecting the anti-VEGFR2 antibody bound to the VEGFR2 protein.

44. The method according to claim 43, wherein the anti-VEGFR2 antibody or antigen binding fragment thereof is used an immunohistochemistry assay (IHC) or in an ELISA assay.

45. A method of treating pathological condition characterized by excessive angiogenesis in a subject, comprising administering an effective amount of the composition of claim 42 to the subject.

46. The method of claim 45, wherein the pathological condition characterized by excessive angiogenesis is selected from the group consisting of cancer, an ocular disease, or inflammation.

47. The method of claim 46, wherein the pathological condition characterized by excessive angiogenesis is cancer.

48. The method of claim 47, wherein the cancer is colon cancer, colorectal cancer, gastric cancer, gastroesophageal cancer, bladder cancer, lung cancer, or solid tumor.

49. The method of claim 48, wherein the cancer is lung cancer, and wherein the lung cancer is non-small cell lung cancer (NSCLC).

50. The method of claim 45-49, wherein the subject is further administered a therapeutic agent selected from the group consisting of an anti-neoplastic agent, a chemotherapeutic agent, a growth inhibitory agent and a cytotoxic agent.
